(19) United States
(12) Patent Application Publication
(10) Pub. No.: US 2005/0147984 A1
(43) Pub. Date: Jul. 7, 2005

(54) Interaction detection on several probe arrays

(75) Inventors: Thomas Ellinger, Jena (DE); Eugen Ermantraut, Jena (DE); Ralf Ehricht, Jena (DE); Joachim W. Engels, Kronberg (DE); Kerstin Jahn-Hofmann, Neu-Isenburg (DE); Nancy Holzhey, Jena (DE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Clondiag Chip Technologies GmbH, Jena (DE)

(21) Appl. No.: 10/789,081

(22) Filed: Feb. 27, 2004

Related U.S. Application Data
(63) Continuation of application No. PCT/EP02/09780, filed on Mar. 6, 2003.

(30) Foreign Application Priority Data
Aug. 31, 2001 (DE) 101 42 643.7

(51) Int. Cl. C12Q 1/68; C12M 1/34

(52) U.S. Cl. 435/6; 435/287.2

(57) ABSTRACT

The invention concerns a method for detecting interactions between probe molecules and target molecules, whereby marking target molecules is not required. The invention further concerns probe arrays and kits suited to such a method, as well as a method for production, quality control and standardization of probe arrays.

### CROSS-REFERENCE TO RELATED APPLICATIONS

This application is a continuation of PCT/EP02/09780, filed Sep. 2, 2002, which claims priority on the basis of German Patent Application No. 101 42 643.7, filed Aug. 31, 2001, the enclosures of said applications being incorporated herein.

### BACKGROUND OF THE INVENTION

The invention relates to a method for the detection of interactions between probe molecules and target molecules on probe arrays, whereby labelling of the target molecules can be omitted. Moreover, the invention relates to probe arrays and kits suitable for such a method and to a method for the production and quality monitoring of probe arrays.

Biomedical tests are frequently based on the determination of the interaction between a molecule which is present in a known quantity and at a known position (the molecular probe) and the molecule and/or molecules to be detected (the molecular target). Up-to-date tests are usually carried out with one sample in parallel on several probes (D. J. Lockhart, E. A. Winzeler; Genomics, gene expression and DNA arrays; Nature 2000, 405, 827-836). In this case, the probes are usually immobilised in a given way on a suitable matrix such as described in WO 00/12575, for example (compare e.g. U.S. Pat. No. 5,412,087, WO 98/36827) or manufactured synthetically (compare e.g. U.S. Pat. No. 5,143,854, U.S. Pat. No. 5,658,734, WO 90/03382).

The detection of such an interaction is usually carried out as follows:

The probe or probes are fixed in the known way on a certain matrix. The targets are brought into contact with the probes in a solution and incubated under defined conditions. On incubation, a specific interaction takes places between the probe and the target. The bond thus formed is substantially more stable than the bond of molecules for which the probe is unspecific. Subsequently, the system is washed with the corresponding solutions such that the molecules, which are not specifically bound, are removed.

For the detection of the interaction between the target and the probe, numerous methods are used nowadays, some of which are described as follows:

Fluorescence-based methods, in particular, are known as detection methods based on labelling of the target. For this purpose, the targets are provided with fluorescent labels before, after or in the course of the specific interaction with the probes. Because of the high local resolution and less effort being required in comparison with other conventional methods, analyses based on highly integrated probe arrays are thus selected, as a rule, by the fluorescent-optical method (A. Marshall, J. Hodgson, DNA Chips: An array of possibilities, Nature Biotechnology 1998, 16, 27-31; G. Ramsay, DNA Chips: State of the Art, Nature Biotechnology 1998, 16, 40-44).

Various methods are known for effectively labelling targets with fluorophors. On the one hand, conjugates of a fluorophor and/or an anchor group for fluorescent molecules and a reactive group are used in order to label targets by the chemical route. Various commercial products are available specifically to label nucleic acids, such as e.g. BiotinChemLink from Roche Biochemicals, Mannheim, Germany; Ulyssis from Molecular Probes, Eugene, Oregon, USA; Psoralen-Biotin from Ambion Inc., Austin, Tex., USA. Targets are formed as the products of the method which targets are internally modified with substituents. Since the latter have an influence on the hybridisation behaviour of the target in a manner difficult to predict, hybridisation conditions of optimum stringency, such as those required for the detection of point mutations, are more difficult to define.

Moreover, enzymatic activities are exploited in order to label target molecules with fluorophors. Nucleic acid targets are, for example, copied by means of polymerases, wherein fluorescent monomers and/or monomers coupled with anchor groups such as biotin, digoxigenin or similar substances are incorporated into the copy. In a further development of this cDNA protocol, it is possible to label two different samples, namely the sample to be analysed and a standard sample, with different dyes for quantitative array experiments and to hybridise them simultaneously with the array (comparative hybridisation), the signals generated by the sample to be analysed being compared with those of the standard sample and each array element thus undergoing an internal calibration. In this way, however, only one standardisation of two values which themselves have not been standardized in relation to each other is assured.

In the case of all labelling processes based on the copying of the target, it is a disadvantage that the individual target sequences are copied with different levels of efficiency and in extreme case, e.g. as a result of the formation of stable secondary structures, a loss of information can occur for certain targets. Moreover, internally labelled copies of the target with the disadvantages described above are formed also in this case in the same way as by the above-mentioned chemical methods.

This problem can be avoided by labelling outside the region to be detected, e.g. at the terminus of the molecule to be detected. For this purpose, template-independent polymerases such as the terminal deoxynucleotidyl transferase or poly(A)-polymerase are used which successively link bases with the 3′-terminus of DNA or RNA (G. Martin, W. Keller, Tailing and 3′-end labelling of RNA with yeast poly(A) polymerase and various nucleotides. RNA, 1998, 4, 226-230, V. Rosenmeyer, A. Laubrock, R. Seibl, Nonradioactive 3′-end labelling of RNA molecules of different length by Terminal Deoxynucleotidyl Transferase. Analytical Biochemistry 1995, 224, 446-449, D. Figeys, A. Renborg, N. J. Dovichi, Labelling of double stranded DNA by ROX-dideoxycytosine triphosphate using Terminal Deoxynucleotidyl Transferase and separation by capillary electrophoresis. Anal. Chem. 1994, 66, 4382-4383).

A general disadvantage of the enzymatic incorporation of bases modified with fluorophors or anchor molecules is that, as a rule, they are poor substrates for polymerases and are therefore incorporated inefficiently. This situation is particularly marked in the case of the above-mentioned template-independent polymerases so that the desired highly specific labelling can not be achieved in this way. In any case, efficient labelling can only be achieved with selected combinations of polymerase and labelled base.

Further restrictions result from the detection technique. The presently most sensitive fluorescence readers utilise the narrow spectral lines of laser sources to excite the fluorophors such that only those dyes can be used for a sensitive detection which can be excited by available lasers. A disadvantage of the fluorescence-based detection of probe arrays is the fact that, in comparison with radioactive labelling, the sensitivity is approximately 100 times lower (F. Bertucci et al., Sensitivity issues in DNA-array based expression measurements and performance of nylon micro-arrays for small samples. Human Molecular Genetics 1999, 8 (9), 1715-1722). Consequently, the detection of a target is frequently possible only after its amplification and/or after signal amplification. U.S. Pat. No. 4,683,202 discloses an amplification by PCR for qualitative detections. Quantitative assays on probe arrays require a process with linear amplification kinetics. Such a method has been described by J. Phillips, J. H. Eberwine (Antisense RNA amplification: A linear amplification method for analyzing the mRNA population from single living cell. Methods 1996, 10 (3), 283-288) and by Wang et al., (High fidelity mRNA amplification for gene profiling., Nat. Biotechnol. 2000, 18 (4), 457-459).

Apart from these methods based on fluorescence, methods for labelling the target are known which allow detection of the target on the basis of other effects: specifically for the use of wide range arrays, the target is frequently labelled radioactively. The interaction is detected by incubation with an X-ray film or a photo imager. Moreover, the target can be labelled with a dye and its presence detected by means of a photometer. In DE 19 543 232, labelling of the target with detection spherules is described, the presence of which is optically detected following the interaction of the target with the probes.

In DE 10 033 334, a method is disclosed in which the interaction of targets with specific probes on probe arrays is visualised by a reaction in the course of which an insoluble product is formed and deposited at the site of the interaction. The process implements signal amplification and is characterised by an extremely simple detector structure.

Nature Biotechnology 1998, 16, 725-727 describes the detection of the complex between target and probe by mass spectrometry. In addition, mass-sensitive methods such as surface plasmon resonance are used (J. M. Brockman et al., A multistep chemical modification procedure to create DNA Arrays on gold surfaces for the study of protein-DNA Interactions with surface plasmon resonance imaging, J. Am. Chem. Soc. 1999, 121, 8044-8051). The U.S. Pat. No. 5,605,662 discloses a method for the direct electrical detection of the interaction.

The detection methods based on labelling of the target have the common disadvantages that they are not standardisable and the introduction of labelling is complicated.

Moreover, methods for the detection of molecular interactions are known, wherein direct labelling of the target nucleic acid can be omitted.

In WO 92/01813, a method is disclosed according to which a plurality of copies of a circular single-stranded template can be produced by means of linear kinetics. In WO 2000/04193, the use of this mechanism referred to as RCA (Rolling Circle Amplification) for the detection of molecular interactions on probe arrays is described. Following a specific interaction between the probe immobilised at the 3′ end on the array and the target, an adapter oligonucleotide is hybridised with the target. This is composed of a sequence complementary to the target and a sequence which exhibits complementarity to a circular single-stranded DNA molecule. The two modules are linked to each other via a 5′-5′ bond. After adding the circular single-stranded DNA molecule, a polymerase and the corresponding partially labelled building blocks, the DNA synthesis according to the RCA mechanism takes place on the probes on which an interaction has taken place, in the process of which labelling and signal amplifycation is as a result of the incorporation of a plurality of labelled building blocks. This method is characterised by a high sensitivity and a high specificity due to the double specific hybridisation between the probe and the target and between the target and the adapter oligonucleotide. However, it is very complicated and unsuitable for detecting a plurality of different interactions on one probe array.

In the case of the technique referred to as sandwich-hybridisation, two probes are used which bind to different non-overlapping regions of the target nucleic acid (A. R. Dunn, J. A. Hassel, A novel method to map transcripts: evidence for homology between an adenovirus mRNA and discrete multiple regions of the viral genome. Cell 1977, 12, 23-36; M. Ranki et al., Sandwich hybridisation as a convenient method for the detection of nucleic acids in crude samples. Gene 1983, 21 (1-2), 77-85). One of the two probes acts as so-called capture probe, which allows specific binding of the target nucleic acid to a surface. The second target-specific probe has a detectable unit so that the target nucleic acid is labelled via hybridisation to this second probe. Such sandwich hybridisations consequently fulfil two functions, namely the increase in the specificity of detection by double interaction with two specific probes and the labelling of hybridised target nucleic acids by hybridising with the detection probe.

A survey of the modifications of the sandwich hybridisation method is provided by F. Lottspeich and H. Zorbas (editors.), in Bioanalytik, Spektrum, Akad. Verl., Heidelberg, Berlin, 1998. U.S. Pat. No. 4,486,539 describes the use of sandwich hybridisations for the detection of microbial nucleic acids. U.S. Pat. No. 5,288,609 discloses a sandwich method which allows the immobilisation, hybridisation and detection of the target nucleic acid in a single step. In U.S. Pat. No 5,354,657, a sandwich hybridisation method is described where the detection is based on the interaction of digoxin or digoxigenin with specific detectable antibodies. In U.S. Pat. No. 4,751,177, a method based on sandwich hybridisation is disclosed where a bispecific capture probe is used, one of whose specificities is utilised for hybridisation with the target DNA and its other specificity for hybridisation with a surface. In EP 0 198 662 and EP 0 192 168, methods based on sandwich hybridisation are described in the case of which the immobilisation of the resulting complex is preceded by hybridisation of the capture and detection probes with the target nucleic acid in solution.

U.S. Pat. No. 5,641,630 discloses a method in which a complex is initially formed between the target probe and the capture probe, which complex is subsequently immobilised and then detected by hybridisation with the detection probe. U.S. Pat. No. 5,695,926 describes a method where the capture probes have a length of 11 to 19 bases and are not immobilised covalently on a hydrophobic substrate. From U.S. Pat. No. 4,882,269, a process based on sandwich hybridisation is known which guarantees signal amplification.

The above-mentioned processes carried out by sandwich hybridisation have the common disadvantage that both a specific capture probe and a specific detection probe are required for the detection of each individual target. A similarly large number of specific detection probes is required for the parallel detection of a number of different targets, for example on probe arrays, whereby the above-mentioned processes can be used only up to a low degree of parallelisation.

In order to solve this problem, processes have been described in the state of the art, in which all targets of a target mixture are provided with a uniform adapter sequence by copying the target mixture by starting out from a primer which contains the adapter sequence at its 5′ end. The homogeneous and efficient introduction of the adapter sequence via an adapter primer, however, is disadvantageous in the -case of those targets which do not have the same sequence modules to which the primer can bind so that this process has only a limited range of applications.

A sensitive sandwich hybridisation method requiring the introduction of a uniform adapter sequence for the sensitive detection of interactions on probe arrays is based on the use of multiply labelled dendrimers (compare U.S. Pat. No. 5,487,973, U.S. Pat. No. 5,484,904, U.S. Pat. No. 5,175,270, commercially available from Genisphere Inc., Montvale, N.J., USA). In this process, copies of the target provided with an adapter sequence are incubated with a probe array, wherein a specific hybridisation takes place. The detection of the specific hybridisation is accomplished in a second step by hybridising a multiply labelled dendrimer which carries the complement of the adapter sequence, thus binding to the adapter sequence. As a result of the restrictions while introducing the adapter sequence via an adapter primer, the applicability of this process to targets with uniform sequence modules, such as polyadenylated eukaryotic RNA, is limited.

The above-described detection processes, in which direct labelling of the target is omitted, have the common disadvantage that they are parallelisable only to a limited extent, if at all, and are consequently not suitable for array-based detection methods. If the parallelisability of these methods is to be guaranteed, this would again involve a modification or copying of the targets and would consequently be complicated and not standardisable.

Up to now no methods have become known by means of which a plurality of different interactions between targets and probes on a probe array can be detected efficiently, i.e. in particular with a high sensitivity and specificity, homogeneously, i.e. with the same efficiency for different targets, and in parallel.

A strong need consequently exists for further methods for the detection of targets by means of probe arrays which do not exhibit the disadvantages of the state of the art.

A promising approach consists of dispensing with the labelling of the targets which is associated with the problems described above. Such methods are consequently based on labelling of the probes and the selective removal of probes after the interaction with the targets.

In WO 98/01533, such a probe array is disclosed in the case of which a plurality of cleavable signal elements is immobilised on the surface of a solid carrier. The signal elements comprise a chemical linker with a potential cleavage site or breaking site, in particular a siloxane group, a label being attached to the linker on a surface opposite to the site of immobilisation. The target specificity is provided by two oligonucleotide probes which are linked with the chemical linker below or above the siloxane cleavage site. By binding the target both to first and to the second oligonucleotide probe of a cleavable signal element, the label remains linked to the substrate surface in spite of a subsequent cleavage of the siloxane cleavage site. The presence or absence of signals following contact with the specimen and the contact with an agent cleaving the cleavage site, indicates the presence or absence of targets.

The synthesis of the signal elements described in WO 98/01533 is however extremely complicated so that efficient and homogeneous labelling of the probe array before the interaction with the target is not guaranteed. Moreover, in the method described in WO 98/01533 there is the possibility that the two probe sequences of a signal element do not interact jointly with a target molecule, as described above, but instead a target molecule hybridises in each case to each of the two probes. In the latter case, the labelling is detached from the substrate surface after the cleavage of the siloxane cleavage site and an erroneously negative measuring result is obtained.

In U.S. Pat. No. 4,876,187, probes for the detection of targets are described which have a label, a cleavable bond and an immobilisation anchor. The assay which is also described therein, making use of such probes comprises the steps of immobilisation, hybridisation, enzymatic cutting of the cleavable bond and detection of remaining labels coupled with the surface.

U.S. Pat. No. 4,876,187 does not describe a detection that can be carried out in parallel on a series of probes. In particular, the probes described therein are not suitable for array processes since an enzymatic cleavage of probes immobilised on a surface is extremely inefficient as a result of the spatial proximity of the cleavage site to the surface due to which a high background would be produced by erroneously positive signals. Moreover, the method of covalent immobilisation on the surface described in U.S. Pat. No. 4,876,187 is not specific for the reactive groups intended for this purpose such that the immobilisation also takes place on other groups within the probe. If the immobilisation site is located between the cleavage site and the label, the label remains on the surface also even after cleavage. Consequently, a quantitative removal of the labelling of probes, to which no targets have hybridised, from the surface, is not possible, which in turn produces a high background. A non-covalent immobilisation also described in U.S. Pat. No. 4,876,187 has proved to be non-stable so that probes are released into the solution even without cutting, leading to possibly erroneously negative measurement results. The method described in U.S. Pat. No. 4,876,187 consequently has no practical significance as a result of the above-mentioned problems.

The methods described in U.S. Pat. No. 4,775,619 and U.S. Pat. No. 5,118,605 circumvent the above-described high background problems by carrying out a so-called inverse assay. In this case, hybridisation of a target with an immobilised and labelled probe leads to the formation of a labile bond between the surface and the label such as a restrictase cleavage site or a chemically labile single stranded region. The cleavage of this labile bond leads to the detachment of the label which is then quantified in the solution. Such a procedure is not practicable for the array method, since a spatial resolution is no longer guaranteed if the detection does not take place via markers immobilised on the surface.

The transfer of the method described in U.S. Pat. No. 4,775,619 and U.S. Pat. No. 5,118,605 to array applications by recording the array before the interaction with the targets and after removing the labels, thus determining an interaction for those probes at which no signal is detectable after incubation with targets and cleavage of the labile bond, is also not possible, since the probe concentrations are usually considerably higher than the concentrations of the targets to be detected and consequently a strong signal is detected even also on array elements on which some of the labels have been cleaved as a result of the hybridisation of the probes with the targets. Specific detection, in particular of target molecules at low concentration, is not possible in this way.

U.S. Pat. No. 5,367,066, U.S. Pat. No. 5,552,538 and U.S. Pat. No. 5,578,717 disclose the structure and synthesis of oligonucleotides with chemically or physically cleavable bonds for use in hybridisation assays, for instance in the assays described in U.S. Pat. No. 4,775,619 and U.S. Pat. No. 5,118,605. The cleavability of the oligonucleotide probes is partly achieved by introducing bulky side groups which, however, negatively influence the hybridisation behaviour of the sequences. Moreover, the synthesis of the oligonucleotide building blocks described therein is complicated and, in particular, cannot be integrated directly into the standard DNA synthesis chemistry for the synthesis of the oligonucleotide probes. Finally, easy handling of the compounds is greatly restricted due to their partial sensitivity to light. As is well known, the efficiency of photochemical cleavages is not quantitative.

A further problem of the array experiments described in the state of the art consists of the fact that the degree of standardisation thereby achieved is unsatisfactory. For example, experiments carried out by different laboratories are, as a rule, not comparable with each other, the result being that their data cannot be collected and subjected to joint analysis. Even those experiments are comparable only to a limited extent which are carried out in one laboratory using different arrays of the same layout or samples which are different but have been worked up in the same way (compare e.g. Schuchhardt, J.; Beule, D.; Malik, A.; Wolski, E.; Eickhoff, H. L.; Herzel, H., Nucleic Acids Research, 2000, Vol. 28, No. 1, E47; Draghici, S.; Kuklin, A.; Hoff, B.; Shams, S. Drug Discovery & Development 2001, Vol. 4, No.3, 332; Zien A., Aigner T., Zimmer R., Lengauer T., Bioinformatics 2001 Jun;17:S323-S331; Tseng G. C., Oh M. K., Rohlin L., Liao J. C., Wong W. H. Nucleic Acids Res 2001 Jun 15;29(12):2549-2557, Lockhart D. J., Winzeler E. A., Nature 2000 Jun 15;405(6788):827-836).

One reason for the poor standardisability of the array-based detection methods of the state of the art is the variable quality of the arrays produced. In particular, it is normally not possible to determine the quality of the arrays prepared before the experiment is carried out and to take it into consideration during the subsequent evaluation. This necessarily leads to considerable errors.

A second source of errors for standardising measurement results obtained from array experiments arises if the detection method is based on the labelling of targets. Irrespective of the method used, target labelling is a method difficult to standardise which, simply because of the non-measurable variations in the quality of the starting material, takes place with greatly varying efficiency and is, additionally, influenced by the quality of enzymes, monomers, labelled building blocks, etc.

Approaches are described below which allow array experiments to be at least partly standardised. U.S. Pat. No. 5,800,992 describes a method in which two differently labelled samples are hybridised against one and the same array. A sample is used as reference. As a result, the method provides standardisation against variations in the spot quality. With respect to the different labelling efficiency, however, a standardisation is achieved only to a limited extent, since a not clearly defined sample, i.e. the sample to be examined, is merely standardised against another not clearly defined sample, namely the reference. A further disadvantage arises from the fact that the different labelling of both samples results in a different efficiency of incorporation of the dyes, a different quantum yield, quenching and a different dependence between the fluorescence and the concentration. These differences in the biochemical and physical properties represent further sources of error during standardisation.

A further approach is described by Selinger D. W., Cheung K. J., Mei R., Johansson E. M., Richmond C. S., Blattner F. R., Lockhart D. J., Church G. M., Nat. Biotechnol. 2000 Dec; 18(12):1262-8. In this case, an attempt is made to normalise both against variations in the spot quality and against variations in the quality of the labelling of the target. A normalisation against a varying labelling efficiency is achieved by adding externally produced targets of graduated known concentration, the so-called spiking targets, in a certain quantitative ratio to the sample. Additionally, oligonucleotides targeted against these spiking targets are arranged on the array, whose intensity, upon complete hybridisation, may be used for chip-to-chip standardisation. The disadvantage of this procedure is that certain restrictions regarding the sample quality which reduce the labelling efficiency, such as fragmentation of the nucleic acid or forming complexes with proteins, cannot be detected by means of the spiking probes.

Normalisation against variations in the spot quality is said to be achieved in Selinger et al., where spots occupied with a certain oligonucleotide, so-called calibration spots, are distributed over the entire array. A complementary oligonucleotide is added to the sample. The intensity of the calibration spots is measured and the deviation of each calibration spot from the average calibration spot intensity is used in order to determine a factor which is taken into consideration when calculating the results obtained with all other spots. Regarding the factor applicable to each spot, allowance is also made for the distance to the calibration spot. This procedure is extremely complicated and has the disadvantage that only global variations in the spot quality across the surface can be determined. Quality problems affecting only individual spots and which have no effect on the quality of the neighbouring spots cannot be determined with this method. Consequently, there is no process is available at present which both avoids the errors connected with the labelling of the target molecules and which allows the array quality at spot level to be included in the evaluation of array experiments.

At present two basically quite different processes are used for the production of probe arrays. In one method, separately synthesised probes, such as oligonucleotides, are attached to surfaces by means of robotic instruments, so-called spotters, which guarantee the site-specific deposition of minute quantities of liquid, and are covalently or non-covalently linked to the surface. The method operates serially. Each spot is occupied individually with the probe. The quality of the individual spots depends on a large number of factors which vary in the course of the serial production process such that the individual spots differ in their properties in a non-predictable manner.

Alternatively, DNA arrays are produced by the site-specific in situ synthesis of the probes, e.g. the oligonucleotide probes. The synthesis takes place in parallel, e.g. on a wave scale. The monitoring of the synthesis efficiency on each individual array element is difficult in the case of this highly parallel process which is frequently carried out on a wafer scale. Conventional monitoring approaches, such as trityl monitoring have proved to be unsuitable since they provide only a summary picture of the coupling efficiency across the entire wafer.

In the two production processes described above, a statement about the quality of the arrays produced can only be provided after they have been completed. For this purpose, for example chips taken at least randomly from a batch are hybridised with a standard sample and the chip quality is determined by means of the hybridisation signals. A disadvantage of this approach is that no reliable statements regarding the quality of each individual chip can be made where hybridisation is carried out on random samples. If sample hybridisation were carried out with each individual chip, their signals would have to be extinguished again after evaluation using a suitable method.

An alternative approach regarding the quality control of arrays produced by synthesis has been described by M. Beier and J. D. Hoheisel, Nucleic Acids Res. 2000, Vol. 28, No.4. According to this publication, a dye is coupled to the array surface via a labile linker following the synthesis of the oligonucleotides. The signal intensity determined on the individual spots is a measure of the synthesis yield. This method has the disadvantage that the labelling needs to be removed before the hybridisation assay and cannot be used simultaneously for the detection of hybridisation results.

### SUMMARY OF THE INVENTION

Accordingly, it is an object of the present invention is thus to provide a simple method for the detection of molecular targets by means of probe arrays, in which labelling of the targets can be dispensed with and which overcomes the disadvantages described above of the methods according to the state of the art.

Another object of the present invention is to provide probe arrays, in which, via cleavage of a labile bond, the label can be efficiently and selectively removed from those probes on which no specific interaction with targets has taken place, in particular also in cases where the probes have been immobilised on a surface.

It is, moreover, an object of the present invention to provide probe arrays where the labile bonds in the probes are such that the specificity of the interaction between the probes and the targets is only marginally influenced or not at all.

It is a further object of the present invention to provide probe arrays whose structure allows a standardised qualitative and, if necessary, quantitative evaluation of the array experiments, exceeding the possibilities known so far.

Another object of the present invention is to provide a method for the production of probe arrays which guarantees an efficient, homogeneous and/or parallel synthesis of the probes and, in particular, is as little influenced as possible by the incorporation of labile bonds into the probe molecules.

It is a further object of the present invention to provide a method by means of which the quality of each individual array can be examined immediately after its preparation without the need for further process steps such as sample hybridisations.

These and other objects of the present invention are achieved by providing the embodiments characterised in the patent claims.

A first aspect of the present invention is directed to a probe array for qualitative and/or quantitative detection of target molecules in a sample by molecular interactions between probe molecules and target molecules on the probe array, comprising an array surface and probe molecules immobilised on the array surface at defined sites, wherein the probe molecules have at least one label and at least one selectively cleavable bond between the site of their immobilisation on the array surface and the label.

A second aspect of the present invention is directed to a method for producing a probe array, comprising:
a) synthesizing probe molecules having a label and having a selectively cleavable bond between the site of their immobilisation on the array surface and the label; and
b) immobilizing the probe molecules via a defined position within the probe molecules at specific sites on the array surface.

A third aspect of the present invention is directed to a method for producing an array of probes on an array surface by in situ synthesis of the probe molecules on predetermined positions of the array surface, comprising:
a) providing an array surface which can be activated by suitable reagents or is provided with protective groups;
b) coupling or immobilising subunits of the probe molecules to be synthesised to predetermined sites on the array surface; and
c) synthesizing the probe molecules in situ following the coupling or immobilizing by incorporation of a label and a selectively cleavable bond between the site of the immobilisation of the probe molecules on the array surface and the label.

A fourth aspect of the present invention is directed to a method of controlling the quality of a probe array for qualitative and/or quantitative detection of target molecules in a sample by molecular interactions between probe molecules and target molecules on the probe array, comprising:
a) providing a probe array comprising an array surface and probe molecules immobilised on the array surface at defined sites, wherein the probe molecules have at least one label and at least one selectively cleavable bond between the site of their immobilisation on the array surface and the label; and
b) detecting the probe molecules in the form of signal intensities.

A fifth aspect of the present invention is directed to a method for qualitative and/or quantitative detection of target molecules from a sample to be analysed by molecular interactions between probe molecules and target molecules on probe arrays, comprising:
a) providing a probe array comprising an array surface and probe molecules immobilised on the array surface at defined sites, wherein the probe molecules have at least one label and at least one selectively cleavable bond between the site of their immobilisation on the array surface and the label;
b) incubating the probe array with the sample to be analysed;
c) selectively cleaving the selectively cleavable bond in the probe molecules; and
d) detecting the labelled probe molecule fragments which are retained on the array surface by an interaction with target molecules, in the form of signal intensities.

In some embodiments, the method is conducted by the following:
a) providing the probe array;
b) optionally, detecting the probe molecules synthesised or immobilised on the array surface in the form of signal intensities;
c) incubating the probe array with the sample to be analysed;
d) optionally, washing under conditions, under which a specific interaction between the target molecules and the probe molecules remains largely stable and unspecifically bound targets are removed;
e) optionally, detecting the probe molecules in the form of signal intensities;
f) selectively cleaving the selectively cleavable bond in the probe molecules;
g) optionally, washing in order to remove labelled probe molecule fragments which are not retained by an interaction with target molecules on the array surface;
h) detecting the labelled probe molecule fragments which are retained on the array surface by an interaction with target molecules, in the form of signal intensities; and
i) optionally, standardizing the signal intensities obtained in h).

A sixth aspect of the present invention is directed to a kit for qualitative and/or quantitative detection of target molecules from a sample by molecular interactions between probe molecules and target molecules on probe arrays, comprising:
a) a probe array comprising an array surface and probe molecules immobilised on the array surface at defined sites, wherein the probe molecules have at least one label and at least one selectively cleavable bond between the site of their immobilisation on the array surface and the label;
b) reagents for the selective cleavage of the selectively cleavable bond in the probe molecules;
c) hybridisation buffer; and
d) optionally, washing buffer.

A seventh aspect of the present invention is directed to a method for the production of monomer building blocks suitable for DNA synthesis, which can be used for the formation of a labile bond in probe molecules, comprising:
a) reacting a compound suitable as a protective group for the phosphotriester or phosphoramidite method to form a thiol;
b) esterifying the 5′-OH group of a nucleoside with an acid suitable as leaving group, to form an ester;
c) reacting the thiol of a) with the ester of b); and
d) activating the protected thiol at the 3′ position using the phosphotriester or phosphoramidite method.

A eighth aspect of the present invention is directed to a method for the production of monomer building blocks suitable for DNA synthesis, which can be used for the formation of a labile bond in probe molecules, comprising:
a) esterifying the 5′-OH group of a nucleoside with an acid suitable as leaving group, to produce an ester;
b) reacting the ester with a thioester;
c) saponifying the thioester to form a thiol;
d) protecting the thiol function with protective groups suitable for the phosphotriester or phosphoramidite method; and
e) activating the protected thiol at the 3′ position using the phosphotriester or phosphoramidite method.

A ninth aspect of the present invention is directed to the compound 5′-S-(dimethoxytrityl)-mercapto-5′-deoxynucleoside-3′-O-(2-cyanoethyl, N,N′-diisopropyl-phosphite.

### BRIEF DESCRIPTION OF THE DRAWINGS

FIG. 1 is an illustration of a scheme for the synthesis of a phosphoramidite building block.

FIG. 2 is a photograph of the gel representing the quantitative course of the cleavage of the modified oligonucleotide, wherein from left to right:
1. 5′-TTG ACG GTA TAT CT-3′ (14 mer control)+dye (XC+BP);
2. 5′-AGC CCT TAC T-3′ (10 mer control);
3. Model oligonucleotide 5′-AGC CCT TAC TTT GAC GGT ATA TCT-3;
4. Empty;
5. Model oligonucleotide (unmodified);
6. Cleavage reaction;
7. Cleavage reaction;
8. Model oligonucleotide (unmodified);
9. Cleavage reaction;
10. Cleavage reaction;
11. Model oligonucleotide (modified P—S bond);
12. Cleavage reaction;
13. Cleavage reaction;
14. Model oligonucleotide (modified P—S bond);
15. Cleavage reaction; and
16. Cleavage reaction.

FIG. 3a is a schematic arrangement of the probes in the experiment described in Example 5, and FIG. 3b is a photograph that illustrates detection of a hybridisation signal with the same intensity on both probe molecules as described in Example 5.

FIG. 4 is a schematic illustration of the principle of the experiment of Example 6.

FIG. 5 contains photographs of hybridisation signals following the cleavage of the phosphothioate with different concentrations of silver nitrate.

FIG. 6 contains photographs of the array following different steps of the experiment described in Example 7, wherein from left to right: after hybridisation of the target; after bond cleavage as well as after removal of the hybrids by melting; and again hybridisation under stringent conditions.

FIG. 7 is a schematic illustration of the method according to the invention.

FIG. 8 is a photograph of an array of the present invention following hybridisation (compare Example 9), wherein P identifies the tracks containing the unmodified oligonucleotide (match probe); the tracks characterised by PT contain the oligonucleotide of identical sequence (match probe) with the phosphothioate modification; and the tracks in between contain an oligonucleotide which differs from the two others by a T deletion approximately in the centre of the molecule (mismatch probe).

FIG. 9 is a photograph of an array of the present invention following cleavage of the phosphothioate bond and subsequent hybridisation (compare Example 10), whrerein P identifies the tracks containing the unmodified oligonucleotide (match probe); the tracks characterised by PT contain the oligonucleotide of identical sequence (match probe) with the phosphothioate modification; and the tracks in between contain an oligonucleotide which differs from the two others by a T deletion approximately in the centre of the molecule (mismatch probe).

FIG. 10 illustrates a scheme for the synthesis of 5′-O-(4,4′-dimethoxytrityl)-thymidylyl-(3′→5′)-3′-O-[(2-cyano-ethyl)-N-N-diisopropylamidophosphoramidite]-2′-deoxythymidine.

FIG. 11 illustrates a scheme for the synthesis of 5′-S-9-[4-methoxyphenyl)xanthene-9-yl]-mercapto-2′-deoxy-thymidine-3′-O-(2-cyanoethyl, N,N′-diisopropylphosphite).

FIG. 12 illustrates a scheme for the synthesis of amidites protected by 5′-S-dimethoxytrityl.

FIG. 13 illustrates scheme 1 for the synthesis of 1-{5′-O-(4,4′-dimethoxytrityl)-2′-O-{[(2-nitrobenzyl)-oxy]methyl}-β-L-ribofuranosyl}uracil 3′-[(2-cyanoethyl)-diisopropylphosphoramidite[ (1-5).

FIG. 14 illustrates scheme 2 for the synthesis of 1-{5 ′-O(4,4′-dimethoxytrityl)-2′-O-{[(2-nitrobenzyl)oxy]-methyl}-β-L-ribofuranosyl}uracil 3′-[(2-cyanoethyl)-diisopropylphosphoramidite] (2-6).

### DETAILED DESCRIPTION

The present invention consequently relates to probe arrays by means of which different interactions between probe molecules and target molecules on probe arrays can be detected highly specifically, highly sensitively and quantitatively.

The probe molecules which are arranged on the probe array according to the invention and are used to detect the molecular interactions with the target molecules comprise at least one label, i.e. a detectable unit, or an anchor-group which can be coupled to a detectable unit, and at least one predetermined breaking point i.e. a labile or selectively cleavable bond which can be specifically destabilised or cleaved, respectively.

The label with which the probes have been linked in the course of their production or in the course of the production of the probe array can preferably be detected in any phase of use of the array, i.e. also before the incubation of the array with the sample to be analysed, and consequently allows, among other things, an assessment of the quality of the array produced, in that the occupation density of each individual array element following the production of the array and before its use for detecting target molecules can be established. A further advantage of the probe array according to the invention consists of the fact that the signals used for quality control need not be eliminated before the actual array experiment but, rather, form the basis for the detection of specific interactions.

The selectively cleavable bond arranged between the label and the position of the linkage of the probes with the array surface makes it possible for the labelling and/or detectable unit used for quality control to be used also for the specific detection of the molecular interaction between probes and targets. In this connection, the predetermined cleavage site, or the selectively cleavable bond, is positioned within the probe molecule such that breaking of the bond leads to the detachment of the detectable unit, or the anchor group, with the detectable unit from the array surface. On the other hand, those labels remain linked to the array surface whose probe molecules have specifically interacted with target molecules, since the probe's cleavage product or the probe fragment linked to the label remains coupled to the second cleavage product of the probe which is immobilised on the surface of the array by interaction with the target.

The probe molecules of the probe array according to the invention consequently comprise a selectively cleavable bond of a nature such that its cleavage leads to detachment of the label from the probe molecules, at which no specific interactions with target molecules have occurred.

The structure of the probe array according to the invention allows assays for the detection of targets in a sample under analysis to be greatly simplified. The detection of the specific interaction between the probe and the target takes place via labels already attached during the production of the probe array. In this way, labelling of the target, which usually is a costly and labour-intensive method and, moreover, is frequently insufficiently efficient and homo-geneous, can be omitted.

Moreover, the probe array according to the invention guarantees that the multi-stage array-based detection methods which are dependent on the label of the target molecules and consequently the target molecules as such, can be converted into a homogeneous array-based assay which is completely independent of the target molecules. This provides a substantial enlargement of the field of application of array-based analyses.

By way of a standardisation of the signals by using internal, in particular non-cleavable control labels, the probe arrays according to the invention can, moreover be used also for quantitative assays.

The following terms and definitions are used within the scope of the present invention.

Within the scope of the present invention, probe or probe molecule means a molecule which is used for the detection of other molecules by specific characteristic binding behaviour, or certain reactivity. For the probes with a selectively cleavable bond, which are arranged on the array, any type of molecule can be used which are capable of being coupled to solid surfaces and exhibit a specific affinity. According to a preferred embodiment, these are biopolymers from the class of peptides, proteins, nucleic acids and/or their analogues. The cleavable probes are particularly preferably nucleic acids and/or nucleic acid analogues. Both DNA molecules and RNA molecules can be used as nucleic acids.

Within the scope of the present invention, target or target molecule means the molecule to be detected with a molecular probe. In a preferred embodiment of the present invention, the targets to be detected are nucleic acids. However, the probe array according to the invention can be used in an analogous way for the detection of the interactions between protein and probes, the interactions between antibodies and probes etc.

Within the scope of the present invention, the term “selectively cleavable bond” means a bond which differs from other bonds present in the probe molecule such that it can be cleaved specifically under certain conditions without the other bonds, in particular the bonds of the backbone of the probe molecules, being negatively affected. Preferably, the selectively cleavable bond is produced by the substitution of one or two atoms, if necessary, also three, four, five or several atoms in at least one monomer building block used for the construction of the probes. Preferably, the substitution takes place in the backbone of the polymer chain so that the recognition of the target is not affected by the functional groups of the probes, e.g. by nucleobases in the case of oligonucleotide probes. In the case of oligonucleotide probes, for example, preferably only one or two atoms of a nucleotide building block are thus preferably substituted, particularly preferably in the phosphate group of the nucleotide, e.g. O by S, thus providing a selectively cleavable bond. When nucleic acids are used as probes, for example, it is possible to cleave a selectively cleavable bond without cleaving the phosphodiester bonds of the probe. Within the scope of the present invention, the selectively cleavable bond is also referred to as labile bond or predetermined cleavage site.

Within the scope of the present invention, the term sample means a complex mixture containing a plurality of targets.

Within the scope of the present invention, labelling refers to a detectable unit, e.g. a fluorophor, or an anchor group to which the detectable unit can be coupled.

Within the scope of the present invention, probe array means an arrangement of molecular probes on a surface, the position of each probe being determined separately. In particular, within the scope of the present invention, probe array means a biochip and/or a microarray which has a high density of array elements and consequently allows simultaneous testing of a large number of probe-target interactions (so-called High Density Arrays or Microarrays). Preferably, arrays can comprise more than 100, particularly preferably more than 1,000 and more than 10,000 probe spots and even up to more than 100,000 probe spots per chip, or carrier. Such chips or carriers are commercially available and also described in the literature mentioned here (for a survey compare also Science (2002) 295, p. 60-172).

Within the scope of the present invention, the term “array element” should be understood to mean a certain area on a surface on which a uniform composition of probes is arranged, the sum of all occupied array elements being the probe array.

The probe array according to the invention used for the qualitative and/or quantitative detection of targets from a sample by molecular interactions between probe molecules and target molecules on the probe array comprises an array surface as well as probe molecules immobilised at defined sites on the array surface. Essential features of the probe array according to the invention are that the probe molecules have at least one label and, within the probe molecule, at least one selectively cleavable bond between the site of their immobilisation on the array surface and the label.

In a particularly preferred embodiment of the probe array according to the invention, the probes are oligonucleotides which have a selectively cleavable bond within their nucleotide sequence. For example, the oligonucleotide probes can be oligonucleotides of a length of from 10 to 100 bases, preferably 15 to 50 bases and particularly preferably 20 to 30 bases which are immobilised on the array surface.

The selectively cleavable bonds used within the scope of the present invention are characterised in that they influence the hybridisation behaviour of the probes, in particular their specificity and/or affinity for certain target molecules, not at all or only slightly. Moreover, the probe molecules of the probe array according to the invention preferably comprise labile bonds cleavable under conditions which do not negatively influence the interaction between the probe and the target and/or do not bring about any linkage between the labelled probe fragment and the array surface following the cleavage.

Moreover, the selectively cleavable bonds are preferably created in such a way that they are effectively cleavable even when the probes are immobilised on the array surface.

The selectively cleavable bond of the probe array according to the invention can preferably be selectively cleaved by chemical and/or physical methods.

An efficient cleavage at the surface is also guaranteed in particular by agents with a small size such as atoms and ions. The labile bond is therefore preferably selectively cleavable by simple chemical agents, e.g. by the addition of ions, particularly preferably of acid anions, base cations, fluoride ions and/or heavy metal ions such as mercury and/or silver ions.

In the production of the array by immobilisation of separately synthesised oligonucleotides, the selectively cleavable bond is stable under the conditions which are applied in the immobilisation of the probes on the array surface. If the production of the probes take place in situ by site-specific synthesis on the array surface, it is preferred that the labile bond can be produced efficiently in the course of the synthesis. The provision of the labile bond by phosphoramidite chemistry is particularly preferred. Incidentally, the same applies to the incorporation of the detectable unit.

Consequently, it is preferred that the selectively cleavable bond is present in a nucleic acid which can be produced by conventional DNA or RNA synthesis. Particularly preferably, the probe molecules of the probe array according to the invention comprise a nucleic acid of the formula A₁-S-A₂, S representing a nucleic acid and/or a nucleotide building block comprising at least one selectively cleavable bond, and A₁ and A₂ representing any nucleic acids or nucleic acid analogues. The probe molecule is immobilised on the surface of the probe array according to the invention via one of the two nucleic acids or nucleic acid analogues A₁ and A₂, whereas the other has at least one label. S is preferably a nucleotide dimer bridged by a selectively cleavable bond.

Examples of particularly preferred DNA nucleotide building blocks S comprising a selectively cleavable bond are indicated in the following formula I:

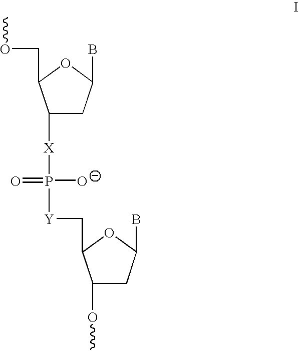

Here, X and Y can independently of each other be selected from the group preferably consisting of O, NH and S, wherein X and Y are not simultaneously O. B represents a nucleobase such as the purine derivatives adenine and guanine and the pyrimidines cytosine and thymine.

The selectively cleavable bond within the nucleotide sequence of such oligonucleotide probes is preferably a phosphothioate bond or a phosphoramidite bond. Particularly preferably, the phosphothioate bond, i.e. a sugar-O—P—S-sugar bond replaces a phosphodiester bond, i.e. a sugar-O—P—O-sugar bond of an unmodified oligonucleotide. In this embodiment, two nucleosides are bound by a phosphothioate bond.

Alternatively, the selectively cleavable bond within the nucleotide sequence can also be another sulfur or nitrogen modified ester bond, such as a phosphonothioate bond.

Further examples for the provision of selectively cleavable bonds in the probe molecules of the probe array according to the invention are amide groups, 1,2-diol groups, disulfide groups and/or sulfonyl groups as well as other groups described in U.S. Pat. No. 5,118,605 which are cleavable under the conditions detailed therein. However, these groups are less preferred since their incorporation, among other things, into oligonucleotide probes is not possible by conventional nucleic acid synthesis.

Alternatively, physical methods can also be used for the cleavage of the selectively cleavable bond in the probe molecules. In this way, the selectively cleavable bond can be selectively cleaved for example by photolysis. Nucleotide building blocks, which comprise a photolytically selectively cleavable bond and can be used for the synthesis of the probe molecules of the probe array according to the invention, are described for example in U.S. Pat. No. 5,367,066, U.S. Pat. No. 5,552,538 and U.S. Pat. No. 5,578,717.

Other examples of particularly preferred RNA nucleotide building blocks comprising a bond which is selectively cleavable by chemical or physical means, are indicated in the following formula II:

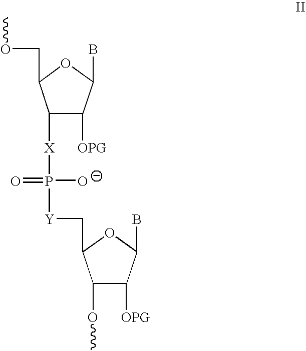

wherein X and Y can independently of each other be selected from the group preferably consisting of O, NH and S, wherein X and Y are not simultaneously O if PG is not a labile protective group.

Preferably, PG is selected from the group consisting of H and labile protective groups such as 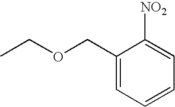
(photolabile) or 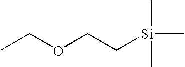
(labile e.g. against fluorine ions).

B in formula II represents a nucleobase such as the purine derivatives adenine and guanine and the pyrimidines cytosine and uracil.

A further subject of the present invention consequently is the use of RNA nucleotide building blocks with selectively cleavable bonds, in particular of photolabile RNA nucleotide building blocks as building blocks of DNA oligonucleotide probes. By means of such photolabile RNA nucleotide building blocks, a photolytically selectively cleavable group is provided. Preferably, this photolytic cleavage takes place in an alkaline medium, particularly preferably at a pH greater than 10.

However, probe molecules with selectively cleavable bonds are preferred which are stable under normal atmospheric, temperature and light conditions.

In an alternative embodiment, the labile bond is selectively cleavable by enzymatic methods. Examples of nucleotide building blocks comprising such labile bonds are described in U.S. Pat. No. 4,775,619 and U.S. Pat. No. 4,876,187. However, within the scope of the present invention, enzymatic methods for cleaving the selectively cleavable bonds are less preferred since enzymatic activities are greatly hindered by the proximity of the selectively cleavable bond to the surface as a result of the immobilisation of the probe molecules. Consequently, an enzymatic cleavage reaction has only a very low level of efficiency resulting in an undesirably high signal background as a result of erroneously positive measurement results. In a preferred embodiment of the probe array according to the invention, the selectively cleavable bond cannot therefore be selectively cleaved by enzymatic methods.

In a further preferred embodiment of the probe array according to the invention, the selectively cleavable bond is situated approximately in the centre between the site of immobilisation of the probe on the array surface and the position of the labelling of the probe. In this way, it is guaranteed that the likelihood of an interaction of the target with the immobilised probe fragment corresponding to the residue of the probe remaining on the surface after cleavage of the bond is greatly reduced and/or almost excluded. On the other hand, if the selectively cleavable bond is located too near to the array surface, the complex of probe molecule and target molecule is no longer sufficiently stabilised after cleavage, since the hybridisation of the target with the probe fragment immobilised on the array surface is not sufficiently stable. This would lead to erroneously negative measurement results.

In a preferred embodiment of the probe array according to the invention, the selectively cleavable bond can be cleaved quantitatively. This guarantees that the labels are completely removed from those probes at which no interaction with the targets to be detected has taken place. On the other hand, those labels remain linked to the array surface whose probes have undergone specific interaction with targets since the probe's cleavage product linked to the label remains coupled, via the interaction with the target, with the probe's second cleavage product still being immobilised on the surface of the array.

It is also preferred that the selectively cleavable bond in the probes is such that it does not interfere with the molecular interaction between the probes and the targets. In this way, it is guaranteed that the specificity of the detection is not diminished by the modification of probe molecules on the introduction of a selectively cleavable bond.

Moreover, it is preferred for the selectively cleavable bond to be such that the interaction between the probes and targets is fully retained during the selective cleavage of the selectively cleavable bond.

In one embodiment of the probe array according to the invention, all the probes on the probe array have both at least one cleavable bond as well as at least one label. In further embodiments, it may be preferred for at least one probe to possess both at least one cleavable bond and at least one label whereas other probes have only at least one label or only at least one cleavable bond or neither a label nor a cleavable bond.

Preferably, probe molecules not having a selectively cleavable bond are arranged on at least one array element of the probe array. In this preferred embodiment, all probes of the probe array have a label. Some of the probes additionally possess a labile bond whereas probes used for standardisation purposes have no cleavable bond. The latter will not lose their label in the course of the assay as a result of the specific cleavage of the labile bond. However, since they are exposed to all the steps of the assay, they, like all other probes, are subject to the unspecific decrease in the labelling signal connected with these steps. Consequently, those probes can be used as standards whose signals provide the upper limit of the signals to be detected after the assay and consequently the upper limit of the dynamic range of the analysis. These probes can consequently be used for the mutual standardisation of different experiments.

In a particularly preferred embodiment, the probe molecules, which are not provided with a selectively cleavable bond, are arranged on different array elements which differ in their degree of labelling. Thus, some array elements can contain only labelled probes whereas in other array elements a mixture of labelled and non-labelled probes is arranged. Particularly preferably, array elements provided with a mixture of labelled and non-labelled probes are present in the form of a dilution series. This means that the array elements concerned differ from each other in a characteristic and defined manner by the ratio of labelled to unlabelled probes.

In the case of arrays according to the invention produced by the immobilisation of separately synthesised probes, the different degree of labelling can be achieved by mixing of labelled and unlabelled probes before immobilisation. In the case of arrays produced by in situ synthesis, the monomer effecting labelling, for example, can be mixed in the course of coupling with a unlabelled monomer of the same reactivity.

The array elements exhibiting a stepped degree of labelling can be used for the standardisation of the experiment. On the one hand, they represent the upper and the lower limit of the detectable range. On the other hand, the signal intensities determined on the array elements can be plotted against the degree of labelling of the probes in the course of the evaluation such that a characteristic curve of the array experiment is obtained against which the experimental values, i.e. the values obtained on array elements with probes having a labile bond, can be balanced.

Those probes not provided with a labile bond can be selected from among a large number of chemical bonds suitable for coupling to surfaces. Preferably, however, probes not provided with a labile bond are molecules which, chemically to a large extent resemble those probes used for the detection of interactions such as e.g. oligonucleotide probes in the case of assays based on hybridisation. The probe molecules are particularly preferably oligonucleotides of a defined or randomised sequence.

In a further embodiment, the array elements used for standardisation can also be provided directly, i.e. without linkage to a probe molecule, with detectable units. In this embodiment, detectable units without linkage to a probe molecule are arranged on at least one array element. Different signal intensities are then achieved preferably by mixing the detectable units with non-detectable units of the same reactivity when these are coupled to the surface.

In a further embodiment of the probe array according to the invention, all probes with labile bonds have a specific affinity to the target to be expected in the sample to be analysed.

Preferably, however, probe molecules are arranged on at least one array element of the probe array according to the invention which probe molecules have no or at least no specific affinity to the target molecules. In other words, on some array elements, probes are deposited which have no affinity to targets to be expected in the sample and/or whose affinity to targets in the sample does not lead to a detectable signal. Following the cleavage of the labile bond, the detectable unit in these probes is not held back by a specific interaction between the probe and the target on the array. Consequently, these probes serve as controls for the efficiency of the cleavage reaction. They define the signal background inherent in the experiment and thus the lower end of the detectable range.

Preferably, the probe molecules having no or at least no specific affinity to the target molecules are oligonucleotides with a defined or randomised sequence. It is thus possible in the case of nucleic acids and/or nucleic acid analogues used as probes to select the sequence of the probes such that they have no significant complementarity to targets in the sample. This is practicable in particular for samples with a qualitatively defined composition. In the case of samples with an unknown composition, at least partly randomised sequences can be used instead of defined sequences. Although these may comprise sequences which are complementary to the targets present in the sample, the proportion of each individual specific sequence thus produced is only ¼ⁿ, in the case of completely randomised array elements, n representing the length of the probe. As a result of the small quantity of each individual specific sequence, their interaction with targets present in the sample leads to a summary signal which is below the detection limit.

In an embodiment of the probe array according to the invention, all probes which are provided with a cleavable bond and directed to target sequences are complementary to sequences occurring naturally in the target.

Alternatively, probe molecules are arranged on at least one array element, which probe molecules have a specific affinity for target molecules added externally to the sample preferably in a known concentration. In this embodiment, probes with labile bonds are arranged on the array which are directed to targets added to the sample for purposes of standardisation. This is referred to as “spiking”. A concentration gradation of different targets added as spiking samples allows a further standardisation of the assay.

In a further preferred embodiment of the probe array according to the invention, so-called control probes are arranged on at least one array element, which control probes have a label and a selectively cleavable bond arranged between the label and the immobilisation site of the probe on the surface. Such control probes have a specific affinity, i.e. in the case of oligonucleotide probes a complementarity either to an externally added target or to a target present in sufficient concentration in all samples to be analysed with the array. Sufficient concentration in this context means a concentration of target molecules which leads to a significant, i.e. clearly detectable signal, following the interaction with the probes.

Preferably, the array elements on which such control probes are arranged are distributed over the entire surface of the array; particularly preferably, they are distributed evenly. A distribution over the entire surface of the array in the context of the present invention means that, starting from the centre of the array surface, array elements with such control probes are present at different distances and in different directions. An even distribution preferably means an arrangement of the array elements with such control probes as a uniform grid, e.g. 10×10 grid, in which every tenth array element is such an array element with control probes. This embodiment allows the normalisation of experimental variations which may occur after the production of the array, among other reasons depending on the site of the array element on the array surface.

As mentioned above, the label coupled to the probes, preferably the oligonucleotide probes, is preferably a detectable unit or a detectable unit coupled to the probes via an anchor group. As regards the possibilities of detection and/or labelling, the probe array according to the invention has proved to be extremely flexible, in comparison with the probe arrays of the state of the art, since the labelling efficiency does not depend on the sequence, the structure or other characteristics of the (unknown) target molecules. Processes using the probe array according to the invention are thus compatible with a large number of physical, chemical or biochemical detection methods. The only requirement is that the unit or the structure to be detected can be coupled directly to a probe, e.g. an oligonucleotide, or linked to it via an anchor group that can be coupled with the oligonucleotide.

The detection of the label can, for example, be based on fluorescence, magnetism, charge, mass, affinity, enzymatic activity, reactivity, gold labelling and the like. Preferably, the label can be detected in case of the probe arrays according to the invention in any application phase of the array. In this way, an assessment of the quality of the array, for example, is guaranteed in that the occupation density of each individual array element can be determined.

The probe array according to the invention also allows a detection by means of methods not based on fluorescence. However, the label is preferably based on the use of fluorophor-labelled structures or building blocks. In connection with fluorescence detection, the label may be any dye that is linkable to probes during or after synthesis. Examples of these are Cy dyes (Amersham Pharmacia Biotech, Uppsala, Sweden), Alexa dyes, Texas Red, fluorescein, rhodamine (Molecular Probes, Eugene, Oreg., USA), lanthanides such as samarium, ytterbium and europium (EG&G Wallac, Freiburg, Germany).

Within the scope of the present invention besides fluorescent markers, also luminescent markers, metal markers, enzyme markers, radioactive markers and/or polymer markers can be used as labelling and/or detection unit coupled to the probes.

Similarly, a nucleic acid can be used as label (tag) which can be detected by hybridisation with a labelled reporter probe (sandwich hybridisation) . Various molecular-biological detection reactions, such as primer extension, ligation and RCA, are used for detecting the tag.

In an alternative embodiment of the probe array according to the invention, the detectable unit is coupled to the probes via an anchor group. Anchor groups preferably used are biotin, digoxigenin and the like. In a subsequent reaction, the anchor groups are reacted with specifically binding components, such as streptavidin conjugates or antibody conjugates which are detectable themselves or trigger a detectable reaction. When using anchor groups, the conversion of the anchor group into detectable units can take place before, during or after the addition of the sample comprising the targets as well as before, during or after the cleavage of the selectively cleavable bond in the probes. Preferably, however, the conversion of the anchor groups into detectable units takes place after the production of the probe array and, in particular, before cleavage of the probes in order to verify the quality of the probe array produced.

According to the invention, labelling can also be effected by interaction of a labelled molecule with the probe molecules. Labelling can take place for example by the hybridisation of an oligonucleotide labelled as described above, with an oligonucleotide probe.

Other labelling methods and detection systems which are suitable in the context of the present invention are described for example by Lottspeich and Zorbas, Bioanalytik, Spektrum Akademischer Verlag, Heidelberg, Berlin 1998, Chapters 23.3 and 23.4.

In a further alternative embodiment of the probe array according to invention, detection methods are used which lead to the provision of an adduct with a certain solubility product resulting in precipitation. Substrates are used for labelling which can be converted into a poorly soluble, usually stained product. It is, for example, possible to use enzymes in this labelling reaction which catalyse the conversion of a substrate into a poorly soluble product. A number of reactions suitable for providing a precipitate on array elements and possibilities for detecting the precipitate which can be considered are described in International Patent Application WO 01/07575, for example.

A further essential aspect of the present invention is a method for the preparation of a probe array according to the invention as described above, which comprises the following steps:
a) Synthesis of probe molecules having a label as well as a selectively cleavable bond between the site of their immobilisation on the array surface and the label; and
b) site-specific immobilisation of the probe molecules via a defined position within the probe molecule on the array surface.

In an alternative embodiment of the method for producing a probe array according to the invention as described above, production takes place by the in situ synthesis of the probe molecules at predetermined positions of the array surface, comprising the following steps:
a) Providing an array surface which can be activated with suitable reagents or is provided with protective groups;
b) Coupling or immobilising subunits of the probe molecules to be synthesised at predetermined sites on the array surface, preferably by the deposition of subunits, coupling taking place at predetermined sites, preferably by the activation of, or the removal of protection (deprotection) from, the array surface and subsequent coupling of the sub-unit;
c) in situ synthesis of the probe molecules based on the subunits coupled or immobilised in step b) with the incorporation of a label and a selectively cleavable bond between the site of immobilisation of the probe molecules on the array surface and the label.

Suitable reagents for activating the array surface in step b) of the above-mentioned method or suitable protective groups for the array surface are know to the person skilled in the art.

The immobilisation of molecules on the array surface can take place either specifically or unspecifically. The specific immobilisation presupposes selectivity of the interaction of certain chemical functions of the molecule to be immobilised and the surface of the substrate. An example of a specifically non-covalent immobilisation is the binding of biotin-labelled nucleic acid to a substrate coated with streptavidin. Amino-modified nucleic acids can be immobilised specifically via the reaction of the amino group with an epoxide, a carboxy function or an aldehyde. In the method according to the invention, the immobilisation is preferably carried out via a terminal phosphate group of the probe or a monomer building block of a biopolymer probe on an aminated surface.

The unspecific immobilisation takes place by means of a large number of mechanisms and chemical functions and can be both covalent and non-covalent. An example in this respect is provided by the immobilisation of nucleic acids on poly-L-lysine but also the immobilisation of chemically non-modified nucleic acids on epoxidised, aminated substrate surfaces or those occupied by aldehyde functions.

Preferably, the immobilisation of the probes on the array surface takes place covalently.

Numerous processes exist for depositing small quantities of material on predetermined sites on a substrate in connection with the preparation process according to the invention; some of them will be detailed in the following. A number of such processes have been described in D. J. Lockhart, E. A. Winzeler; Genomics, gene expression and DNA arrays; Nature, Vol. 405, p. 827-836, June 2000, for example. In U.S. Pat. No. 6,040,193, a process is described which allows arrays to be set up by depositing droplets from a capillary, making use of hydrophilically structured areas. In EP 0 268 237, a jet head is described which equally guarantees such as deposition of small quantities of material. The deposition of droplets by means of transfer needles is well known. The needles may also comprise a slit such that liquid can be deposited at a large number of sites on a substrate in analogy to the nib of a fountain pen (compare e.g. U.S. Pat. No. 6,269,846, U.S. Pat. No. 6,101,946, U.S. Pat. No. 6,235,473, U.S. Pat. No. 5,910,288). In U.S. Pat. No. 4,877,745, a device is described which permits the production of molecular arrays by pipetting small quantities of material. In U.S. Pat. No. 5,731,152, a pipetting tool is described which simultaneously allows the deposition of a large number of different probes. In U.S. Pat. No. 5,551,487, a jet head borrowed from ink jet technology for producing molecular probe arrays is described. Special tools for the spot-type deposition of material on surfaces have also been described and mentioned, inter alia in U.S. Pat. No. 5,807,522. The possibility of immobilising probes using electrical fields is also known from U.S. Pat. No. 5,434,049 and WO 97/12030 A1.

Due to the fact that, during the production of the probe array, the probes can be immobilised via a defined position within the probe molecule on the array, upon the cleavage of the labile bond the labels of those probes, at which no specific interaction with the target has taken place, can be removed efficiently from the surface.

In particular, the method according to the invention avoids further stable contacts of the probes and surface from being formed in the area between the labile bond and the detectable unit.

Such a specific linkage between the probe and the surface can be ensured only to a limited extent by immobilising probes that are synthesised separately and completely, i.e. before immobilisation. Consequently, such covalent immobilisation processes are not preferred for the preparation process according to the invention. This applies also to non-covalent immobilisation methods which do not guarantee a sufficient stability of the linkage for certain applications.

On the other hand in the context of the present invention it has been found that a highly defined linkage of the probes to the surface can be achieved by in situ synthesis of the probes on the surface. Synthesis of the probes, in particular when oligonucleotide probes are used, thus preferably takes place in situ on defined positions of the array surface.

The in situ synthesis of probe molecules on surfaces is a special case of immobilisation. In this case, monomers of a polymeric compound are immobilised on the surface, the immobilisation being preferably covalent. Subsequently, the probe molecule is produced synthetically in situ on the surface. Particularly preferably, the synthesis of oligonucleotide probes takes place in situ, i.e. at the solid phase using phosphoramidite building blocks.

In the following, a number of processes will be described which are suitable for the preparation of molecular probe arrays in connection with the present inventions, especially using the phosphoramidite method for nucleic acid synthesis. The processes are detailed by way of examples without any claim to completeness, further processes and process extensions being known and conceivable.

In U.S. Pat. No. 5,658,734, a process is described by which photoresists are used for the determination of the probe elements. In U.S. Pat. No. 6,001,311, U.S. Pat. No. 5,985,551 and U.S. Pat. No. 0,574,796, processes are described which allow the successive arrangement of oligonucleotides and other polymers using specially structured substrates having hydrophilic reactive areas and hydrophobic non-reactive areas and by employing pipettes. From U.S. Pat. No. 5,885,837, U.S. Pat. No. 5,384,261, WO 93/09668, WO 97/33737 and WO 98/36827, it is known that mechanical barriers may be suitable to allow the synthesis of polymers at certain sites on a surface. Spotting of monomers or reagents is described in DE 197 06 570. From WO 90/15070, EP 0 386 229, U.S. Pat. No. 5,436,327, U.S. Pat. No. 5,667,667, WO 98/56505 and WO 95/21265, synthetically constructed probe arrays are also sufficiently well known. In U.S. Pat. No. 6,239,273, printing technology methods have been adapted for the preparation of probe arrays.

In the production process according to the invention, labile bonds whose synthesis is incorporated as smoothly as possible into the in situ synthesis of the arrays taking place according to the phosphoramidite method are preferred. Particularly preferably, the selectively cleavable bond is a modified phosphodiester bond bridging two nucleosides, e.g. it is a phosphothioate bond bridging two nucleosides.

A defined degree of labelling on an array element required for certain applications of the probe array according to the invention can, for example, be achieved by adding a mixture of a labelled monomer, preferably an nucleotide monomer, and an unlabelled monomer, preferably an nucleotide monomer of the same reactivity, preferably in a defined ratio, during the synthesis.

It is a further object of this invention to provide suitable monomer building blocks for the probe synthesis, especially according to the phosphoramidite method for the synthesis of the labile bond.

According to the invention, this object is achieved by providing a method for the production of monomer building blocks suitable for nucleic acid synthesis, which can be used for the formation of a labile bond in probe molecules, the method comprising the following steps:
a) Esterification of the 5′-OH group of a nucleoside with an acid suitable as leaving group;
b) Reaction of the ester with a thioester;
c) Saponification of the thioester to form a thiol;
d) Protection of the thiol function with protective groups suitable for the phosphotriester or phosphoramidite method;
e) Activation of the protected thiol at the 3′ position using the phosphotriester or phosphoramidite method.

In a further method according to the invention for the production of monomer building blocks suitable for the nucleic acid synthesis, sulfur is introduced into the nucleoside via a protective group suitable for the phosphotriester or phosphoramidite method. Such a method according to the invention preferably comprises the following steps:
a) Reaction of a compound suitable as a protective group for the phosphotriester or phosphoramidite method, preferably an alcohol such as triphenyl methanol or dimethoxytriphenyl methanol, to form a thiol;
b) Esterification of the 5′-OH group of a nucleoside with an acid suitable as leaving group;
c) Reaction of the thiol from step a) with the ester from step b);
d) Activation of the protected thiol at the 3′ position using the phosphotriester or phosphoramidite method

Suitable reagents and reaction conditions are known to the person skilled in the art, particularly in consideration of the syntheses described in Examples 1 and 14. Especially DMTr-(dimethoxytrityl), MMTr-(monomethoxytrityl), Tr-(trityl), 9-phenylxanthene-9-yl-, pixyl groups and silyl groups are suitable as protective groups. Suitable leaving groups are in particular tosylate, mesylate and chloride.

The compound trityl-5′-S-thymidine has been described by M. Mag, S. Lücking and J. W. Engels in Nucleic Acids Research (1991) Vol.19, No. 7 1437-1441. When these building blocks are used in the oligonucleotide synthesis according to the phosphodiester method, the trityl group is usually cleaved with an aqueous silver nitrate solution during the synthesis cycle in order to be able to continue the synthesis. When an aqueous cleavage solution is used, splitting off is usually not carried out on the synthesiser device since the oligonucleotide synthesis must be carried out under anhydrous conditions. During cleavage on the synthesiser, cleavage of the trityl group would have to be followed by a specific wash programme. The use of a DTT solution containing water as a solvent as well to reduce S—S bonds formed also reduces the coupling yielded in the subsequent synthesis sequence. Instead of a trityl protective group of the modified 5′-thio-amidite, a 4,4′-dimethoxytrityl protective group is thus preferably used whose cleavage can be effected with TCA or DCA in the standard cycle.

Further subject matters of the present invention consist of the monomer building blocks produced by the above synthesis according to the invention, such as 5′-S-(4,4′-dimethoxytrityl)-mercapto-5′-deoxynucleoside-3′-O-(2-cyanoethyl, N,N′-diisopropyl phosphite).

A further monomer building block according to the invention is 5′-S-9-[4-methoxyphenyl)xanthene-9-yl]mercapto-2′-deoxynucleoside-3′-O-(2-cyanoethyl, N,N′-diisopropyl phosphite), which can be produced in high yields.

Alternatively a corresponding dimer can be provided as building block for the phosphoramidite synthesis. Since, when using a dimer, cleavage of the 4,4′-dimethoxytrityl group directly during sulfur modification becomes superfluous becomes no longer necessary and thus also no disulfide bonds can be formed, an improvement in the yield and the use of a standard cycle with an extended coupling time are possible when using a dimer. The synthesis of an exemplary dimer is described in Example 12.

Further subject matters of the present invention consequently are nucleotide dimers with the following formula III which, among other things, can be produced from the above-mentioned monomer building blocks according to the phosphoramidite method, for example.

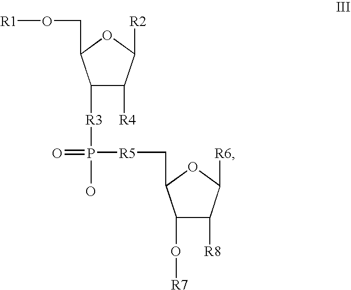

wherein R1 is preferably selected from the group consisting of DMTr-(dimethoxytrityl), MMTr-(monomethoxytrityl), Tr-(trityl), 9-phenyl xanthene-9-yl, pixyl and silyl groups; R2 and R6 independently from each other preferably represent A, G, C, T and U; R3 is preferably selected from the group consisting of O, S and NH; R4 is preferably selected from the group consisting of H, OH and O-alkyl; R5 is preferably selected from the group consisting of O, S and NH; R8 is preferably selected from the group consisting of H, OH and O-alkyl; and R7 is preferably selected from one of the two following groups with the formulae IV and V:

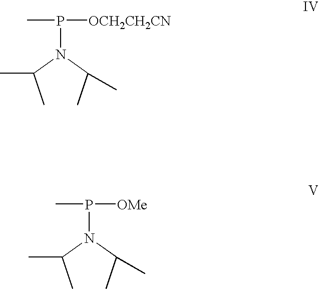

Obviously, the dimers according to the invention with the formula III can also be used as nucleotide dimers S in the production process according to the invention for the construction of probe molecules comprising a nucleic acid of the above-mentioned formula A₁-S-A₂.

The construction of the probe arrays according to the invention, or the arrays produced by the method according to the invention permit a reliable, easy to monitor degree of standardisation not hitherto attained and thus a high standard of quality. Since the probes on the probe array are provided with a label during the preparation of the array, the quality of the array produced can be verified by means of a simple imaging process. The intensity of the particular spots is a measure of the synthesis and immobilisation efficiency of the probe concerned.

A further advantage, besides the possibility of carrying out quality control of the array produced, consists of the possibility of standardisation and consequently of verification of the measured results obtained by using the probe array according to the invention. It is thus possible, for example, to carry out random functional tests, so-called standard assays. In this case, for example, a mixture of targets defined with respect to its concentration and composition may interact with the probe array or chip and the detection reaction may be carried out subsequently. The result of the interaction of the probes with the targets following the cleavage of the probes, which have not selectively interacted with the targets, is documented by means of an imaging process. The comparison of the experiments before and after the addition of the targets permits statements to be made regarding the quality of immobilisation or synthesis of the probes concerned.

Moreover, as a result of the special structure of the probe array according to the invention, a standardised evaluation of the assay is made possible. This is based on the fact that visual records of different points in time of the experiment are obtained using the probe array according to the invention, i.e. those obtained before the addition of the sample and those obtained after the interaction of the probes with the targets. By appropriate mathematical procedures it is possible, by including these data and the data obtained during the quality control of the array, to standardise and verify experimental results to a degree hitherto not attained for array applications. The probe arrays according to the invention thus allow a new quality level to be achieved in array experiments and open up new fields of application for array technology.

The method for the quality test on the probe arrays produced and the standardisation and normalisation of the detection of probe/target interactions when using the probe arrays according to the invention are also a subject of the present invention and will be described in detail below.

A further aspect of the present invention consequently relates to a method for testing the quality of the above-described probe arrays, or the probe arrays produced according to the above-described method, comprising the following steps:
a) Providing a probe array according to the invention;
b) Detecting the synthesised probe molecules immobilised on the array surface.

The advantage of the method according to the invention for quality control consists of the possibility of determining the quality of each array after its production without the need for further process steps. This is guaranteed in that the probes on the probe array are provided with a detectable unit after completion of the probe array.

In connection with the quality management, a directly detectable labelling such as e.g. the use of fluorescent labels and/or radioactive labels is preferred. In alternative embodiments, however, anchor groups or units detectable by secondary reactions can also be used.

Usually, the occupation density of the individual array elements is determined for each array after its completion based on the intensity of the signals generated by the labels. This is preferably carried out by means of an imaging method, which depicts the signal intensity in the form of degrees of greyness, which can then be evaluated quantitatively by suitable software tools such as e.g. Iconoclust (Clondiag, Jena, Germany), Quantarray (GSI Lumonics) . Direct, non-imaging methods can also be used as an alternative.

In the preparation of individual arrays in series, each chip can be visually recorded individually. If the preparation of the array takes place in parallel, e.g. on a wafer scale, the occupation density can be determined alternatively for each chip individually or in parallel by means of a high resolution procedure over the entire wafer.

According to the invention, the analysis of the occupation density is used to identify arrays which are unsuitable, or suitable only to a limited extent, for performing experiments. These include for example:
Array elements with too little occupation density;
Array elements with an excessively high deviation of the occupation density in comparison with the average (to be expected) occupation density of this array element on the arrays produced;
Array elements with a non-homogeneous occupation density on the array;
Array elements without signal.

These values are preferably used in order to discard arrays or to label non-usable array elements. The results of the quality control can be saved e.g. in a database such as Partisan (Clondiag), if necessary with other data relevant for the preparation and use of arrays. Discarded arrays can be marked by a flag and thus blocked for use.

The evaluation of the occupation density is preferably carried out in a computer-aided and/or automated manner. Threshold values for discarding arrays and/or labelling of unsuitable array elements depend on the desired use of the arrays.

In a further essential aspect of the present invention, a method is provided for the qualitative and/or quantitative detection of targets from a sample by molecular interaction between probes and targets on probe arrays, which method comprises the following steps:
a) Providing a probe array according to the invention as describe above;
b) optionally, detection of the probe molecules immobilised on the array surface, in the form of signal intensities;
c) incubating the probe array with the sample to be analysed;
d) optionally, washing under conditions, under which a specific interaction between the target molecule and the probe molecule remains largely stable and unspecifically bound targets are removed;
e) optionally, again detection in the form of signal intensities;
f) selective cleavage of the selectively cleavable bond in the probe molecules;
g) optionally, washing in order to remove labelled probe molecule fragments which have not been retained at the array surface by interaction with target molecules;
h) Detection of the labelled probe molecule fragments remaining on the array surface by interaction with target molecules in the form of signal intensities; and
i) optionally, standardisation of the signal intensities detected in step h).

In comparison with the methods hitherto known for the detection of probe/target interaction on probe arrays, the method according to the invention provides two major advantages.

For one thing there is no longer any need to label the targets. Consequently, all errors and variations described above which may arise in the course of the labelling of the target, are completely avoided.

For another thing the array elements of the inventive array are provided with a label, or a detectable unit. The quality of the array can thus be determined as described above, before the detection experiment, at the level of the individual array elements. The signal intensities thus obtained for the individual array element, which correspond to the occupation density for the individual array element, can be used to normalise the experimental results, i.e. the signal intensities obtained after the probe/target interaction.

In this way, the essential causes of inaccuracies in array-based assays can be eliminated by the detection method according to the invention.

Usually, the quality of the array is controlled directly after production so that the occupation densities required for the standardisation are already available to the user of the method according to the invention. In this case, step b) of the method according to the invention is not required. If desired, the quality of the array provided can be checked again, or for the first time, using the signal intensities optionally detected in step b) at the time of use of the array, also as part of the detection method according to the invention.

Some further embodiments are described below, by means of which a standardisation or normalisation of the results is ensured. Within the scope of the present invention, normalisation or standardisation means that comparability of the signal intensities detected on a probe array is ensured, whereas standardisation means that the comparability of experiments carried out on different arrays is ensured.

The normalisation of the array experiments is based on the particular properties described above of the array according to the invention. In this connection, the construction of the array according to the invention provides various possibilities for the evaluation of array experiments. The degree of standardisation can be adapted to the requirements of the concrete experiment, or detection method, and will be higher in quantitative analyses than in analyses where yes/no predictions are to be obtained.

In one embodiment, signal intensities Sₒ, which represent a measure of the occupation density of the array element concerned, are determined for each array element during the quality control of the array, the signal intensities representing a measure of the occupation density of the array element concerned. These signal intensities are mathematically converted into correction factors kₐ, which are used for the normalisation of the signal intensity measured on each individual array element after the completion of the experiment.

Preferably, kₐ is determined by determining the mean of the signal intensities Sₒ of all the array elements and dividing the average signal intensity of all array elements by the signal intensity Sₒ of each array element. Alternatively, kₐ is determined by establishing another mathematical combination from the signal intensity Sₒ of the array elements before the experiment.

According to the invention, the correction factor kₐ can be used to correct the variations in the signal intensity S₁ caused by the different occupation density of the array elements after interaction with the target, the cutting of the labile bond and the optional washing have taken place. The corrected signal intensity is referred to as S_{2.}

In the simplest case, S₂ is calculated from S₁ by a mathematical combination with kₐ. Preferably, the mathematical combination is a multiplication. Particularly preferably, the type of mathematical combination is experimentally optimised for the respective array type and the respective sample type.

In one embodiment of the detection method according to the invention, the standardization in step i) is thus carried out by mathematical combination of the signal intensities obtained in step h) with a correction factor, which can be determined by means of the signal intensities obtained during the quality test in step b).

Further factors, apart from kₐ, can be taken into account in the calculation of S₂. For example, a standardisation against experimental variations which may occur from the beginning of the incubation of sample and array can be made by means of array elements distributed over the surface of the array, on which elements the control probes, as described above, are arranged. Hereinafter such array elements will also be referred to as control elements. In the case of oligonucleotide probes used as probe molecules, for example, after hybridisation with the target having a- sequence complementary to the control oligonucleotide probe, cleavage of the labile bond and, optionally, after washing steps, the signal intensity is determined on these control elements and, optionally, normalised by mathematical combination with kₐ.

The signal intensities normalised, optionally, by mathematical combination with kₐ, should be the same for all control elements. In the case of strong deviations, a control factor kₑ is calculated for each array element, which allows normalisation against experimental variations leading to differently bright areas on the array. The correction factor kₑ is calculated from the deviation of the signal intensities, optionally normalised by mathematical combination with Kₐ, of the control elements adjacent to an array element, whereby the distance between the array element and the control element is considered. The correction factor is preferably calculated according to the algorithm described on page 1267 of Selinger D. W., Cheung K. J., Mei R., Johansson E. M., Richmond C. S., Blattner F. R., Lockhart D. J., Church G. M., Nat. Biotechnol. 2000 Dec;18(12).

Preferably, S₂ is calculated from S₁ by mathematical combination of kₐ and kₑ. Alternatively S₂ is calculated from S₁ by mathematical combination with kₑ.

In a further embodiment of the detection method according to the invention, standardization consequently takes place by mathematical combination of the signal intensities obtained in step h) with a correction factor determined on the basis of the signal intensities during the quality test and/or a correction factor determined by way of the signal intensities of control elements.

The correction against the background signal represents a further embodiment of the standardised evaluation. For this purpose, array elements are used on which probes are arranged which undergo no or no detectable interaction with targets from the sample. In the following, these array elements will also be referred to as background elements. Following hybridisation, cleavage of the labile bond and, optionally, washing steps, the signal intensity of these background elements is measured, normalised, if necessary by mathematical combination with kₐ and/or kₑ and subtracted from the signal intensities of all array elements.

Preferably, the signal intensity of the background elements is subtracted from S₁. S₁′ is the result which can be corrected, according to one of the embodiments described above, with kₐ, kₑ or kₐ) and kₑ such that the corrected signal intensity S₂′ is obtained.

Also preferably, the corrected signal intensity S₂′ is obtained by subtracting the signal intensity of the background elements from S₂.

In a further embodiment of the detection method according to the invention, standardization is made by subtracting the detected signal intensities of background elements from the signal intensities obtained in step h) and corrected, if necessary, as described above.

Particularly in the case of quantitative analyses a normalisation of the results relative to the characteristic curve of the assay, i.e. the dependence of the signal intensity on the quantity of detectable units present, is advantageous. For this normalisation, probe arrays are used, on which probes are arranged on at least one, preferably several array elements, wherein the probes are labelled but not provided with a selectively cleavable bond and wherein the probes preferably differ in their degree of labelling in a characteristic manner, for example with a defined mixture of labelled and unlabelled probes varying in the form of a dilution series from array element to array element. Such array elements are hereinafter referred to as detection standard elements.

Standardization of the detection is thus preferably carried out by comparing the signal intensities obtained for an array element with the signal intensities of array elements on which oligonucleotide probes not provided with a selectively cleavable bond and/or detectable units coupled directly with the array surface are arranged.

On completion of such an experiment, the signal intensities of the corresponding detection standard elements are determined and, optionally, normalised by subtracting the background signal and correction with the correction factors described above. The values of the detection standard elements, which optionally have been normalised, are plotted against the mixing ratio of labelled and unlabelled substance. This results in a calibration curve which indicates the dynamic range and the type of interdependence between the signal intensity and the quantity of detectable units, the so-called characteristic curve of the array.

The characteristic curve can be used, on the one hand, to identify non-quantifiable values which are outside the dynamic area and, optionally, to exclude them from the analysis. On the other hand, a certain quantity of detectable units can be assigned to all other signal intensities. This provides the possibility of a quantitative analysis as well as the comparison of experiments carried out with different arrays. Depending on the requirements, methods can be selected among the normalisation and standardisation methods described above and carried out individually or in any desired combinations.

Consequently, the method according to the invention is not only a method for the detection of interactions on probe arrays where the labelling of target molecules can be omitted, but rather a method consisting of processing steps for the preparation and quality monitoring of probe arrays suitable for such a detection, of processing steps for the utilisation of this array in assays as well as of processing steps for the evaluation and standardization of the results.

In one preferred embodiment, the incubation of the probe array takes place with a sample consisting of unlabelled targets. For particular applications, e.g. for purposes of the calibration of conventional detection methods as compared to the method according to the invention, a labelled target mixture can also be used.

If necessary, the unlabelled targets are fragmented by a suitable enzymatic, physical and/or chemical process before incubation with the probe array.

According to a preferred embodiment of the method according to the invention, the interaction between the target and the probe is a hybridisation between two nucleotide sequences. The hybridisation of the target with the probes arranged on a probe array takes place according to any one of the known standard protocols (compare Lottspeich and Zorbas, 1998, among others). For example, the incubation of the sample and the array takes place in an aqueous hybridisation buffer or a hybridisation buffer containing formamide. Preferably, aqueous hybridisation buffers based on SSC, SSPE or phosphate buffer, which particularly preferably contain sodium chloride or sodium nitrate, are used.

The resulting hybrids can be stabilised by a covalent binding, for instance by psoralene intercalation and subsequent cross-linking, or, as described in U.S. Pat. No. 4,599,303, by non-covalent binding, e.g. by binding intercalators.

Following the hybridisation of the targets with the labelled probes arranged on a probe array, a washing step is usually carried out by means of which unspecific and thus less strongly bound components are removed. Since the targets are not labelled in the case of the method according to the invention, the washing step can alternatively be omitted, because unspecifically bound targets do not stabilise the cleaved probe/target complex. The washing step which may possibly be necessary after the interaction of the array and the target can also be carried out in an SSC, SSPE or phosphate-based or other suitable buffer systems familiar to the persons skilled in the art under conditions which destabilise unspecific interactions, whereas specific interactions remain relatively unaffected.

Alternatively, the interaction between the target and the probe is a reaction between an antigen structure and the corresponding antibody or a hypervariable segment thereof or a reaction between a receptor and a corresponding ligand. If the probe molecule is a polypeptide, the selective modification of an amide bond is a suitable means for providing a selectively cleavable bond.

The interaction of the targets with the probes, or the binding or recognition of the target by specific probes is usually a spontaneous, non-covalent reaction under optimum conditions. This also comprises non-covalent chemical bonds. The composition of the medium and further chemical and physical factors influence the rate and the strength of the binding. Thus, a higher stringency during the nucleic acid recognition, for example, reduces the kinetics and strength of the binding between two not absolutely complementary strands. An optimisation of the binding conditions is also required for the antigen/antibody or the ligand/receptor interaction; however, the binding conditions are usually less specific.

In the case of the process according to the invention, the cleavage of the labile bond usually takes place under conditions under which both the uncleaved hybrid and the hybrid, which is present after cleavage and less stable as a result of the single strand breakage, are retained. Such conditions are provided in particular by a high ionic strength and/or a low temperature.

Preferably, the selectively cleavable bond is cleaved selectively by chemical and/or physical methods, particularly preferably by the addition of ions such as acid anions, base cations, fluoride ions and/or heavy metal ions such as mercury ions and/or silver ions.

If the bond to be cleaved is a phosphothioate bond bridging two nucleosides, this bond can be cleaved by oxidative attack by iodine and/or by heavy metal ions such as silver ions or mercury ions. Preferably, the cleavage of the bond is performed with silver ions, particularly preferably with silver nitrate. The solution used for the cleavage preferably has a high ion concentration in order to stabilise the hybrids.

The ionic strength required to stabilise the hybrids is preferably achieved by salts which do not form poorly soluble salts with silver ions. Sodium nitrate is particularly preferred. The final concentration of salt for the increase of the ionic strength, particularly of sodium nitrate, in the cleavage buffer ranges from a saturated solution to 10 mM, preferably from 100 mM to 2 M and particularly preferably from 500 mM to 1M.

Preferably, the cleavage reaction is performed at temperatures of from −70° C. to 100° C., particularly preferably from −20° C. to 50° C., and most preferably from 0° C. to 20° C.

The concentration of silver nitrate in the cleavage solution particularly ranges from a saturated solution up to 1 μM. Preferably, a silver nitrate solution at a concentration of between 1 M and 5 mM, particularly preferably between 10 and 50 mM is used.

If the bond to be cleaved is a bridged phosphothioate and if this bond is cleaved with silver ions, ions which form a poorly soluble product with silver ought to be preferably removed before the cleavage reaction. This is achieved, for example by washing with a buffer, which does not contain such ions, preferably by washing with 5 M to 10 mM sodium nitrate, particularly preferably by washing 1 M sodium nitrate.

In an alternative embodiment of the detection method according to the invention, step g) i.e. the washing step for removing labelled probe molecule fragments not retained on the array surface by interaction with target molecules, can be omitted. A precondition for such a homogeneous assay without corresponding washing is the use of suitable measuring techniques known to the persons skilled in the art such as e.g. fluorescent polarisation measurements which allow a clear distinction between the signals of bound and unbound labels. A further alternative in the performance of such an assay without the washing step g) is the use of confocal optical structures, which exclusively detect the signal on the surface.

The detection method according to the invention described by way of the example of probe arrays can obviously also be used for other non-array-based methods, in which the probe molecules described above comprising at least one label and at least one selectively cleavable bond are used.

An application of the detection principle according to the invention for microtiter plate assays and assays in reaction vessels, the so-called tube assays, is hereinafter described. The immobilisation and synthesis of the probe molecules described above on the particular carrier system is performed analogously to the manufacturing process of the probe arrays according to the invention. Any differences in the procedure which arise from the use e.g. of microtiter plates or reaction vessels instead of array substrates are of course familiar to the person skilled in the art.

The use of probes immobilised on the microtiter plate, or on the vessel's bottom, or at the vessel's wall with selectively cleavable bonds, or units in the microtiter plates and reaction vessels can be subdivided into two embodiments.

On the one hand the detection of the interaction of a probe which is labelled, as described above, e.g. with a dye, a gold particle, a latex bead and such like, can take place with a target in a manner analogous to the procedure described above by way of the example for probe arrays by detection of the labels remaining on the surface of the vessel after the cleavage reaction.

In this embodiment, the detection can take place following the completed washing by measuring the label-specific signal, e.g. fluorescence in the case of the corresponding dye. A homogeneous assay without corresponding washing is also possible. A precondition for this is that suitable measurement techniques such as fluorescent polarisation measurements are used which allow a clear distinction to be made between the signal of bound and unbound labels. A further alternative in carrying out such an assay is the use of confocal optical structures which exclusively determine the signal on the surface. Several label-specific methods have already been discussed in the state of the art and might be used also in microtiter plates or vessel-based assays, in analogy to the array-based assays.

On the other hand, the detection of the interaction of a probe with a target can occur by detecting the labels present in solution after the cleavage reaction. In this embodiment, the detection in solution provides a target-specific signal if only one target is to be analysed in the vessel, or the different targets to be analysed are each detected with differently labelled probes, e.g. when four dyes are used for four probes.

The detection in solution provides the advantage of being able to guarantee a sensitive measurement with very simple constructs, and is above all useful when it can be assumed that the differences in the binding of targets against the surface are only minimal, so that a comparative measurement of the immobilised signal would provide a strongly positive signal and the minimal changes in the signal are not of significant importance. Homogeneous assays can also be realized in this way if a detection method is selected which allows to discriminate between bound and unbound signals, for instance confocal correlation spectroscopy or fluorescent polarisation measurements.

Dispensing with the labelling of the target and the possibility of carrying out homogeneous assays are essential advantages of the method and also speak for their use e.g. in assays on microtiter plates and in reaction vessels.

A further subject matter of the present invention is a kit for the qualitative and/or quantitative detection of targets from a sample by molecular interaction between probes and targets on probe arrays, comprising the following components:
a) A probe array according to the invention;
b) Reagents for the selective cleavage of the selectively cleavable bond in the probes;
c) Hybridisation buffer; and
d) optionally, washing buffer.

Preferably, the reagents are selected from the group consisting of heavy metal ions, e.g. mercury ions and/or silver ions.

In preferred embodiments of the kit, the probe array according to the invention, additionally comprises a reaction chamber, and/or a detection device, and/or a temperature control unit, and/or a light source.

The detection device is preferably selected from the group consisting of a fluorescence microscope, a laser scanner for arrays, a microscope and a CCD-based scanner and usually records the entire range of the probe array.

Preferably, a light source is selected from the group consisting of a laser, laser diodes and a source of white light with a corresponding filter.

In a particularly preferred embodiment, the probe array and a reaction chamber and/or a detection device and/or a temperature control unit and/or a source of light are in the form of a highly integrated autonomous unit.

The probe array according to the invention and/or the detection method according to the invention and/or the kit according to the invention can be used for the qualitative and/or quantitative detection of targets from a sample by molecular interaction between the probes and the targets, in particular for the analysis of the genotypic state and/or the physiological state of cells.

Within the scope of the present invention, a probe array is provided where oligonucleotides are immobilised and/or synthesised site-specifically, which oligonucleotides carry a label opposite to the immobilisation position. Between the immobilisation and the labelling position, a labile bond, e.g. a bridged phosphothioate bond, is preferably present approximately in the centre.

The probe array according to the invention is incubated with a sample under conditions allowing a specific interaction, e.g. a hybridisation. Targets with an affinity to the probes of the probe array will bind to them. If necessary, steps are taken to remove the unspecifically bound targets. Preferably, the distribution of the labels on the surface is documented to test the quality of the probe array produced using an imaging procedure. Subsequently, the labile bond is cut e.g. by the addition of silver ions. This takes place under conditions at which the specific interaction between the target and the probe is sufficiently stable to guarantee that the labelling is retained also after cleavage by the interaction between the target and the probe. Probes on which no specific interaction or no interaction at all takes place with targets will lose their label as a result of the cleavage. The result of the conversion is preferably documented by an imaging procedure. The presence or absence of targets in the sample can be deduced from the labelling intensity of the individual probes after the bond has been cleaved.

Due to the fact that labelling of the target molecules is no longer required, considerable time and cost savings can be achieved by the probe array according to the invention. Moreover, labelling is no longer dependent on the nature of the unknown target molecules such that the detection results become comparable. A further advantage is the fact that labelling of the probes on the probe array can be easily standardized, and especially can also be automated. Moreover, the labelled probes simultaneously also represent a quality control for the synthesis and immobilisation of the probes on the probe array. Finally, labelling of probes instead of targets also guarantees a standardisation of the image evaluation.

The following examples and figures serve to illustrate the invention and should not be interpreted as restrictive.

### EXAMPLES

### Example 1

### Synthesis of 5′-S-(dimethoxytrityl)-mercapto-5′-deoxythymidine-3′-O-(2-cyanoethyl, N,N′-diisopropyl phosphite)

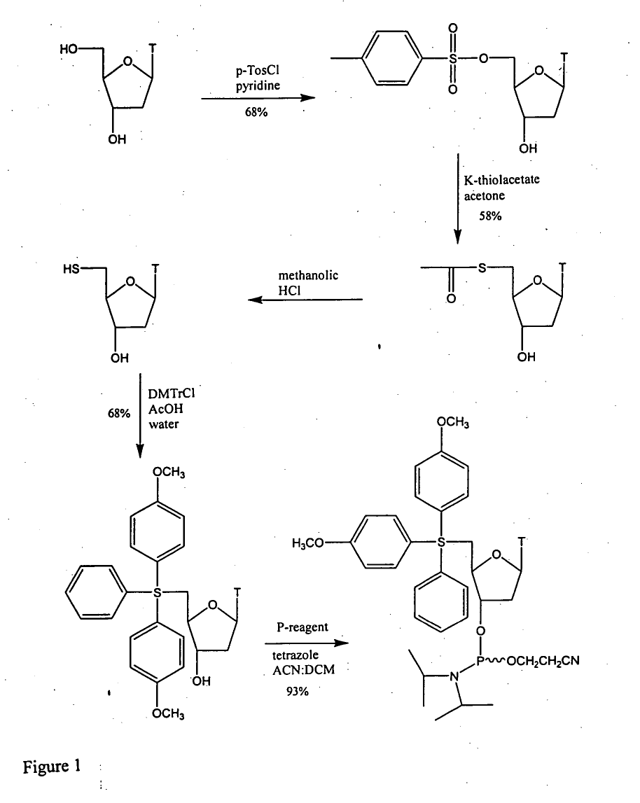

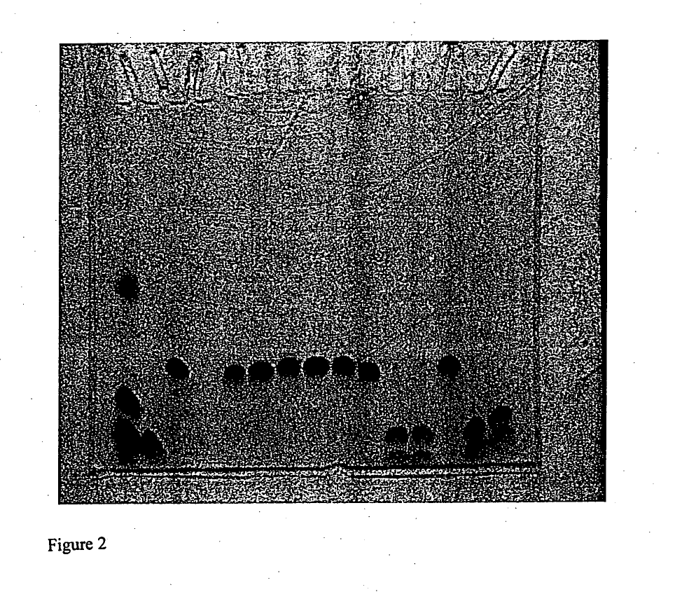

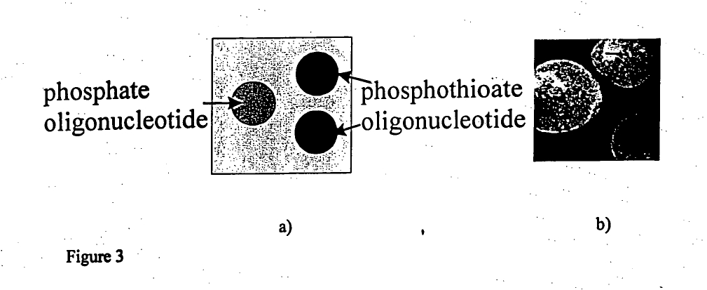

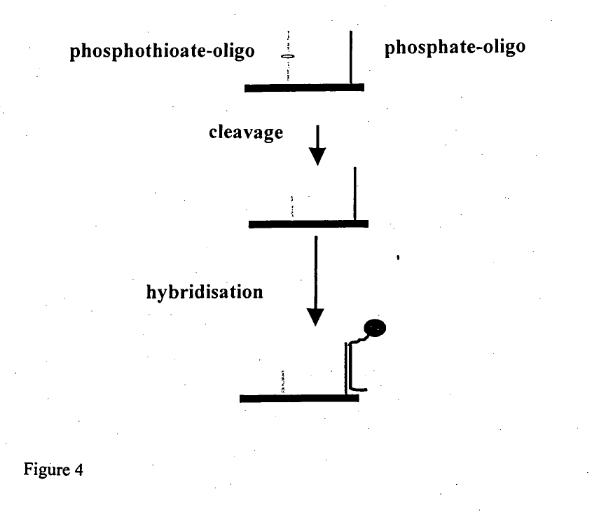

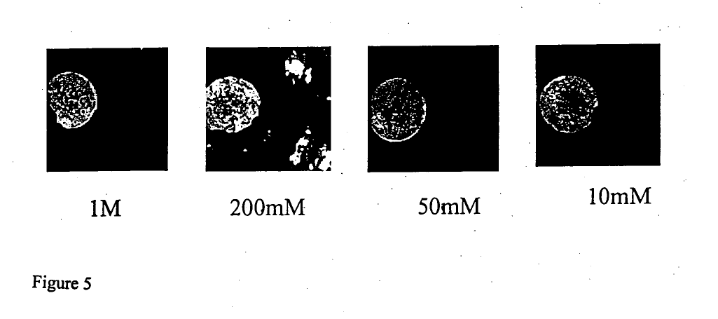

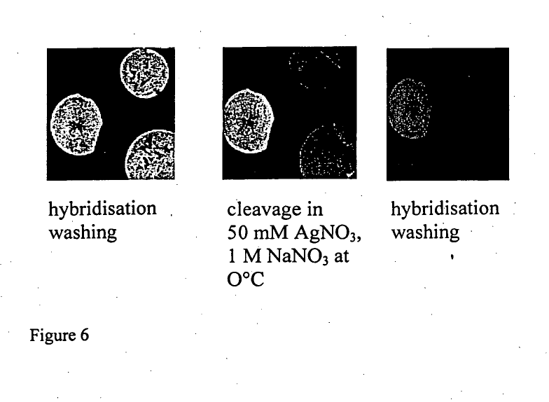

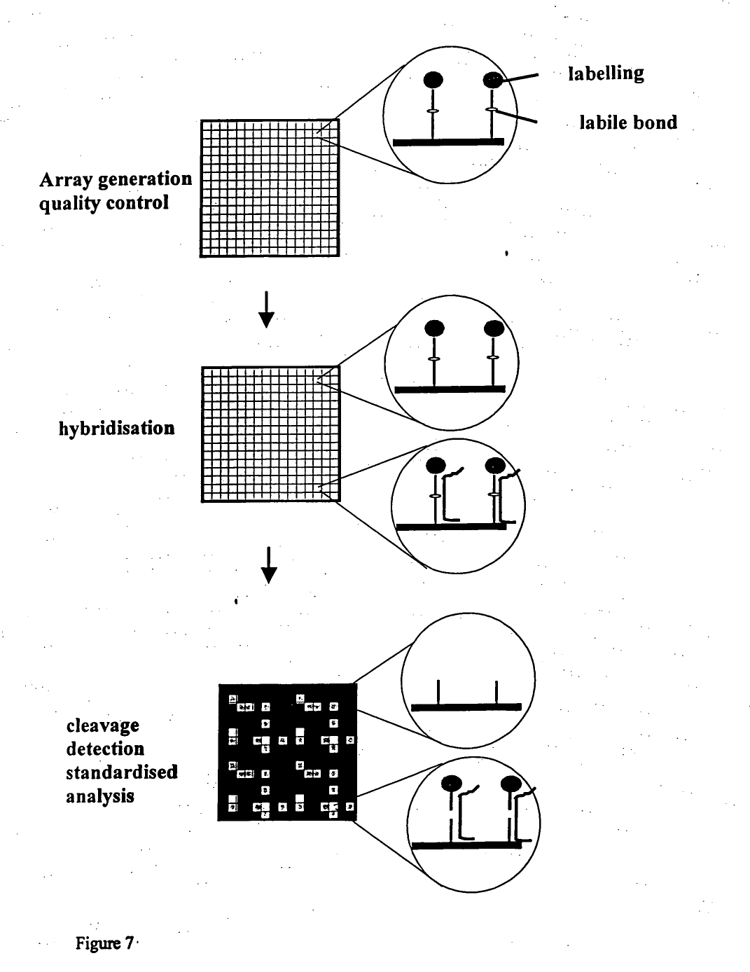

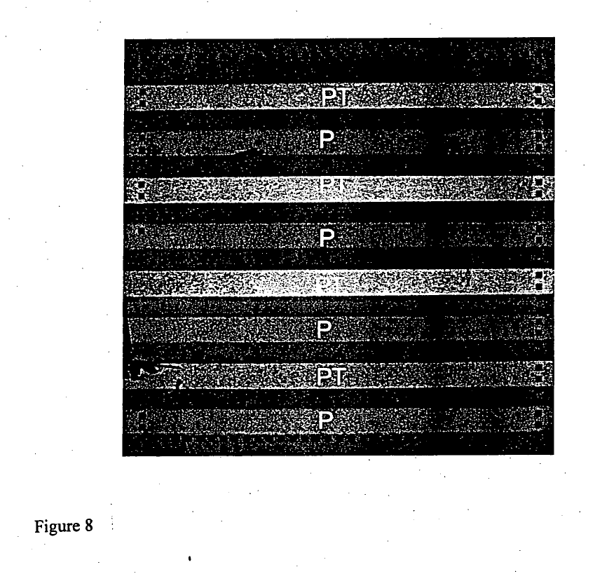

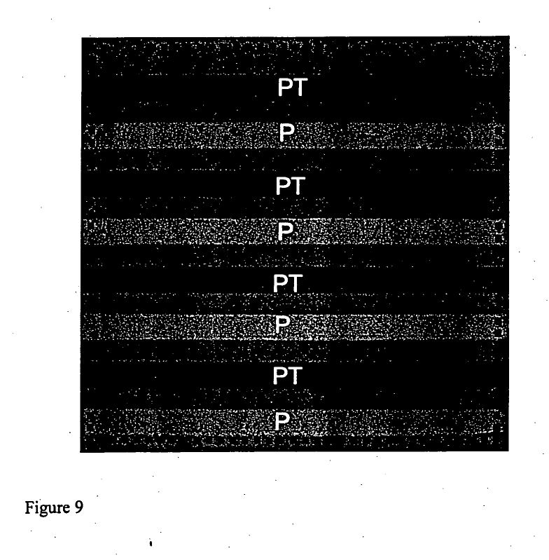

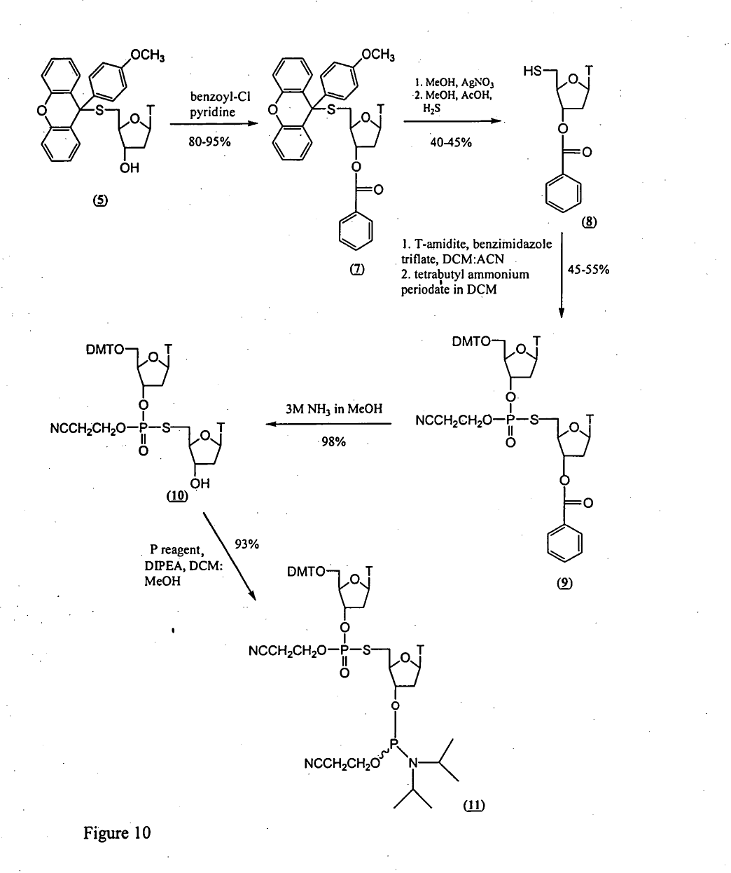

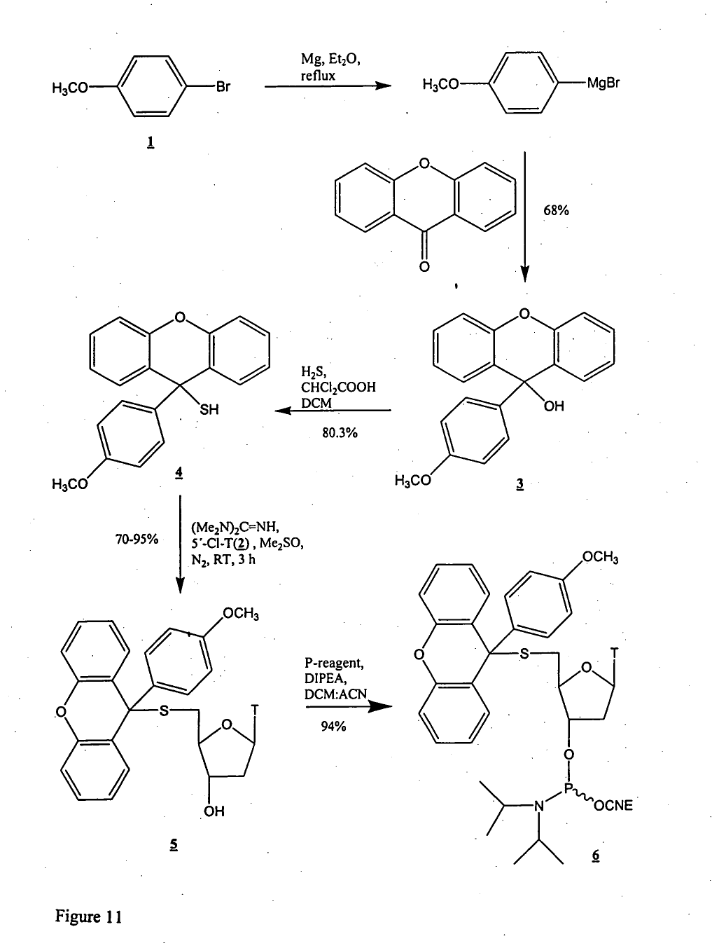

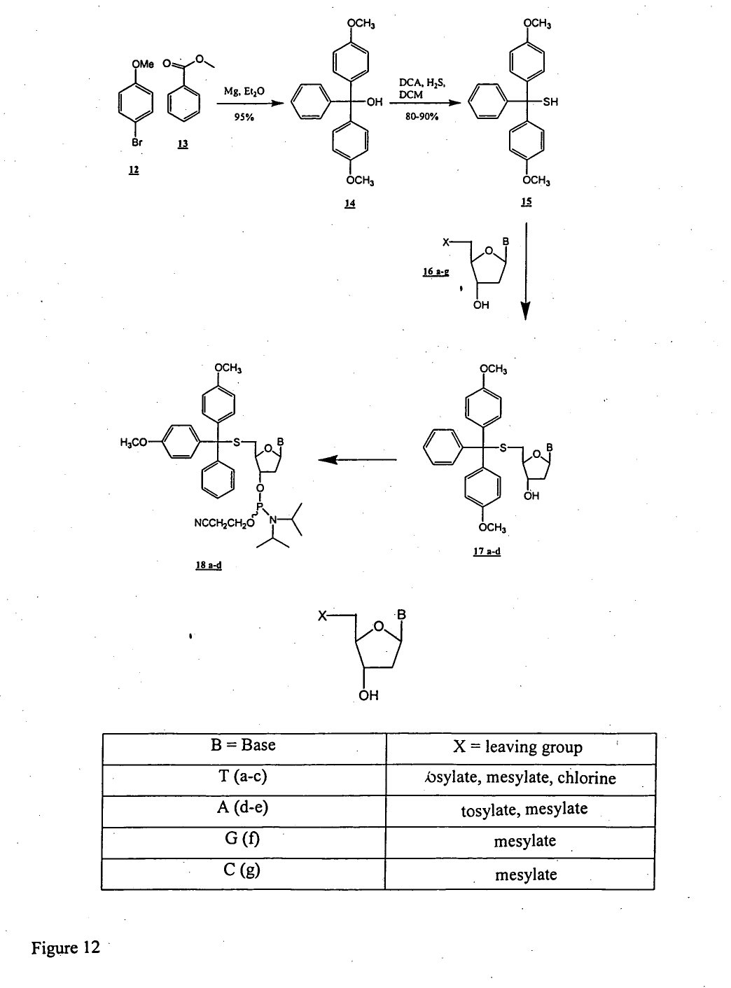

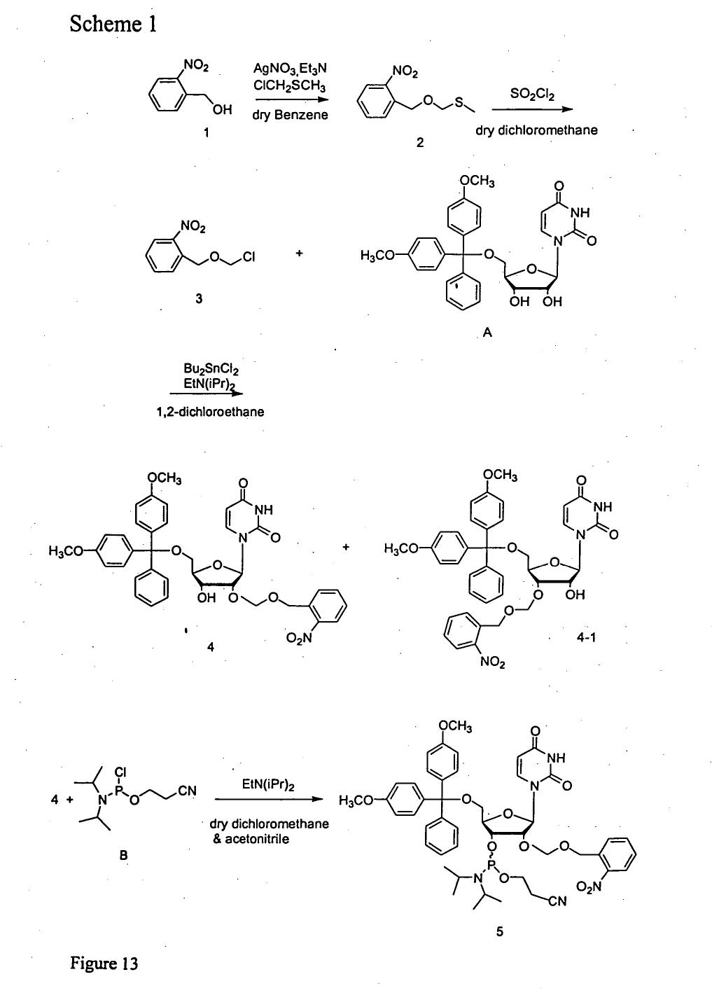

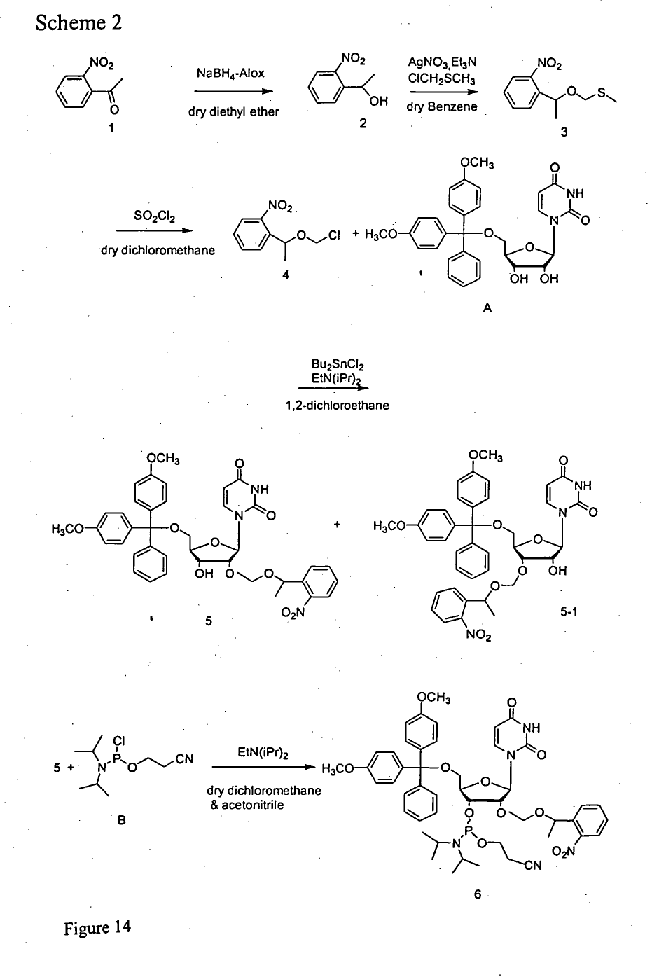

The synthesis of the phosphoramidite building block according to Example 1 is performed in five steps according to the following synthesis scheme (compare also FIG. 1):

Thymidine is suspended in pyridine and cooled to 0° C. Over a period of 30 min, a solution of p-toluenesulfonyl chloride in pyridine is added dropwise and the solution is stirred overnight at 4° C. The solution is poured into iced water and the resultant precipitate is filtered with suction and then dissolved in dichloromethane and extracted twice with 5% monosodium carbonate solution and once with saturated sodium chloride solution. The organic phase is dried on sodium sulfate and treated in the rotary evaporator. The yield is 68%.

5′-O-(p-tolyl sulfonyl)-thymidine and potassium thioacetate are suspended in acetone and heated for 3 h to 50° C. under an argon atmosphere. The suspension is then stirred for 16 h at room temperature. The precipitate is filtered with suction and washed again with a small amount of acetone. The solution is treated in the rotary evaporator and the residue is subsequently purified by column chromatography (solvent: dichloromethane: methanol 9:1). The yield is 58%.

5′-S-(acetyl)-thymidine is dissolved in methanol and 5 molar methanolic hydrochloric acid are added such that the final concentration of acid is 1 molar. The solution is heated to 45° C. under argon atmosphere and stirred for 2 h. The solution is reduced to approximately half its volume at the rotary evaporator and added to a solution of DMTr-Cl in acetic acid and water. The solution is stirred for 3 h at room temperature and then again reduced to half its volume in the rotary evaporator. The solution is diluted with water and adjusted to a pH 10 using 2 molar caustic soda solution. Dichloromethane is added and extracted twice with 5% mono-sodium carbonate solution and once with saturated sodium chloride solution. The organic phase is dried over sodium sulfate and treated in the rotary evaporator. The residue is purified by column chromatography (solvent dichloromethane:methanol 97:3). The yield of 5′-S-(dimethoxytrityl)-thymidine is 68%.

To a solution of 5′-S-(dimethoxytrityl)-thymidine in dichloromethane:acetonitrile, tetrazol is added under argon atmosphere and subsequently phosphorus bis(diisopropyl amide)-2-cyanoethyl ester is slowly added dropwise. After 2 hours, the reaction is quenched by the addition of n-butanol. The solution is diluted with acetic acid ethyl ester and extracted twice with 5% monosodium carbonate solution and 1× with saturated sodium chloride solution. The organic phase is dried over sodium sulfate and treated in the rotary evaporator.

The residue is taken up in some dichloromethane diethyl ether and pipetted into cold n-pentane. The yield of 5′-S-(dimethoxytrityl)-mercapto-5′-deoxythymidine-3′-O-(2-cyanoethyl, N,N′-diisopropyl-phosphite) is 93%.

The amidite thus produced is subsequently incorporated into a model oligonucleotide (24 mer, compare example 2).

a) Characterisation of the compounds produced
5′-O-(p-tolyl sulfonyl)-thymidine (C₁₇H₂₀N₂O₇S)

| ESI(+) MS | 396.42 g/mole | |
|---|---|---|
| calculated mass: | | |
| found mass: | 397.1 g/mole | |
| Melting point | 168-169° C. | |
| Rf value | dichloromethane:methanol 9:1 | 0.39 |
| ¹H-NMR | DMSO | |

1.75(s, 3H, CH₃-T); 2.10(m, 2H, 2′,2″-H);
2.40(s, 3H, CH₃-tosyl); 3.85(m, 1H, 4′-H);
4.21(m, 3H, 5′,5″-H, 3′-H); 5.57(d, 1H, 3′-
OH);
6.14(t, 1H, 1′-H); 7.36(d, 1H, 6-H); 7.44(d,
2H, AA′BB′); 7.82(d, 2H, AA′BB′); 11.31(s,
1H, NH)

5′-S-acetyl-thymidine (C₁₂H₁₆N₂O₅S)

| ESI(+) MS | 300.33 g/mol | |
|---|---|---|
| calculated mass: | | |
| found mass: | 301.2 g/mol | |
| Rf value | dichloromethane:methanol 9:1 | 0.37 |
| ¹H-NMR | DMSO | |

1.81(s, 3H, CH₃-T); 2.07(m, 1H, 2′-H); 2.23(m,
1H, 2″-H); 2.37(s, 3H, O-Ac); 3.1(m, 1H,
5′-H); 3.23(m, 1H, 5″-H); 3.75(m, 1H, 4′-H);
4.1(m, 1H, 3′-H); 5.41(d, 1H, 3′-OH); 6.15(t,
1H, 1′-H); 7.45(d, 1H, 6-H); 11.03(s, 1H, NH)

5′-S-(dimethoxytrityl)-thymidine (C₃₁H₃₂N₂O₆S)

| ESI(−) MS | 560.7 g/mol | |
|---|---|---|
| calculated mass: | | |
| found mass: | 560.2 g/mol | |
| Rf value | dichloromethane:methanol 9:1 | 0.51 |
| ¹H-NMR | CDCl₃ | |

1.86(s, 3H, CH₃-T); 2.06(m, 1H, 2′-H); 2.29(m,
1H, 2″-H); 2.5(m, 1H, 5′-H); 2.54(m, 1H,
5″-H); 3.78(m, 7H, 4′-H, OCH₃); 4.1(m, 1H,
3′-H); 6.16(t, 1H, 1′-H); 6.83(m, 5H, 6-H,
aromatic); 7.22-7.43(m, 9H, aromatic)

5′-S-(dimethoxytrityl)-mercapto-5′-deoxythymidine-3′-O-(2-
cyanoethyl,N,N′-diisopropyl phosphite) (C₄₀H₄₉N₄O₇PS)

| ESI(+) MS | 760.86 g/mol | |
|---|---|---|
| calculated mass: | | |
| found mass: | 761.2 g/mol | |
| Rf value | dichloromethane:methanol 9:1 | 0.65 |
| ³¹P-NMR | CDCl₃ + 0.1% DIPEA | |
| | 149.55, 149.35 | |

### Example 2

### Synthesis of a Model Oligonucleotide 5′-AGC CCT TAC TTT GAC GGT ATA TCT-3′

The synthesis of the oligonucleotide is performed according to the phosphodiester method. Couplings of the unmodified building blocks take place according to the standard protocol on a DNA synthesizer (PerSeptive Biosystem or Applied Biosystem, Weiterstadt, Germany). The modified building block (underlined) is coupled on a DNA synthesizer (Applied Biosystem) according to a modified synthesis protocol. Further synthesis is also carried out on a DNA synthesizer (PerSeptive Biosystem).

Here, coupling of the modified building block is carried out by adding, twice, the amidite and tetrazol to the column (double couple method) and the prolongation of the coupling time to 300 s. Subsequent capping and oxidation take place under standard conditions. The cleavage time for the detritylation must be prolonged five-fold and detritylation is followed directly by rinsing of the column with a DTT solution (220 mM DTT (1,4-dithiothreitol)) in a solution of THF/pyridine/water 7/1/2). Rinsing with DTT solution is carried out according to the following protocol:
a. 30 s addition of DTT solution to the column
b. 30 s wait step
c. 30 s addition of DTT solution to the column
d. 150 s wait step
e. 30 s addition of DTT solution to the column
f. 150 s wait step
g. 30 s addition of DTT solution to the column
h. 150 s wait step
i. 30 s addition of DTT solution to the column
j. 150 s wait step
k. 30 s addition of DTT solution to the column

The DTT solution is rinsed from the column in a washing step which corresponds to the standard protocol following iodine oxidation. However, the washing and rinsing steps have been doubled in this case. The remainder of the synthesis after the incorporation of the modified building block takes place under standard conditions. The synthesis scale is 1 μmole.

The oligonucleotide is treated with ammonia overnight, which leads to the cleavage of the oligonucleotide from the solid phase and the deprotection of the base protection groups. For the subsequent purification, the ammonia solution is injected directly into a R3-HPLC column (PerSeptive Biosystem) (gradient 0-25%, buffer B in 10 min, buffer A 0.1 molar TEAA solution; buffer B ACN). The product fractions are collected and the solvent is removed and the oligonucleotide is then precipitated as sodium salt by ethanol precipitation. An O.D. determination of the dried precipitate is subsequently carried out. HPL chromatography, gel electrophoresis and a MALDI-MS investigation are used for the analysis.

In order to be able to detect the cleavage to be carried out later on, the cleavage fragments thus formed and a control (unmodified model sequence) are synthesised as further oligonucleotides. The synthesis of the oligonucleotides is performed on the PerSeptive Biosystem synthesizer according to the standard protocol for a 1 μmole synthesis. The cleavage and deprotection is done overnight with ammonia and the subsequent purification via HPL chromatography. The oligonucleotides are precipitated as Na salt and again analysed by HPL chromatography, gel electrophoresis and MALDI-MS. They are used as controls for the subsequent cleavage experiments.

### Example 3

### Cleavage of the Model Oligonucleotide

The cleavage of the model oligonucleotide is performed by the addition of silver nitrate. The experiment is carried out as follows:

15 μl of a 50 mM silver nitrate solution are added to 1 O.D. of the model oligonucleotide and the solution is allowed to stand for 1 h at room temperature. The reaction is quenched by adding 4 μl of a 220 mM DTT solution. After 1 h, the sample is centrifuged and the solution is removed. HPL chromatography and gel electrophoresis (15% TBE/urea gel, 1.0 mm×15 well, 250 V, 90 min) follows for analytical purposes. The fragments formed during the cleavage

| | |
|---|---|
| 5′-AGC CCT TAC T-3′ | (10mer) |
| 5′-HO-TT GAC GGT ATA TCT-3′ | (14mer) |

and the unmodified oligonucleotide

5′-AGC CCT TAC TTT GAC GGT ATA TCT-3′

The HPL chromatograms are taken on a Beckman HPLC system (Gold system).

The following buffers were used:

| | | |
|---|---|---|
| Buffer A | Acetonitrile | 400 ml |
| | Water | 1600 ml |
| | NaH₂PO₄ × 2H₂O | 3.12 g |
| Buffer B | Acetonitrile | 400 ml |
| | Water | 1600 ml |
| | NaH₂PO₄ × 2H₂O | 3.12 g |
| | NaCl | 175.32 g |

The following gradient was used on the ion exchange column (Waters):

| | |
|---|---|
| 0 min: | 5% buffer B |
| 40 min: | 60% buffer B |

The retention times of the oligonucleotides are summarised in the following Table:

| | |
|---|---|
| 5′-TTG ACG GTA TAT CT-3′ | 24.26 min |
| 5′-AGC CCT TAC T-3′ | 23.22 min |
| 5′-AGC CCT TAC TTT GAC GGT ATA | 28.42 min |
| TCT-3′ | |
| Cleavage reaction carried out | 23.49 min; |
| | 24.65 min |

The HPL chromatogram and the gel pattern (compare FIG. 2) indicate that the cleavage of the modified oligonucleotide is quantitative. After the cleavage reaction, only the 10 mer and the 14 mer can be detected. No modified oligonucleotide can be detected, whereas the unmodified oligonucleotide is not cut, and can be detected. Further experiments show that the cleavage with a 10 mM silver nitrate solution is carried out quantitatively within 5 min.

As a further analytical procedure, a measurement was run on a Biacore device (Uppsala, Sweden) in order to indicate that the cleavage is possible also on a solid phase. The following oligonucleotide was synthesised for these measurements:

5′-biotin-AGC CCT TAG TTT GAC GGT ATA TCT-3′

The 5′-end of the oligonucleotide was biotinylated (Glen Research, Sterling, Va., USA). The synthesis of the oligonucleotide is performed as described in Example 2. Further modification at the 5′-end was carried out on an Applied Biosystem 394 synthesizer. For the synthesis, a standard cycle was applied whose coupling time was extended to 300 s. Purification and processing also took place as described in Example 2. Moreover, three oligonucleotides were synthesised for the measurements, which exhibit different complementary regions with the modified model oligonucleotide. These three compounds, which were synthesised and purified according to standard conditions, are as follows:

| | |
|---|---|
| 1. | 5′-GCA GCT AGA TAT ACC GTC AA-3′ |
| 2. | 5′-GCT AGA TAT ACC GTC AAA GT-3′ |
| 3. | 5′-GAT ATA CCG TCA AAG TAA GG-3′ |
| | 5′-AGC CCT TAC TTT GAC GGT ATA TCT-3′ |
| (1.) | 3′-AA CTG CCA TAT AGA TCG ACG-5′ |
| (2.) | 3′-TG AAA CTG CCA TAT AGA TCG-5′ |
| (3.) | 3′-GGA ATG AAA CTG CCA TAT AG-3′ |

For the measurements with the Biacore device, a streptavidin coupled chip had to be prepared first. For this purpose, a CM 5 chip from Biacore was used which was appropriately modified. On its surface the CM 5 chip contains carboxyl group as functional groups which are activated with EDC/NHS and are subsequently reacted with a streptavidin derivative exhibiting an amino group. The biotinylated oligonucleotide is bound to the chip by the addition of 25 μl of a 50 μmolar solution. After attachment of the biotinylated model oligonucleotide, the corresponding complementary oligonucleotide (1^{st} to 3^{rd}) is bound by adding a 500 mM solution for 5 min (25 μl solution). The subsequent cleavage with a 10 mM silver nitrate solution takes place within 6 min by the addition of 30 μl solution. Rinsing then follows with a 0,5 molar EDTA solution. The subsequent detection shows complete cleavage; only the corresponding 10 mer, which may not be cleaved off, can be detected on the chip.

### Example 4

### Synthesis of Oligonucleotide Probes for Use on a Probe Array

For binding to a chip, the following oligonucleotides were synthesised:

5′-amino-AGC CCT TAC TTT GAC GGT ATA TCT-3′
5′-amino-AGC CCT TAC TTT GAC GGT ATA TCT-3′
(control sequence)

The 5′-ends of the oligonucleotides were modified with an amino link (Glen Research). The synthesis of the oligonucleotides is carried out as described in Example 2. A further modification at the 5′-end was carried out on the Applied Biosystem 394 synthesizer. A standard cycle whose coupling time was prolonged to 300 s was used for the synthesis. The purification and processing were again carried out as described in Example 2.

### Example 5

### Comparison of the Hybridisation Properties of an Oligonucleotide with a Bridged Phosphothioate Bond With an Unmodified Oligonucleotide.

Two oligonucleotides with the same sequence each 24 bases in length were immobilised on an epoxidised Pyrex glass surface. The synthesis and structure of the oligonucleotides have been described in Example 4. Both oligonucleotides were equipped with an amino modification at the 5′-end. One of the oligonucleotides was an unmodified DNA whilst the other oligonucleotide contained a backbone modification in approximately the centre of the molecule.

The backbone modification was generated by exchanging a phosphodiester bond of the oligonucleotide for a bridged phosphothioate bond. The bond thus formed differs from all other bonds of the oligonucleotide concerned. It can therefore be attacked and cleaved selectively, e.g. by heavy metal ions such as mercury ions and/or silver ions.

The deposition of the probes was carried out with an Eppendorf pipette (set at 0.μl) from a 10 μM solution of the oligonucleotides, in each case, in 0,5 M phosphate buffer at pH 8.0. Two drops of the phosphothioate oligonucleotide and one drop of the phosphate oligonucleotide were deposited per chip. The arrangement of the probes is illustrated in FIG. 3a.

The covalent linkage of the amino linker of the oligonucleotides to the epoxide surface of the array was achieved by allowing the deposited drops to dry at room temperature and then incubation for 30 min at 60° C. This was followed by washing of the chips according to the following protocol:
5 min in 100 ml of deionised H₂O+100 μl of triton±100
2×2 min in 100 ml of deionised H₂0 30 min in 100 ml of 100 mM KCl solution Rinsing for 1 min in 100 ml of deionised H₂O Drying

Hybridisation was then carried out with a completely complementary oligonucleotide, 24 bases in length, which was labelled at the 5′ end with a Cy3 dye (MWG Biotech, Ebersberg). For this purpose, 50 μl of a 100 nM solution of the complementary oligonucleotide in hybridisation buffer (0.25M NaPO₄, 4.5% SDS, 1 mM EDTA in 1×SSC) were added to a 1.5 ml reaction vessel (Eppendorf, Hamburg, Germany). After the addition of the chip, a denaturation step was carried out for 5 min at 95° C. This was followed by hybridisation for 1 h at 60° C. Each chip was washed with shaking in a thermo-shaker (Eppendorf, Hamburg, Germany) for 10 min at 30° C. in 2×SSC+0.2% SDS and 10 min at 30° C. in 2×SSC and subsequently for 10 min at 20° C. in 0.2×SSC (Maniatis et al., 1989). The volume was 500 μl in each case. The chip was dried for 5 min in a vacuum concentrator (Eppendorf, Hamburg, Germany). Hybridisation signals were detected under a Zeiss fluorescence microscope (Zeiss, Jena, Germany). The excitation took place in the incident light of a white light source using a set of filters suitable for Cy 3. The signals were recorded with a CCD camera (PCO-Sensicam, Kehlheim, Germany). The exposure time was 1000 ms.

A hybridisation signal with the same intensity was detected on both probe molecules (FIG. 3b). Consequently, both phosphothioate and phosphate oligonucleotides exhibit largely comparable hybridisation properties.

### Example 6

### Specific and Effective Cleavage of the Bridged Phosphothioate Bond in Immobilised Oligonucleotides With Silver Ions.

The experiment of Example 5 was repeated; however, the array surface was first treated with silver ions. As a result, the phosphothioate bonds were cleaved whereas the phosphodiester oligonucleotide bonds remained uncleaved. Following hybridisation, a strong hybridisation signal was detected on the non-modified oligonucleotide probes, whereas the signal on the phosphothioate-modified probes had largely disappeared. The principle of the experiment is shown in FIG. 4.

Two oligonucleotides with the same sequence, each 24 bases in length, were immobilised on an epoxidised Pyrex glass surface. The synthesis and structure of the oligonucleotides have been described in Example 4. Both oligonucleotides were equipped with an amino modification at the 5′-end. One of the oligonucleotides was unmodified DNA, whilst the other oligonucleotide had a backbone modification in approximately the centre of the molecule.

The backbone modification was produced by exchanging a phosphodiester bond of the oligonucleotide for a bridged phosphothioate bond. The bond thus formed differs from all other bonds of the oligonucleotide concerned. It can therefore be attacked and cleaved selectively, e.g. by heavy metal ions such as mercury ions or silver ions.

The deposition of the probes was carried out with an Eppendorf pipette (set at 0.μl) using a 10 μM solution of the oligonucleotides in each case, in 0.5 M phosphate buffer at pH 8.0. Two drops of the phosphothioate oligonucleotide and one drop of the phosphate oligonucleotide were deposited per chip. The arrangement of the probes is illustrated in FIG. 3.

The covalent linkage of the amino linker of the oligonucleotides to the epoxide surface of the array was achieved by allowing the deposited drops to dry at room temperature and subsequent incubation for 30 min at 60° C. This was followed by washing of the chips according to the following protocol:
5 min in 100 ml of deionised H₂O+100 μl of triton×100
2×2 min in 100 ml of deionised H₂O 30 min in 100 ml of 100 mM KCl solution Rinsing for 1 min in 100 ml of deionised H₂O Drying

For the selective cleavage of the phosphothioate bond, the chips were incubated for 20 min at 30° C. in 100 μl silver nitrate solution. The concentrations were 1 M, 200 mM, 50 mM and 10 mM. Following the silver nitrate cleavage, the chips were washed twice for 5 min in deionised water.

Subsequently, hybridisation was carried out with a completely complementary oligonucleotide of 24 bases in length which was labelled at the 5′-end with a Cy3 dye (MWG Biotech, Ebersberg). For this purpose, 50 μl of a 100 nM solution of the complementary oligonucleotide in hybridisation buffer (0.25M NaPO₄, 4.5% SDS, 1 mM EDTA in 1×SSC) were added to a 1.5 ml reaction vessel (Eppendorf, Hamburg, Germany). After addition of the chip, a denaturation step was carried out for 5 min at 95° C. This was followed by hybridisation for 1 h at 60° C. The chips were washed with shaking in a thermo-shaker (Eppendorf, Hamburg, Germany) for 10 min at 30° C. in 2×SSC+0.2% SDS and 10 min at 30° C. in 2×SSC and subsequently for 10 min at 20° C. in 0.2×SSC (Maniatis et al., 1989). The volume was 500 μl in each case. The chip was dried for 5 min in a vacuum concentrator (Eppendorf, Hamburg, Germany). The hybridisation signals were detected under a Zeiss fluorescence microscope (Zeiss, Jena, Germany). The excitation took place in the incident light of a white light source using a set of filters suitable for Cy 3. The signals were recorded with a CCD camera (PCO-Sensicam, Kehlheim, Germany). The exposure time was 1000 ms.

In comparison with hybridisation without silver nitrate treatment of the chip (Example 5), the signal intensity at the spots on which the phosphothioate oligo had been immobilised was substantially reduced and is near the limit of detection, whereas the signal intensity at the spot occupied by the phosphate oligo remained unchanged (compare FIG. 5). Consequently, the phosphothioate bond can be cleaved selectively and efficiently.

### Example 7

### Bond Cleavage in the Hybridised State and Examination of the Stability of the Cleaved Hybrid.

In this example, the same probe array was used as in Example 5. However, the hybridisation was first carried out with a complementary labelled oligonucleotide and a treatment with 50 mM AgNO₃ was only then carried out for a duration of 30 min at 0° C. Although bond cleavage of the phosphothioate oligonucleotide took place, it was possible to detect hybridisation both on the phosphothioate-modified oligonucleotide and on the unmodified phosphate oligonucleotide under suitable conditions, e.g. an increase in the ionic strength by the addition of 1M NaNO₃. After separation of the hybrid by melting and again hybridisation with the complementary 24 mer under stringent conditions, a strong hybridisation signal is detectable only on the uncleaved phosphate oligonucleotide. This shows that an efficient cleavage of the phosphothioate bond takes place also in the hybridised state, while the hybrid can be sufficiently stabilised under suitable conditions to prevent a disassociation after bond cleavage.

Two oligonucleotides with the same sequence, each 24 bases in length, were immobilised on an epoxidised Pyrex glass surface. The synthesis and structure of the oligonucleotides have been described in Example 4. Both oligonucleotides had an amino modification at the 5′-end. One of the oligonucleotides was unmodified DNA, whilst the other oligonucleotide had a backbone modification in approximately the centre of the molecule.

The backbone modification was produced by exchanging a phosphodiester bond of the oligonucleotide for a bridged phosphothioate bond. The bond thus formed differs from all other bonds of the oligonucleotide concerned. It can therefore be attacked and cleaved selectively, e.g. by heavy metal ions such as mercury ions or silver ions.

The deposition of the probes was carried out with an Eppendorf pipette (set at 0.1 μl) from a 10 μM solution of the oligonucleotides, in each case, in 0,5 M phosphate buffer at pH 8.0. Two drops of the phosphothioate oligonucleotide and one drop of the phosphate oligonucleotide were deposited per chip. The arrangement of the probes is illustrated in FIG. 3a.

The covalent linkage of the amino linker of the oligonucleotides to the epoxide surface of the array was achieved by allowing the deposited drops to dry at room temperature and subsequent incubation for 30 min at 60° C. This was followed by washing of the chips according to the following protocol:
5 min in 100 ml of deionised H₂O+100 μl of triton×100
2×2 min in 100 ml of deionised H₂O 30 min in 100 ml of 100 mM KCl solution Rinsing for 1 min in 100 ml of deionised H₂O Drying

Subsequently, hybridisation was carried out with a completely complementary oligonucleotide having 24 bases in length which was labelled at the 5′-end with a Cy3 dye (MWG Biotech, Ebersberg). For this purpose, 50 μl of a 100 nM solution of the complementary oligonucleotide in hybridisation buffer (0.25M NaPO₄, 4.5% SDS, 1 mM EDTA in 1×SSC) were added to a 1.5 ml reaction vessel (Eppendorf, Hamburg, Germany) . After addition of the chip, a denaturation step was carried out for 5 min at 95° C. This was followed by hybridisation for 1 h at 60° C. The chips were washed with shaking in a thermo-shaker (Eppendorf, Hamburg, Germany) for 10 min at 30° C. in 2×SSC+0.2% SDS and for 10 min at 30° C. in 2×SSC and subsequently for 10 min at 20° C. in 0.2×SSC (Maniatis et al., 1989) and treated for 5 min in a vacuum concentrator (Eppendorf, Hamburg, Germany). The hybridisation signals were detected under a Zeiss fluorescence microscope (Zeiss, Jena, Germany). The excitation took place in the incident light of a white light source and a set of filters suitable for Cy 3. The signals were recorded with a CCD camera (PCO-Sensicam, Kehlheim, Germany). The exposure time was 1000 ms.

For the selective cleavage of the phosphothioate bond, chips were incubated in 100 μl of 50 mM AgNO₃, 1 M NaNO₃ for 20 min at 0° C. After silver nitrate cleavage, the chips were washed 2×5 min in 500 μl of 1 M NaNO₃ on ice.

The chip was dried for 5 min in a vacuum concentrator (Eppendorf, Hamburg, Germany). The detection of the hybridisation signals was carried out as described above.

Subsequently, the chip was washed 3× for 10 min at 95° C. in 500 μl of deionised water. As a result, the hybrids were melted. A second hybridisation followed. 50 μl of a 100 nM solution of the completely complementary oligonucleotide, 24 bases in length, labelled at the 5′-end with a Cy3 dye (MWG Biotech, Ebersberg) in hybridisation buffer (0.25 M NaPO₄, 4.5% SDS, 1 mM EDTA in 1×SSC) were added to a 1.5 ml reaction vessel (Eppendorf, Hamburg, Germany). After addition of the chip, a denaturation step was carried out for 5 min at 95° C. The hybridisation was then carried out for 1 h at 60° C. The chips were washed with shaking in a thermo-shaker (Eppendorf, Hamburg, Germany) for 10 min at 30° C. in 2×SSC+0.2% SDS and for 10 min at 30° C. in 2×SSC and subsequently for 10 min at 20° C. in 0.2×SSC (Maniatis et al., 1989). The volume was 500 μl in each case. Subsequently, the hybridisation signals were detected as described above.

FIG. 6 shows, from left to the right, illustrations of the same array after different steps of the experiment: after hybridisation of the target, after bond cleavage as well as after separation of the hybrids by melting and again hybridisation under stringent conditions. On the one hand, it is shown that the bond cleavage is effective also in the hybridised state: after separation of the hybrids by melting and a further hybridisation, a strong hybridisation signal can be observed only of the non-cleavable phosphate oligonucleotide. At the phosphothioate oligonucleotide, only a slight hybridisation signal is observed at the limit of detection since the 10 mers remaining on the surface after the bond cleavage do not form stable hybrids under the stringent hybridisation conditions.

### Example 8

### Production and Quality Test of a Probe Array and Detection Using the Probe Array.

An array is prepared containing a total of 52 probes directed towards 8 in vitro RNAs. The probes are characterised by a cleavable bond and a label. Following the preparation of the array, the quality of the spots is checked. Subsequently, the hybridisation is carried out with a defined mixture of the 8 unlabelled in vitro RNAs. The labile bond is cut. After washing, the signals remaining on the array are detected. The signal intensities at the spots correspond to those of comparative experiments with labelled RNA and unlabelled, non-cleavable probes.

This documents the suitability of the method for detecting molecular interactions on probe arrays. In a series of tests of 10 experiments each carried out according to the conventional detection method and that of the invention, it was found that the scatter of the values determined by the method according to the invention is considerably lower than that obtained according to the conventional method.

a) Design and Production of the Probes

The design of the probes depends on the sequence of the in vitro RNAs to be detected. The design process considers the accessibility of the target region for a potential probe, possible interactions with parts of the same molecule, but also possible interactions between target molecules in solution, or undesirable interactions, which negatively influence the specificity, with different probes. It is considered that a cleavage site ought to be inserted. This cleavage site is the respective T, which is arranged nearest to the centre of the probe. It has a 5′-phosphothioate bond. Apart from the 52 target-specific probes (1-52), 3 control probes (probe 53-55) are produced. Probes 53 and 54 possess no cleavable bond. Probe 53 carries a label, whereas probe 54 is unlabelled. Probe 55 does not hybridise with the target sequences and is used to define the experimental background signal.

After determining the probe sequence, these are produced synthetically as illustrated e.g. in Example 2. The sequences of the probes are indicated in the following Table.

| | | | |
|---|---|---|---|
| 1 | TCTAAAACCTGGCCAGCAATCATTC | 3′Cy3 | 5′NH2 |
| | | | phospho- |
| | | | thioate |
| 2 | GCCCGGGCATTTCTCTCATTAACAT | 3′Cy3 | 5′NH2 |
| | | | phospho- |
| | | | thioate |
| 3 | TTCGAAAAGATTGCCTCCACATCAG | 3′Cy3 | 5′NH2 |
| | | | phospho- |
| | | | thioate |
| 4 | GTCTCATCTTTCTTCACGGAGCTGC | 3′Cy3 | 5′NH2 |
| | | | phospho- |
| | | | thioate |
| 5 | TGCTTGTTTGCTCTGTTCCTTTTCA | 3′Cy3 | 5′NH2 |
| | | | phospho- |
| | | | thioate |
| 6 | TCCAGGTTTTCCAGGAGAGAATCCA | 3′Cy3 | 5′NH2 |
| | | | phospho- |
| | | | thioate |
| 7 | TCTGGGTCAGCTCCTTCTTAATGGC | 3′Cy3 | 5′NH2 |
| | | | phospho- |
| | | | thioate |
| 8 | TCTAGAGGATGCATTTGACATGCCA | 3′Cy3 | 5′NH2 |
| | | | phospho- |
| | | | thioate |
| 9 | TGTTACATTTGTGTTGAACTGCCCC | 3′Cy3 | 5′NH2 |
| | | | phospho- |
| | | | thioate |
| 10 | AATGAGATTGCCTTTGCAGTTAGGG | 3′Cy3 | 5′NH2 |
| | | | phospho- |
| | | | thioate |
| 11 | TTCTTTTGCCCTAGCTCCAAGTTCA | 3′Cy3 | 5′NH2 |
| | | | phospho- |
| | | | thioate |
| 12 | TCGTCCAACAAATACTTTGCGATCA | 3′Cy3 | 5′NH2 |
| | | | phospho- |
| | | | thioate |
| 13 | AATAGCTCTTTCAGCTGCTTCCTGC | 3′Cy3 | 5′NH2 |
| | | | phospho- |
| | | | thioate |
| 14 | TACAAATCCATAGCCCTTGGAACCA | 3′Cy3 | 5′NH2 |
| | | | phospho- |
| | | | thioate |
| 15 | TATGTTGCCTACTCCACTTTTGCGA | 3′Cy3 | 5′NH2 |
| | | | phospho- |
| | | | thioate |
| 16 | TGTTCAAATTTGCGCTTAAGTTCCG | 3′Cy3 | 5′NH2 |
| | | | phospho- |
| | | | thioate |
| 17 | TTTGTTTTCCATTGAGCTCCTTTCC | 3′Cy3 | 5′NH2 |
| | | | phospho- |
| | | | thioate |
| 18 | TTACTTTCACACTTAAGGCAGGCCC | 3′Cy3 | 5′NH2 |
| | | | phospho- |
| | | | thioate |
| 19 | GACATGACTCGTGGAACCTGTGAAG | 3′Cy3 | 5′NH2 |
| | | | phospho- |
| | | | thioate |
| 20 | TAAATGGTGGTCTAGGAGCAGCTGG | 3′Cy3 | 5′NH2 |
| | | | phospho- |
| | | | thioate |
| 21 | TTGGCTAGGAGGATAGTATGCAGCA | 3′Cy3 | 5′NH2 |
| | | | phospho- |
| | | | thioate |
| 22 | AACACAGCGTGTTGCTAACACATCA | 3′Cy3 | 5′NH2 |
| | | | phospho- |
| | | | thioate |
| 23 | CTGTCCGCACCGTTCCACAGTATAA | 3′Cy3 | 5′NH2 |
| | | | phospho- |
| | | | thioate |
| 24 | CAGCAACATCTTAATGCACAGCCAC | 3′Cy3 | 5′NH2 |
| | | | phospho- |
| | | | thioate |
| 25 | AAGTTACAATGCAACAGCCTGCTGT | 3′Cy3 | 5′NH2 |
| | | | phospho- |
| | | | thioate |
| 26 | TCTAAAACCTGGCCAGCAATCATTCTGCCA | 3′Cy3 | 5′NH2 |
| | | | phospho- |
| | | | thioate |
| 27 | CTCTCCTGCTACAGCAGCCCGGGCATTTCT | 3′Cy3 | 5′NH2 |
| | | | phospho- |
| | | | thioate |
| 28 | CGAAGGCAAAGCCCTTATGAACAGAGCAGC | 3′Cy3 | 5′NH2 |
| | | | phospho- |
| | | | thioate |
| 29 | TCCCAATGAATACACGGGAGTTCATGGAGC | 3′Cy3 | 5′NH2 |
| | | | phospho- |
| | | | thioate |
| 30 | GGATCTGTCTTGTTGGTAACGTTGCTGGCC | 3′Cy3 | 5′NH2 |
| | | | phospho- |
| | | | thioate |
| 31 | TCATCTTTCTTCACGGAGCTGCTGCTCTGC | 3′Cy3 | 5′NH2 |
| | | | phospho- |
| | | | thioate |
| 32 | TGGGTCAGCTCCTTCTTAATGGCCTGAAGG | 3′Cy3 | 5′NH2 |
| | | | phospho- |
| | | | thioate |
| 33 | AGAATTGAAGCCACTTTTGCCCCTTCGTGA | 3′Cy3 | 5′NH2 |
| | | | phospho- |
| | | | thioate |
| 34 | TACATTTGTGTTGAACTGCCCCACACAGCA | 3′Cy3 | 5′NH2 |
| | | | phospho- |
| | | | thioate |
| 35 | TCAAAGGAAGTGAAAATGGGACTAGGCGCG | 3′Cy3 | 5′NH2 |
| | | | Phospho- |
| | | | thioate |
| 36 | ATGTGCTTAAGAGTCATCCTCGCCATTGGC | 3′Cy3 | 5′NH2 |
| | | | phospho- |
| | | | thioate |
| 37 | AGCTCTTTCAGCTGCTTCCTGCGTCTCAAA | 3′Cy3 | 5′NH2 |
| | | | phospho- |
| | | | thioate |
| 38 | ACTCCACTTTTGCGAAGTGATGGATCACGC | 3′Cy3 | 5′NH2 |
| | | | phospho- |
| | | | thioate |
| 39 | GAGACCACATGATGCGTACTGGCTTGCCCT | 3′Cy3 | 5′NH2 |
| | | | phospho- |
| | | | thioate |
| 40 | TCAAAATTCATGGTGTCCAAAGCACGCTCC | 3′Cy3 | 5′NH2 |
| | | | phospho- |
| | | | thioate |
| 41 | GCCGGCTGCTGGAAGTTCACATACGCGTAG | 3′Cy3 | 5′NH2 |
| | | | phospho- |
| | | | thioate |
| 42 | TTCAAATTTGCGCTTAAGTTCCGTCTGCCG | 3′Cy3 | 5′NH2 |
| | | | phospho- |
| | | | thioate |
| 43 | TTGAGCTCCTTTCCGTTCATCTCATCCACA | 3′Cy3 | 5′NH2 |
| | | | phospho- |
| | | | thioate |
| 44 | AAGGCGCTCATCATCCATGTCTTCTCCAAA | 3′Cy3 | 5′NH2 |
| | | | phospho- |
| | | | thioate |
| 45 | CATGACTCGTGGAACCTGTGAAGAAGCTGG | 3′Cy3 | 5′NH2 |
| | | | phospho- |
| | | | thioate |
| 46 | ACTAAATGGTGGTCTAGGAGCAGCTGGGCG | 3′Cy3 | 5′NH2 |
| | | | phospho- |
| | | | thioate |
| 47 | AGCACCGGGCATATTTTGGAATGGATGAGG | 3′Cy3 | 5′NH2 |
| | | | phospho- |
| | | | thioate |
| 48 | ACCCTGAGCAGTCCAGCGAGGACTTGGTCT | 3′Cy3 | 5′NH2 |
| | | | phospho- |
| | | | thioate |
| 49 | CTACTCCTGCTGTCCGCACCGTTCCACAGT | 3′Cy3 | 5′NH2 |
| | | | phospho- |
| | | | thioate |
| 50 | TGCAGGAGTTCGCAATCCTCAGCAACATCT | 3′Cy3 | 5′NH2 |
| | | | phospho- |
| | | | thioate |
| 51 | TGCACAGCCACAAGTTACAATGCAACAGCC | 3′Cy3 | 5′NH2 |
| | | | phospho- |
| | | | thioate |
| 52 | TCAGGAACCTTTGACTGCTTCCATGTTGGC | 3′Cy3 | 5′NH2 |
| | | | phospho- |
| | | | thioate |
| 53 | CCTCTGCAGACTACTATTAC | 3′Cy3 | 5′NH2 |
| 54 | CCTCTGCAGACTACTATTAC | | 5′NH2 |
| 55 | CCTCTGCAGACTACTATTAC | 3′Cy3 | 5′NH2 |
| | | | phospho- |
| | | | thioate |

b) Production of the Array

The arrays are produced by spotting of specific probes on preproduced arraying substrates. Epoxidised glass supports are used as substrates. The target-specific probes listed above not only have the phosphothioate modification, but also have Cy3 labelling at the 3′-end of the sequence and an amino link at the 5′-terminus. In addition to the target-specific probes, probe 55 is hybridised as background control and different mixture ratios of probes 53 and 54 in the range of 1 to 1: 10,000 are immobilised to establish a calibration series for the calibration of the results. The probes are present in microtiter plates in a concentration of 10 μM in 0.5 M phosphate buffer. Spotting of the probes is carried out with a spotting system from Biorobotics (Microgrid II). Following the deposition of the probes on the surface, the arrays are baked for 30 min at 60° C. and washed according to the following protocol:
5 min in 600 ml of deionised H₂O+600 μl of triton×100
2×2 min in 600 ml of deionised H₂O 30 min in 600 ml of 100 mM KCl solution Rinsing for 1 min in 600 ml of deionised H₂O Drying

The probes are arranged in 3 subarrays each in triple redundancy.

c) Quality Control

The quality of the immobilised probes is controlled by detecting the fluorescent signal on the array. The signal intensities Sₒ are measured with a laser scanner of the Scan array 4000 type (GSI-Lumonics, USA). The intensities are standardised to a value by providing the individual intensities with a correction factor (kₐ) . From this, the number of molecules per spot in relation to other spots can be derived as a direct function. The factors are saved and used to determine whether the array as a whole can be used for the intended analyses. The measured intensities/spot are standardized to a value of 1 for each spot (Sₒ./k_{a n}=1).

d) Hybridisation Targets

Eight in vitro RNAs with the following sequence segments are used:

RNA 1:
5′UCUAGAAAUAAUUUUGUUUAACUUUAAGAAGGAGAUAUACAUAUGAACCCCAGUGCCCCC
AGCUACCCCAUGGCCUCGCUCUACGUGGGGGACCUCCACCCCGACGUGACCGAGGCGAUGCU
CUACGAGAAGUUCAGCCCGGCCGGGCCCAUCCUCUCCAUCCGGGUCUGCAGGGACAUGAUCA
CCCGCCGCUCCUUGGGCUACGCGUAUGUGAACUUCCAGCAGCCGGCGGACGCGGAGCGUGCU
UUGGACACCAUGAAUUUUGAUGUUAUAAAGGGCAAGCCAGUACGCAUCAUGUGGUCUCAGCG
UGAUCCAUCACUUCGCAAAAGUGGAGUAGGCAACAUAUUCAUUAAAAAUCUGGACAAAUCCA
UUGAUAAUAAAGCACUGUAUGAUACAUUUUCUGCUUUUGGUAACAUCCUUUCAUGUAAGGUG
GUUUGUGAUGAAAAUGGUUCCAAGGGCUAUGGAUUUGUACACUUUGAGACGCAGGAAGCAGC
UGAAAGAGCUAUUGAAAAAAUGAAUGGAAUGCUCCUAAAUGAUCGCAAAGUAUUUGUUGGAC
GAUUUAAGUCUCGUAAAGAACGAGAAGCUGAACUUGGAGCUAGGGCAAAAGAAUUC3′
RNA 2:
5′AACUGCUUUCUGGGCAGCCUCUUUAGCUUGGUGGGCUUGUAGUACAGCUACAGCUUCAUC
AACCUUAGAACGGAGUGACUCUGGAGACUCGAGCAUAUGAAGAAGUUCUGAAUUAUCAAUCU
CCAACAACAUGCCAGUGAUUUUACCAGCAAGAGUAGGGUGCAUGGCUUGAAUAAGAGGAAAC
AGCCGUUCACCCAACAUUUGCUUUUGCUCUUGAGGAGGGGCAGAUGCCAACAUGGAAGCAGU
CAAAGGUUCCUGACCUUGUACAUGAACAGCAGGCUGUUGCAUUGUAACUUGUGGCUGUGCAU
UAAGAUGUUGCUGAGGAUUGCGAACUCCUGCAGCAUAUUUAUACUGUGGAACGGUGCGGACA
GCAGGAGUAGCUGCAGCGGCUGCAGCUGCAGGACGUGGACCCAUUGUCUGUGUUGAUGUGUU
AGCAACACGCUGUGUUG3′
RNA 3:
5′UCUAGAAAAAUAAUUAGUGUUAUAGUCUUAAGAUUUGUUUUCUAAAGUUG
AUACUGUGGGUUAUUUUUGUGAACAGCCUGAUGUUUGGGACCUUUUUUCCUC
AAAAUAAACAAGUCCUUAUUAAACCAGGAAUUUGGAGAAAAAAAAAAGGAAUUC3′
RNA 4:
5′GAAUUCCAAACCCGGGAGUAGGAGACUCAGAAUCGAAUCUCUUCUCCCUCCCCUUCUUGU
GAGAUUUUUUUGAUCUUCAGCUACAUUUUCGGCUUUGUGAGAAACCUUACCAUCAAACACGA
UGGCCAGCAACGUUACCAACAAGACAGAUCCUCGCUCCAUGAACUCCCGUGUAUUCAUUGGG
AAUCUCAACACUCUUGUGGUCAAGAAAUCUGAUGUGGAGGCAAUCUUUUCGAAGUAUGGCAA
AAUUGUGGGCUGCUCUGUUCAUAAGGGCUUUGCCUUCGUUCAGUAUGUUAAUGAGAGAAAUG
CCCGGGCUGCUGUAGCAGGAGAGGAUGGCAGAAUGAUUGCUGGCCAGGUUUUAGAUAUUAAC
CUGGCUGCAG3′
RNA 5:
5′GAAUUCACCAAUGUUUACAUCAAGAAUUUUGGAGAAGACAUGGAUGAUGAGCGCCUUAAG
GAUCUCUUUGGCAAGUUUGGGCCUGCCUUAAGUGUGAAAGUAAUGACUGAUGAAAGUGGAAA
AUCCAAAGGAUUUGGAUUUGUAAGCUUUGAAAGGCAUGAAGAUGCACAGAAAGCUGUGGAUG
AGAUGAACGGAAAGGAGCUCAAUGGAAAACAAAUUUAUGUUGGUCGAGCUCAGAAAAAGGUG
GAACGGCAGACGGAACUUAAGCGCAAAUUUGAACAGAUGAAACAAGAUAGGAUCACCAGAUA
CCAGGGUGUUAAUCUUUAUGUGAAAAAUCUUGAUGAUGGUAUUGAUGAUGAACGUCUCCGGA
AAGAGUUUUCUCCAUUUGGUACAAUCACUAG3′
RNA 6:
5′AGUGCAAAGGUUAUGAUGGAGGGUGGUCGCAGCAAAGGGUUUGGUUUUGUAUGUUUCUCC
UCCCCAGAAGAAGCCACUAAAGCAGUUACAGAAAUGAACGGUAGAAUUGUGGCCACAAAGCC
AUUGUAUGUAGCUUUAGCUCAGCGCAAAGAAGAGCGCCAGGCUCACCUCACUAACCAGUAUA
UGCAGAGAAUGGCAAGUGUACGAGCUGUUCCCAACCCUGUAAUCAACCCCUACCAGCCAGCA
CCUCCUUCAGGUUACUUCAUGGCAGCUAUCCCACAGACUCAGAACCGUGCUGCAUACUAUCC
UCCUAGCCAAAUUGCUCAACUAAGACCAAGUCCUCGCUGGACUGCUCAGGGUGCCAGACCUC
AUCCAUUCCAAAAUAUGCCCGGUGCUAUCCGCCCAGCUGCUCCUAGACCACCAUUUAGUACU
AUGAGACCAGCUUCUUCACAGGUUCCACGAGUCAUGUC3′
RNA 7:
5′CUGCAGCGGAGAUGUACGGCUCCUCUUUUGACUUGGACUAUGACUUUCAACGGGACUAUU
AUGAUAGGAUGUACAGUUACCCAGCACGUGUACCUCCUCCUCCUCCUAUUGCUCGGGCUGUA
GUGCCCUCGAAACGUCAGCGUGUAUCAGGAAACACUUCACGAAGGGGCAAAAGUGGCUUCAA
UUCUAAGAGUGGACAGCGGGGAUCUUCCAAGUCUGGAAAGUUGAAAGGAGAUGACCUUCAGG
CCAUUAAGAAGGAGCUGACCCAGAUAAAACAAAAAGUGGAUUCUCUCCUGGAAAACCUGGAA
AAAAUUGAAAAGGAACAGAGCAAACAAGCAGUAGAGAUGAAGAAUGAUAAGUCAGAAGAGGA
GCAGAGCAGCAGCUCCGUGAAGAAAGAUGAGACUAAUGUGAAGAUGGAGUCUGAGGGGGGUG
CAGAUGACUCUGCUGAGGAGGGGGACCUACUGGAUGAUGAUGAUAAUGAAGAUCGGGGGGAU
GACCAGCUG3′
RNA 8:
5′cagcuggaguugaucaaggaugaugaaaaagaggcugaggaaggagaggaugacagagac
agcgccaauggcgaggaugacucuuaagcacauagugggguuuagaaaucuuaucccauuau
uucuuuaccuaggcgcuugucuaagaucaaauuuuucaccagauccucuccccuaguaucuu
cagcacaugcucacuguucuccccauccuuguccuucccauguucauuaauucauauugccc
cgcgccuagucccauuuucacuuccuuugacgcuccuaguaguuuuguuaagucuuacccug
aaauuuuugcuuuuaauuuugauaccucuuuaugacuuaacaauaaaaaggauguaugguuu
uuaucaacugucuccaaaauaaucucuuguuaugcagggaguacaguucuuuucauucauac
auaaguucaguaguugcuucccuaacugcaaaggcaaucucauuuaguugaguagcucuuga
aagcagcuuugaguuagaaguauguguguuacacccucacauuagugugcuguguggggcag
uucaacacaaauguaacaauuauuuuugugaaugagaguuggcaugucaaaugcauccucua
ga3′

e) Hybridisation of RNA

The RNAs produced in vitro are purified from a 5% denaturing PAA gel (Maniatis et al., 1989), precipitated and taken up in deionised water, measured spectrophotometrically and adjusted to a uniform concentration of 2 μM. The RNAs are taken up in an equimolecular ratio in 100 μl of hybridisation buffer (0.25 M NaPO₄, 4.5% SDS, 1 mM EDTA in 1×SSC). The final concentration is 40 nM. The hybridisation solution is denatured for 5 minutes at 80° C. The surface of the slide occupied with DNA is covered with a hybridisation chamber (Hybriwell, Sigma, Deisenhofen, Germany). The slide is preheated to 50° C. on a thermo-shaker equipped with a microtiter plate top unit (Eppendorf, Hamburg, Germany). The denatured hybridisation solution is then added and the hybridisation chamber is closed according to the manufacturer's instructions. Incubation is continued for 60 min at 50° C. Subsequently, the hybridisation solution is discarded, the hybridisation chamber is removed and the slides are washed by shaking for 10 min at 30° C. in 2×SSC+0.2% SDS, and for 10 min at 20° C. in 2×SSC and for 10 min at 20° C. in 0.2×SSC each and dried with compressed air.

f) Selective Cleavage of the Phosphothioate Bond

For the selective cleavage of the phosphothioate bond, the arrays are incubated by shaking in a plastic vessel in 2 ml of 50 mM AgNO₃, 1 M NaNO₃, for 20 min at 0° C. such that the arrays are completely overlaid. Following the silver nitrate cleavage, the chips are washed 2×5 min in 50 ml of 1 M NaNO₃ on ice and subsequently dried under compressed air or argon. Care should be taken to ensure that the solution is completely removed since, otherwise, drying marks are formed. The hybridisation signals S₁ are detected with a laser scanner of the Scan array 4000 type (GSI-Lumonics, USA). The image is produced with the identical detector settings to those used for the quality control. Subsequently, the array is corrected to the probe-specific intensities by using the factors obtained in quality control. The equation (S_{1 n}*k_{a n}=S_{2 n}.) is applied for each spot.

Subsequently, the background value is subtracted from all the values. This represents the average of the signal intensities S_{2 n} of all spots which are occupied by oligonucleotide 55.

S_{2 n}′=S_{2 n}−S_{2 Oligo55} is obtained.

The corrected values give a considerably more realistic picture of the intensity distribution and thus the distribution of the concentration of the targets in solution. This is of significance in particular for quantitative measurements of the differential gene expression but also for assessing the validity of signals. The normalised signal remaining on the spots is directly proportional to the quantity of the targets present in solution.

When comparing the intensities of different arrays, additional scaling is carried out. A factor is calculated which represents the differences in the signal intensities of the calibration spots in which oligo 53 and oligo 54 have been mixed in different molar ratios. Only those calibration spots are taken into consideration when determining the scaling factor which are within the dynamic range of detection.

The S_{2 n}′ values determined on each array are multiplied by the scaling factor.

g) Evaluation of the Results

The evaluation of the results is carried out using the Iconoclust software package (Clondiag) which allows both the determination of the above-mentioned factors and their calculation by means of program scripts, when the above-mentioned arrays are used. In principle, this is also possible by using a spread sheet.

### Example 9

### Synthesis of Oligonucleotides With a Bridged Phosphothioate Bond on Array Surfaces.

Two oligonucleotides with the same sequence 24 bases in length respectively, among others, were synthesised on an array surface. One of the oligonucleotides was an unmodified oligonucleotide produced by synthesis with standard phosphoramidites. A phosphorus-sulfur bond was introduced into the other oligonucleotide of identical sequence by coupling of a phosphothioate amidite. This is different from all the other bonds of the oligonucleotide concerned. It can therefore be selectively attacked and cleaved e.g. by heavy metal ions such as mercury ions or silver ions. This example shows the possibility of synthesising oligonucleotides with a bridged phosphothioate bond on array surfaces.

Array Production

On a four inch Borofloat wafer (PEG surface) modified with hydroxyl groups, the sequence (3′→5′) TCT-ATA-TGG-CAG was synthesised on an OligoPilotII (Pharmacia) by the standard phosphoramidite method while retaining the last DMT protective group. This was then removed at defined positions by deprotecting by means of a 128 μm mask (4 channels per chip). Subsequently, a dT amidite (DMT-ON) was coupled to the sites which had then become accessible for synthesis. Subsequently, the same 128 μm mask was used for again deprotection; however, its position was shifted by 256 μm in comparison with the first mask deprotection.

Before further use, the wafer was sawn into discs of a size of 3.4×3.4 mm. The following synthesis steps were carried out on an Expedite (Applied Biosystems):

To couple the cleavable 5′-(S-dimethoxytrityl)-mercapto-5′-deoxythymidine-3′-phosphoramidite (0,1 M solution), standard coupling protocols of the 1 μmol scale were modified: coupling time: 900 s, deblocking: 250 s, rinsing: 600 s with a 220 mM DTT solution in THF/pyridine/water (7/1/2). Subsequently, a dT-amidite was coupled, followed by the remaining sequence (3′→5′) CAT-TCC-CGA (deblocking, capping and oxidation corresponding to the standard protocol, 0.2 μmole scale). A solution with a lower iodine concentration (0.02 M iodine, Roth) was used for the oxidation step. To remove the base protective groups, the arrays were subsequently treated in 30-33% ammonia (Roth) for 35 min at 55° C.

Hybridisation

Subsequently, hybridisation was carried out with a completely complementary oligonucleotide 24 bases in length which was labelled at the 5′-end with a Cy3 dye (MWG Biotech, Ebersberg). For this purpose, 50 μl of a 10 nM solution of the complementary oligonucleotide in hybridisation buffer (6×SSPE, 0.1% SDS) were added to a 1.5 ml reaction vessel (Eppendorf, Hamburg, Germany). After the addition of the chip, a denaturation step was carried out for 5 min at 95° C. This was followed by hybridisation for 1 h at 50° C. The chips were washed with shaking in a thermo-shaker (Eppendorf, Hamburg, Germany) for 10 min at 30° C. in 2×SSC+0.2% SDS and for 10 min at 30° C. in 2×SSC and subsequently for 10 min at 20° C. in 0.2×SSC (Maniatis et al., 1989). The volume was 500 μl in each case. The chip was dried for 5 min in a vacuum concentrator (Eppendorf, Hamburg, Germany). The hybridisation signals were detected with a Zeiss fluorescence microscope (Zeiss, Jena, Germany). The excitation took place in the incident light of a white light source using a set of filters suitable for Cy3. The signals were recorded with a CCD camera (PCO-Sensicam, Kehlheim, Germany). The exposure time was 1000 ms.

The image of the array following hybridisation is shown in FIG. 8. P identifies the tracks or lanes containing the unmodified oligonucleotide (match probe). The tracks characterised by PT contain the oligonucleotide with the same sequence (match probe) with the phosphothioate modification. The tracks in between contain an oligonucleotide which differs from the two others by a T deletion approximately in the centre of the molecule (mismatch probe). The strong hybridisation signals in the region of the phosphothioate-modified oligonucleotide show that the incorporation of the 5′-(S-dimethoxytrityl)-mercapto-5′-deoxythymidine-3′-phosphoramidite on array surfaces is possible, probes with good hybridisation properties being formed.

### Example 10

Synthesis of oligonucleotides with a bridged phosphothioate bond on array surfaces and detection of the cleavability of the bond by subsequent hybridisation.

In this experiment, an array was produced as in Example 9 and subsequently subjected to conditions causing a cleavage of the phosphothioate bond. The cleavage of the bond was subsequently detected by hybridisation with a complementary oligonucleotide.

Cleavage of the phosphothioate bond with silver ions

The arrays were treated in a reaction vessel (Eppendorf) with a 50 mM silver nitrate solution for 15 min at 30° C. Subsequently, rinsing was carried out in the following sequence: 1× aqua dest. 50° C. 10 min, 2× aqua dest. 22° C. 10 min each. The arrays were dried in a vacuum concentrator (Eppendorf).

Hybridisation

Subsequently, hybridisation was carried out with a completely complementary oligonucleotide, 24 bases in length, which was labelled at the 5′-end with a Cy3 dye (MWG Biotech, Ebersberg). For this purpose, 50 μl of a 10 nM solution of the complementary oligonucleotide in 6×SSPE, 0.1% SDS (Maniatis et al., 1989) were added to a 1.5 ml reaction vessel (Eppendorf, Hamburg, Germany). After the addition of the chip, a denaturation step was carried out for 5 min at 95° C. This was followed by hybridisation for 1 h at 50° C. The chips were washed with shaking in a thermo-shaker (Eppendorf, Hamburg, Germany) for 10 min respectively at 30° C. in 2×SSC+0.2% SDS and 2×SSC and subsequently for 10 min at 20° C. in 0.2×SSC (Maniatis et al., 1989). The volume was 500 μl in each case. The chip was dried for 5 min in a vacuum concentrator (Eppendorf, Hamburg, Germany). The hybridisation signals were detected with a Zeiss fluorescence microscope (Zeiss, Jena, Germany). The excitation took place in the incident light of a white light source using a set of filters suitable for Cyanin 3. The signals were recorded with a CCD camera (PCO-Sensicam, Kehlheim, Germany). The exposure time was 1000 ms.

The images of the array after cleavage of the phosphothioate bond and subsequent hybridisation are shown in FIG. 9. P identifies the tracks containing the unmodified oligonucleotide (match probe). The tracks characterised by PT contain the oligonucleotide of identical sequence (match probe) with the phosphothioate modification. The tracks in between contain an oligonucleotide which differs from the two others by a T deletion approximately in the centre of the molecule (mismatch probe).

The hybridisation signals in the region of the phosphothioate-modified oligonucleotide (PT), which are substantially reduced compared to those on the unmodified oligonucleotide (P), show that the oligonucleotides with a phosphothioate bond produced on the array surfaces can be efficiently cut by silver ions.

### Example 11

Synthesis of labelled oligonucleotides with a bridged phosphothioate bond on array surfaces, hybridisation with a complementary unlabelled oligonucleotide and detection of hybridisation by cleavage of the phosphothioate bond.

Two oligonucleotides with the same sequence each 24 bases in length were synthesised, among other things, on an array surface. One of the oligonucleotides was an unmodified oligonucleotide produced by synthesis with standard phosphoramidites. A phosphorus-sulfur bond was introduced into the other oligonucleotide of identical sequence by coupling of a phosphothioate amidite. This is different from all the other bonds of the oligonucleotide concerned. It can therefore be selectively attacked and cleaved, e.g. by heavy metal ions such as mercury ions and/or silver ions. The probes synthesised on the array were labelled at their 5′-end with a Cy3 dye.

Subsequently, the phosphothioate bond of oligonucleotide probes was cut on two identical arrays. One of the arrays was first hybridised with an unlabelled oligonucleotide complementary to the probe. It was possible to detect the hybridisation by retaining the labelling of the probe on the array as a result of the hybridisation whereas it was lost in the case of the non-hybridised array.

Array Production

The array was initially produced according to the same protocol as was used in the two previous practical Examples 9 and 10. Subsequently, coupling of the Cyanine 3 dye (Amersham Pharmacia) was carried out on DMT-off without capping by hand according to the following protocol:

Coupling of a mixture of Cy3 amidite and dA amidite (0.1 M stock solutions mixed in each case in a ratio of 1:9) was carried out under an argon atmosphere. First, the chips were placed into small columns and rinsed with anhydrous acetonitrile. Subsequently, a 2 ml mixture of 1 ml Cy3 amidite/dA amidite and 1 ml DCI activator was passed over the column within 5 minutes. Subsequently, rinsing was carried out with 10 ml of acetonitrile, then oxidated with 1 ml oxidiser and finally rinsing again with 10 ml of acetonitrile.

Hybridisation

One of the two arrays was hybridised with the complementary oligonucleotide as described in Examples 9 and 10.

Selective cleavage of the phosphothioate bond For the selective cleavage of the phosphothioate bond, both the hybridised and the non-hybridised array were incubated for 20 min at 0° C. with shaking each in a reaction vessel (Eppendorf) in 100 μl of 50 mM AgNO₃, 1 M NaNO₃ respectively such that the arrays were completely covered. Following silver nitrate cleavage, the chips were washed 2×5 min in 50 ml of 1M NaNO₃ on ice and subsequently dried in a vacuum concentrator (Eppendorf, Hamburg, Germany).

The hybridisation signals were detected with a laser scanner of the Scan array

4000 type (GSI-Lumonics, USA).

Results

In the case of the non-hybridised array, a distinct weakening of the signal in the tracks containing the phosphothioate probes occurred following silver cleavage whereas the signal remained unchanged in all other tracks.

In contrast, the signal in the phosphothioate tracks was largely retained in the array treated with silver ions only after hybridisation. This result shows that oligonucleotide probes synthesised in situ with a bridged phosphothioate bond can be used for the detection of hybrids with unlabelled targets.

### Example 12

### Preparation of 5′-O-(4,4′-dimethoxytrityl)-thymidylyl-(3′→5′)-3′-O-[(2-cyanoethyl)-N,N-diisopropylamidophosphoramidite]-2′-deoxythymidine

The synthesis of 5′-O-(4,4′-dimethoxytrityl)-thymidylyl-(3′→5′)-3′-O-[(2-cyanoethyl)-N,N-diisopropylamidophosphoramidite]-2′-deoxythymidine is accomplished according to Example 12 using the synthesis scheme shown in FIG. 10.

### 5′-S-[9- (4-methoxyphenyl)xanthene-9-thio]-3′-O-benzoyl-2′-deoxythymidine (7)

Size of batch:

1.47 g of 5′-S-[9-(4-methoxyphenyl)xanthene-9-thio]-2′-deoxythymidine (5) (2.7 mmole) 0.568 g of benzoyl chloride (4.04 mmole) (For an illustration compare Example 13) 20 ml of pyridine

Experimental Execution:

5′-S-[9-(4-methoxyphenyl)xanthene-9-thio]-2′-deoxythymidine (5) is dissolved in pyridine under a protective gas atmosphere and benzoyl chloride is added slowly dropwise with cooling with ice. After 3 h, no further starting product can be detected by DC (solvent DCM:MeOH 9:1). The batch is poured into 400 ml of iced water and the precipitate formed is filtered with suction. Since the precipitate becomes oily on the frit, the oily solid is dissolved in dichloromethane and the organic phase is extracted with saturated sodium chloride solution. The organic phase is dried on magnesium sulfate and treated in the rotary evaporator.

| R = | 1.66 g | |
|---|---|---|
| Analysis: | | |
| Yield | 93% | |
| Rf value: | solvent dichloromethane:methanol 95:5 | 0.52 |
| | dichloromethane:methanol 9:1 | 0.77 |

| | |
|---|---|
| ESI(−)MS | C₃₇H₃₂N₂O₇S |
| Calculated | 648.65 |
| Found | 671.3 (Na salt) |
| NMR: | ¹H-NMR(250 MHz, CDCl₃): δ = 8.87(br. s, 1H, N-H), |
| | 8.58-8.55(m, 1H, H-6), 7.90-7.87(m, |
| | 2H, ortho-H benzoyl), 7.52-7.35(m, 15 H, aromatic), |
| | 6.18-6.12(dd, 1H, H-1′), 5.21-4.95(m, 1H, H-3′), |
| | 3.95-3.94(m, 1H, H-4′), 3.72(s, 3H, OCH₃), 2.59-2.54(m, |
| | 2H, H-5′, H-5′), 2.39-2.3(m, 1H, H-2′), 2.11-2.05(m, 1H, |
| | H-2″), 1.88(d 3H, CH₃-T) |

5′-thiol-3′-O-benzoyl-2′-deoxythymidine (8)

Size of batch:

| | |
|---|---|
| 1.5 g of 5′-S-[9-(4-methoxyphenyl)xanthene-9- | (2.31 mmole) |
| thio]-3′-OBzl-2′-deoxythymidine (7) | |
| 600 mg of silver nitrate | (3.53 mmole) |
| 300 μl of pyridine | |
| 80 ml of methanol | |
| 0.5 ml of acetic acid | |
| 40 ml of methanol | |

Experimental Execution:

5′-S-[9- (4-methoxyphenyl)xanthene-9-thio]-3′-OBzl-2′-deoxythymidine (7) is dissolved in methanol under a protective gas atmosphere. Then follows the addition of pyridine and silver nitrate. The reaction mixture is stirred overnight. The precipitate formed is filtered with suction and washed once more with a small amount of cold methanol. The precipitate is taken up in methanol and acetic acid is added. The hydrogen sulfide gas is introduced into the suspension. After a 30 min introduction of H₂S, rinsing with argon takes place and the silver sulfide formed is filtered with suction. The solution is treated with care in the rotary evaporator but not to dryness. For purification, column chromatography with the solvent DCM:MeOH 98:2 follows.

| R = | 0.4 g | |
|---|---|---|
| Analysis: | | |
| Yield | 47.6% | |
| Rf value: | solvent dichloromethane:methanol 95:5 | 0.53 |
| | dichloromethane:methanol 9:1 | 0.67 |
| ESI(−)MS | C₁₇H₁₇N₂O₅S | |
| Calculated | 361.39 | |
| Found | 360.9 | |

| | |
|---|---|
| NMR: | ¹H-NMR(250 MHz, DMSO-d6): δ = 11.38(br. s, 1H, N-H), |
| | 8.04-8.02(m, 2H, ortho-H benzoyl), 7.7-7.53(m, 3H, meta- and para-H |
| | benzoyl), 6.3-6.24(dd, 1H, H-1′), 5.46(m, 1H, H-3′), 4.21(m, |
| | 1H, H-4′), 3.32(s, 3H, OCH₃), 2.95-2.90(m, 1 H, H-5′), |
| | 2.66-2.60(m, 1H, H-5″), 2.51(m, 2 H, H-2′ and H-2″), 1.83 |
| | (d 3H, CH₃-T) |

### 5′-O-(4,4′-dimethoxytrityl)-thymidylyl-(3′→5′)-3′-O-benzoyl-2′deoxy-thymidine (9)

Size of batch:

| | |
|---|---|
| 0.361 g of 5′-thiol-3′-O-benzoyl-2′-deoxythymidine (8) | (1 mmole) |
| 0.745 g of T-amidite | (1 mmole) |
| 0.38 g of benzimidazol triflate | (1.4 mmole) |
| 5 ml of DCM | |
| 5 ml of ACN | |
| oxidation | |
| 0.876 g of tert. butyl ammonium periodate | (2 mmole) |

Experimental Execution:

5′-Thiol-3′-O-benzoyl-2′-deoxythymidine (8), T-amidite, benzimidazol triflate are dried in a pump vacuum (1 day) . The reaction is initiated by the addition of acetonitrile:dichloromethane. The solution is stirred for 1 h at room temperature and no residual educt can be detected by DC (LM DCM:MeOH 9:1). This is followed by the addition of the oxidising agent in 8 ml of DCM. The reaction solution is stirred for 8 min and subsequently diluted with a four fold quantity of DCM. Processing takes place by extraction with 5% sodium sulfite and 5% monosodium carbonate solution. The organic phase is dried over magnesium sulfate and treated in the rotary evaporator. For purification, column chromatography is then carried out with the solvent 0. 5 l of 1% MeOH in DCM, 0.5 l of 2% MeOH in DCM and 0.5 l of 3% MeOH in DCM.

| R = | 0.719 g | |
|---|---|---|
| Analysis: | | |
| Yield | 70.5% | |
| Rf value: | solvent dichloromethane:methanol 95:5 | 0.096 |
| | dichloromethane:methanol 9:1 | 0.32 |
| ESI(−)MS | C₅₁H₅₂N₅O₁₄PS | |
| Calculated | 1022.03 | |
| Found | 1020.8 | |

| | | |
|---|---|---|
| NMR | ³¹P-NMR(CDCl₃): | 17.26, 17.22 ppm |

### 5′-S-(4,4′-dimethoxytrityl)-thymidylyl-(3′→5′) -2′-deoxythymidine (10)

Size of batch:

0.586 g of 5′-O-(4,4′-dimethoxytrityl)-thymidylyl-(3′→5′)-3′-OBzl-2′-deoxythymidine (9) (0.573 mmole) 2 ml of 7 M ammonia in methanol 3 ml of methanol

Experimental Execution:

5′-O-(4,4′-dimethoxytrityl)-thymidylyl-(3′→5′) -3′-O-benzoyl.2′-deoxythymidine (9) is dissolved in ammoniac methanol and allowed to stand in a water bath overnight at 55° C. The complete cleavage of the benzoyl PG is verified by mass spectrometry. The solution is treated in the rotary evaporator and repeatedly co-evaporated with methanol.

| R = | 0.485 g | |
|---|---|---|
| Analysis: | | |
| Rf value: | solvent dichloromethane:methanol 9:1 | Initial spot |
| ESI(−)MS | C₄₁H₄₄N₄O₁₃PS | |
| Calculated | 863.86 | |
| Found | 863.6 | |

### 5′-O-(4,4′-dimethoxytrityl)-thymidylyl-(3′→5′)-3′-O-[(2-cyanoethyl)-N,N-diisopropylamidophosphoramidite]-thymidine (11)

Size of batch:

| | |
|---|---|
| 0.485 g of 5′-S-(4,4′-dimethoxytrityl)-thymidylyl-(3′→5′)- | (0.561 mmole) |
| 2′-deoxythymidine (10) | |
| 0.16 g of phosphorous acid mono-(2-cyanoethylester)- | (0.674 mmole) |
| diisopropylamide chloride | |
| 0.384 ml of N-ethyl diisopropyl amine (2.244 mmole) | |

Experimental Execution:

5′-S-(4,4′-dimethoxytrityl)-thymidylyl-(3′→5′)-2′-deoxythymidine (10) is dried overnight in a pump vacuum. DCM:ACN and DIPEA are added under a protective gas atmosphere. The phosphitilation reagent is slowly added dropwise with ice cooling. After 1 h, no educt can be detected by DC (solvent 95:5). The reaction is quenched by the addition of 0.5 ml of butanol. The reaction solution is treated in the rotary evaporator and taken up in 7.5 ml of DCM:diethyl ether and pipetted into 150 ml of cold pentane. The precipitate formed is filtered with suction and dried.

| R = | 0.556 g (Yield 93%) | |
|---|---|---|
| Analysis: | | |
| Yield | 93% | |
| Rf value: | solvent dichloromethane:methanol 9:1 | 0.12 |

| | |
|---|---|
| ESI(−) MS | C₅₀H₆₁N₆₀O₁₄P₂S |
| Calculated | 1064.08 |
| Found | 1063.8 |
| NMR: | ¹H-NMR (CDCl₃): δ=140.82, 140.75; 15.99, 15.85 |

### Example 13

### Preparation of 5′-S-9-[4-methoxyphenyl)xanthene-9-yl]-mercapto-2′-deoxythymidine-3′-O-(2-cyanoethyl, N,N′-diisopropyl phosphite)

The preparation of 5′-S-9-[4-methoxyphenyl)xanthene-9-yl]-mercapto-2′-deoxythymidine-3′-O-(2-cyanoethyl, N,N′-diisopropyl phosphite) is accomplished according to Example 13 using the synthesis scheme shown in FIG. 11.

### Preparation of 9-(4-methoxyphenyl)xanthene-9-ol (3):

Size ot batch:

| | | |
|---|---|---|
| 14.7 g | magnesium chips | (0.6 mole) |
| 75 ml | 4-bromanisol (1) | (0.6 mole) |
| 53.5 g | xanthene-9-one | (0.27 mole) |
| 300 ml | diethyl ether | |

Experimental Execution:

Magnesium chips are suspended in 100 ml of diethyl ether and a solution of bromanisol (1) in 100 ml of diethyl ether is slowly added dropwise. As soon as the Grignard reaction begins, the reaction solution is cooled to 15° C. The residual ethereal bromanisol solution is then added dropwise within 20 min such that the reaction solution boils slightly. On completion of the addition, refluxing is continued for a further hour, followed by cooling to room temperature. Xanthene-9-one is then added in small portions within 10 min and a further 100 ml diethyl ether are added to the reaction mixture which is then heated to boiling. The solution is kept boiling for a further 2 hours. After cooling, the reaction mixture is diluted—contrary to the instructions of J. H. Mariott, M. Mottahedeh, C. B. Reese in Carbohydrate Research (1991) 216:257-69: “Synthesis of 2′-thioadenosine”—with diethyl ether in order to be able to filter the resultant precipitate with suction. The filtered precipitate is then washed with diethyl ether and dried. The finely ground solid is introduced into 300 ml of concentrated HCl (cooling on an ice bath). The resulting blackish-red solution is added dropwise to 2 l of ice water. The aqueous solution is extracted three times with 650 ml of dichloromethane. The organic phases are combined and extracted three times with 1.5 l of saturated monosodium carbonate solution and twice with 1 l of water. The organic phase is dried over magnesium sulfate and treated in the rotary evaporator. Recrystallisation from cyclohexane takes place.

| R = | 56.43 g | |
|---|---|---|
| Analysis: | | |
| Yield | 68% | |
| Rt value: | solvent hexane:acetic acid ethyl ester 3:1 | 0.49 |

| | |
|---|---|
| ESI(+)MS | C₂₀H₁₆O₃ |
| Calculated | 304.33 |
| Found | 286.9 (cleavage of the hydroxyl group) |
| NMR: | ¹³C-NMR (CDCl₃): 55.15, 70.15, 113.23, 116.33, 123.47, |
| | 127.36, 128.87, 128.91, 140.40, 149.66, 158.21 |

### Preparation of 9-(4-methoxyphenyl)xanthene-9-thiol (AXT) (4)

Size of batch:

| | | |
|---|---|---|
| 20.8 g | dichloroacetic acid | (0.25 mole) |
| 38.6 g | 9-(4-methoxyphenyl)xanthene-9-ol (3) | (0.127 mole) |
| 1100 ml | dichloromethane | |

Experimental Execution:

H₂S is introduced for 15 minutes into a solution of dichloroacetic acid in 500 ml of dichloromethane with ice cooling. A solution of 9-(4-methoxyphenyl)xanthene-9-ol (3) in 600 ml of dichloromethane is added dropwise within 1 hour while further H₂S is introduced. On completion of the addition, H₂S is introduced for a further 15 min. By means of the subsequent introduction of argon, excess H₂S is to be displaced. Subsequently, the reaction solution is extracted three times with 700 ml of saturated monosodium carbonate solution and twice with 600 ml of water. The organic phase is dried over magnesium sulfate and treated in the rotary evaporator. Recrystallisation from cyclohexane takes place.

| R = | 17.02 g | |
|---|---|---|
| Analysis: | | |
| Yield | 80.3% | |
| Rf value: | solvent hexane:acetic acid ethyl ester 3:1 | 0.72 |

| | |
|---|---|
| ESI(+)MS | C₂₀H₁₆O₂S |
| Calculated | 320.33 |
| Found | 321.0 |
| NMR | ¹³C-NMR (CDCl₃): 51.35, 55.25, 113.20, 116.40, 123.42, |
| | 128.32, 129.10, 129.43, 130.24, 138.23, 149.62, |
| | 159.49 |

### Preparation of 5′-chloro-2′deoxythymidine (2)

Size of batch:

| | | |
|---|---|---|
| 9.6 g | 2′-deoxythymidine | (40 mmole) |
| 14 g | triphenylphosphine | (54 mmole) |
| 20 ml | carbon tetrachloride | (200 mmole) |
| 200 ml | DMF | |

Experimental Execution:

Thymidine and triphenylphosphine are dried together overnight in a pump vacuum. Carbon tetrachloride and DMF are added under a protective gas atmosphere. A slight temperature increase can be observed. The reaction solution is stirred for 24 h at room temperature. The reaction is quenched by adding methanol. The solvent is removed and the oily residue is recrystallised from methanol. The mother liquor still contains product, which however cannot be isolated.

| R = | 7.56 g | |
|---|---|---|
| Analysis: | | |
| Yield | 72.6% | |
| Rf value: | solvent dichloromethane:methanol 9:1 | 0.38 |

| | |
|---|---|
| ESI(+, −) MS | (C₁₇H₂₀N₂O₇S) |
| Calculated | 260.68 |
| Found | (+) 261.9 |
| | (−) 294.9 |
| NMR: | ¹H-NMR(250MHz, DMSO-d6): 11.38(br. s, 1H, N-H), |
| | 7.55(d, 1H, H-6), 6.22(t, 1H, H-1′), 4.25(m, 1H, 3-H′), |
| | 4.24(m, 3H, H-4′+H-5′+H-5″), 3.34(d, 1H, 3′-OH), |
| | 2.32-2.21(m, 1H, H-2′), 2.14-2.06(m, 1H, H-2″), |
| | 1.8(s, 3H, CH3-T) |

### Preparation of 5′-S-[9-(4-methoxyphenyl)xanthene-9-yl]-2′-deoxythymidine (5)

Size of batch:

| | | |
|---|---|---|
| 4.4 g | 5′-chloro-2′-deoxythymidine (2) | (17.0 mmole) |
| 8.17 g | 9-(4-methoxyphenyl)xanthene-9-thiol (4) | (25.5 mmole) |
| 2.42 ml | 1,1,3,3-tetramethyl guanidine | (18.92 mmole) |
| 150 ml | DMSO | |

Experimental Execution:

5′-Chloro-2′-deoxythymidine and 9-(4-methoxyphenyl)xanthene-9-thiol (4) are dried together overnight in a pump vacuum. DMSO is added under a protective gas atmosphere and 1,1,3,3-tetramethyl guanidine is slowly added dropwise to the solution. After three hours, no residual educt can be detected by DC (solvent DCM:MeOH 95:5). The reaction solution is poured into 1 l of cooled dichloromethane and the organic solution is extracted with 800 ml of saturated monosodium carbonate solution (careful: pressure). The aqueous phase is re-extracted with 200 ml of dichloromethane. The organic phases are combined and extracted four times with 400 ml of water and dried over magnesium sulfate.

To purify the compound, column chromatography is subsequently carried out with the solvent DCM:MeOH 97:3.

| R = | 7.1 g | |
|---|---|---|
| Analysis: | | |
| Yield | 95% | |
| Rf value: | solvent dichloromethane:methanol 95:5 | 0.183 |
| | dichloromethane:methanol 9:1 | 0.46 |

| | |
|---|---|
| ESI(−)MS | C₃₀H₂₈N₂O₆S |
| Calculated | 544.55 |
| Found | 543.3 |
| NMR | ¹H-NMR(250 MHz, CDCl₃): δ=8.82(br. s, 1H, N-H), |
| | 7.38-7.35(m, 2H, aromatic), 7.2-7.02(m, 7H, aromatic), |
| | 6.9-6.93(m, 2H, aromatic), 6.81-6.78(m, 2H, aromatic), |
| | 6.06-6.01(dd, 1H, H-1′), 3.86-3.82(m, 1H, H-4′), 3.73(s, |
| | 3H, O-CH3), 3.6-3.52(m, 1H, H-3′), 2.21-2.11(m, 2H, H-5′, |
| | H-5″), 1.9-1.79(m, 2H, H-2′, H-2″), 1.84(d, 3H, CH3-T) |

### 5′-S-[9- (4-methoxyphenyl)xanthene-9-yl]-mercapto-2′-deoxythymidine-3′-O-(2-cyanoethyl, N,N′-diisopropyl phosphite) (6)

Size of batch:

| | | |
|---|---|---|
| 0.75 g | 5′-S-(9-(4-methoxyphenyl)xanthene-9-yl)-2′- | (1.38 mmole) |
| | deoxythymidine (5) | |
| 0.392 ml | phosphorous mono-(2-cyanoethylester) | (1.65 mmole) |
| | diisopropylamide chloride | |
| 0.706 ml | N-ethyl diisopropyl amine | (4.13 mmole) |
| 5 ml | DCM | |
| 5 ml | ACN | |

Experimental Execution:

5′-S-[9- (4-methoxyphenyl)xanthene-9-yl]-2′-deoxythymidine (5) is dried overnight in a pump vacuum. DCM:ACN and DIPEA are added under a protective gas atmosphere. The phosphitilation reagent is slowly added dropwise with ice cooling. After 1 h, no further educt can be detected by DC (solvent DCM:MeOH 95:5). The reaction is quenched by the addition of 0.5 ml of butanol. The reaction solution is treated in the rotary evaporator and taken up in 10 ml of DCM:diethyl ether and pipetted into 200 ml of cold pentane. The resultant precipitate is filtered with suction and dried.

| | | |
|---|---|---|
| R = | 0.96 g | |
| Analysis: | | |
| Yield | 94% | |
| Rf value: | solvent dichloromethane:methanol 9:1 | 0.77 |
| | dichloromethane:methanol 95:5 | 0.51 and 0.44 |
| ESI(−)MS | C₃₉H₄₅N₄O₇S | |
| Calculated | 744.77 | |
| Found | 743.8 | |
| NMR | ³¹P-NMR(400MHz, CDCl₃): | |
| | 150.21, 149.94 ppm | |

### Example 14

### Synthesis of Amidites Protected by 5′-S-dimethoxytrityl

The preparation of amidites protected by 5′-S-dimethoxytrityl is carried out according to Example 14 using the synthesis scheme of FIG. 12.

### 4,4′-dimethoxytriphenylmethanol (14)

Size of batch:

| | | |
|---|---|---|
| 8.26 g | magnesium chips | (0.34 mol) |
| 43.25 ml | p-bromanisol (12) | (0.35 mol) |
| 20 ml | benzoic acid methyl ester (13) | (0.16 mol) |
| 30 ml | THF | |

Experimental Execution:

Magnesium chips are overlaid with 30 ml of THF under a protective gas atmosphere. Subsequently, p-bromanisol (12) is slowly added dropwise until the solution begins to boil spontaneously. On completion of the addition, the solution is refluxed for 1 h. Following the dropwise addition of benzoic acid methyl ester (13) in 30 ml of THF at room temperature, the reaction solution is refluxed for 90 min. After hydrolysis with saturated ammonium chloride solution, the phases are separated. The organic phase is washed with water and the aqueous phase is subsequently re-extracted with toluene. The organic phases are combined and dried over magnesium sulfate. After spinning, the crude product is taken up in a small amount of dichloromethane and a column chromatography is carried out (solvent dichloromethane).

| | | |
|---|---|---|
| R = | 48.7 g | |
| Analysis: | | |
| Yield | 95% | |
| Rf value: | solvent hexane:acetic acid ethyl ester 3:1 | 0.43 |
| ESI(+)MS | C₂₁H₂₀O₃ | |
| Calculated | 320.39 | |
| Found | 304.2 (—OH group) | |

| | |
|---|---|
| NMR | ¹H-NMR(250MHz, CDCl₃): 7.27-7.06(m, 9H, aromatic), |
| | 6.78-6.72(m, 4H, aromatic), 3.77(s, 6H, 2 × OCH₃) |

### 4,4′-Dimethoxytriphenylmethane thiol (15)

Size of batch:

| | | |
|---|---|---|
| 30.95 g | dichloroacetic acid | (0.24 mol) |
| 38.44 g | 4,4′-dimethoxytriphenylmethanol (14) | (0.12 mol) |
| 800 ml | dichloromethane | |

Experimental Execution:

Hydrogen sulfide is introduced for 15 min into a solution of dichloroacetic acid in 400 ml of dichloromethane with ice cooling. A solution of 4,4′-dimethoxytriphenylmethanol (14) in 400 ml of dichloromethane is added dropwise within 1 h while the introduction of hydrogen sulfide is continued. On completion of the addition, hydrogen sulfide is continued to be introduced for a further 15 min. As a result of the subsequent introduction of argon, excess hydrogen sulfide is to be displaced. The reaction solution is then extracted three times with 700 ml of saturated monosodium carbonate solution (careful: pressure) and twice with 600 ml of water. The organic phase is dried over magnesium sulfate and treated in the rotary evaporator. The crude product is dissolved in a small amount of dichloromethane, and hexane is added; the formation of a precipitate can be observed during this process. The solution is allowed to stand for 4 h with ice cooling, the solid is filtered with suction and dried.

| R = | 34.31 g (Yield 85%) | |
|---|---|---|
| Analysis: | | |
| Rf value: | solvent Hexane:acetic acid ethyl ester 3:1 | 0.66 |
| MALDI-MS | C₂₁H₂₀O₂S | |
| Calculated | 336.45 | |
| Found | 336.45 | |

| | |
|---|---|
| NMR | ¹H-NMR(250MHz, CDCl₃): 7.24-7.06(m, 9H, |
| | aromatic), 6.75-6.69(m, 4H, aromatic), 3.72(s, 6H, |
| | 2 × OCH₃) |

Introduction of the corresponding protective base groups:

### N⁶-benzoyl-2′-deoxyadenosine

Size of batch:

| | | |
|---|---|---|
| 12.89 g | 2′-deoxyadenosine | (51.3 mmole) |
| 32.4 ml | trimethyl chlorosilane | (256 mmole) |
| 27.8 ml | benzoyl chloride | (256 mmole) |
| 400 ml | pyridine | |

Aqueous ammonia solution
Acetic acid ethyl ester
Water

Experimental Execution:

2′-deoxyadenosine is dried overnight in a pump vacuum over phosphorus pentaoxide. The solid is suspended in pyridine and the reaction mixture cooled to 0° C. Trimethyl chlorosilane is added dropwise and benzoyl chloride is added dropwise after stirring for 30 min and stirring is continued for a further 2 h at room temperature. The reaction mixture is cooled to 0° C., 100 ml of water and 100 ml of ammonia solution are added and the mixture is concentrated after 30 min in the rotary evaporator. The remaining residue is dissolved in 350 ml of hot water and extracted twice with 150 ml of acetic acid ethyl ester. The acid ester phases are combined and extracted with 100 ml of hot water. The aqueous solution is allowed to stand overnight in the refrigerator while a precipitate is formed which is then filtered with suction and dried in the desiccator over phosphorus pentaoxide.

| R = | 11.4 g | |
|---|---|---|
| Analysis: | | |
| Yield | 63% | |
| Rf value: | solvent dichloromethane:methanol 9:1 | 0.33 |
| ESI(−)MS | C₁₇H₁₇N₅O₄ | |
| Calculated | 355.35 | |
| Found | 354.1 | |

| | |
|---|---|
| NMR | ¹H-NMR(250MHz, DMSO-d6): δ=8.76-8.72(2s, |
| | per 1H, H-8 and H-2), 8.06-8.02(m, 2H, ortho-H |
| | benzoyl), 7.64-7.51(m, 3H, meta- and para-H |
| | benzoyl)6.53-6.47(m, 1H, H-1′), 4.50-4.46(m, 1H, |
| | H-3′), 3.95-3.90(m, 1H, H-4′), 3.68-3.52(m, 2H, |
| | H-5′+H-5″), 2.85-2.75(m, 1H, H-2′), 2.43-2.34(m, |
| | 1H, H-2″) |

### N⁴-benzoyl-2′-deoxycytidine

Size of batch:

| | | |
|---|---|---|
| 7.91 g | 2′-deoxycytidine × HCl | (30 mmole) |
| 19 ml | trimethyl chlorosilane | (150 mmole) |
| 17.4 ml | benzoyl chloride | (150 mmole) |
| 300 ml | pyridine | |

Aqueous ammonia solution
Acetic acid ethyl ester
Water

Experimental Execution:

2′-deoxycytidine is dried overnight in a pump vacuum over phosphorus pentaoxide. The solid is dissolved in pyridine and the reaction mixture is cooled to 0° C. Trimethyl chlorosilane is added dropwise and benzoyl chloride is added dropwise after stirring for 30 min. Stirring is continued for a further 2 h at room temperature. The reaction mixture is cooled to 0° C. and 60 ml of water and 60 ml of ammonia solution are added and after 30 min the mixture is concentrated in the rotary evaporator. The remaining residue is dissolved in 300 ml of hot water and extracted twice with 100 ml of acetic acid ethyl ester. The acid ester phases are combined and extracted with 100 ml of hot water. The aqueous solution is allowed to stand overnight in the refrigerator while a precipitate is formed which is filtered with suction and dried in the desiccator over phosphorus pentaoxide.

| R = | 7.3 g | |
|---|---|---|
| Analysis: | | |
| Yield | 73.2% | |
| Rf value: | solvent dichloromethane:methanol 9:1 | 0.27 |
| ESI(−)MS | C₁₆H₁₇N₃O₅ | |
| Calculated | 331.33 | |
| Found | 330.0 | |

| | |
|---|---|
| NMR | ¹H-NMR(250MHz, DMSO-d6): δ=11.23(b, 1H, |
| | N-H), 8.41-8.39(d, 1H, H-6), 8.03-8.00(m, 2H, |
| | ortho-H benzoyl), 7.67-7.49(m, 3H, meta- and |
| | para-H benzoyl)7.36-7.33(d, 1H, H-5), |
| | 6.18-6.13(m, 1H, H-1′), 5.28-5.26(d, 1H, 3′-OH), |
| | 5.09-5.05(m, 1H, 5′-OH), 4.29-4.22(m, 1H, H-3′), |
| | 3.92-3.87(m, 1H, H-4′), 3.71-3.57(m, 2H, |
| | H-5′+H-5″), 2.38-2.29(m, 1H, H-2′), |
| | 2.13-2.02(m, 1H, H-2″) |

### N²-Isobutyryl-2′deoxyguanosine

Size of batch:

| | | |
|---|---|---|
| 13.36 g | 2′-deoxyguanosine | (50 mmole) |
| 31.6 ml | trimethyl chlorosilane | (250 mmole) |
| 41.6 ml | isobutyric anhydride | (250 ml) |
| 400 ml | pyridine | |

Experimental Execution:

2′-deoxyguanosine is dried overnight in a pump vacuum over phosphorus pentaoxide. The solid is suspended in pyridine and the reaction mixture is cooled to 0° C. Trimethyl chlorosilane is added dropwise while the solid is dissolved. After stirring for 30 min, isobutyric anhydride is added. Stirring is continued for further 2 h at room temperature. The reaction mixture is cooled to 0° C., 100 ml of water and 100 ml of ammonia solution are added and the mixture concentrated after 30 min in the rotary evaporator. The remaining residue is dissolved in 300 ml of hot water and extracted twice with 200 ml of acetic acid ethyl ester. The acid ester phases are combined and extracted with 50 ml of hot water. The aqueous solution is allowed to stand overnight in the refrigerator while a precipitate is formed, which is then filtered with suction and dried in the desiccator over phosphorus pentaoxide.

| R = | 12.4 g | |
|---|---|---|
| Analysis: | | |
| Yield | 73.3% | |
| Rf value: | solvent dichloromethane:methanol 9:1 | 0.37 |

| | |
|---|---|
| ESI(−)MS | C₁₄H₁₉N₅O₅ |
| Calculated | 337.34 |
| Found | 336.0 |
| NMR | ¹H-NMR(250MHz, DMSO-d6) δ=8.16(s, 1H, H-8) |
| | 6.25-6.19(m, 1H, H-1′), 5.33(d, 1H, 3′-OH), |
| | 5.33-5.31(d, 1H, 5′-OH), 4.39-4.38(m, 1H, H-3′), |
| | 3.87-3.83(m, 1H, H-4′), 3.61-3.34(m, 2H, H-5′+H-5″), |
| | 2.84-2.73(m, 1H, CH-isobutyryl), 2.62-2.54(m, 1H, H-2′), |
| | 2.33-2.24(m, 1H, H-2″), 1.14-1.07(dd, 6H, CH₃-isobutyryl) |

General operating instructions for the 5′-O-mesylation and 5′-O-tosylation of 2′-deoxynucleotides (16 a-g) 5′-O-mesylation

Size of batch:

| | |
|---|---|
| 2′-deoxynucleotide | (4 mmole) |
| Methanesulfonyl chloride | (4 mmole) 0.372 ml |
| 40 ml pyridine | |

Experimental Execution:

The corresponding 2′-deoxynucleotide is suspended in pyridine (thymidine gives a clear solution) and the reaction mixture is cooled to −20° C. Methanesulfonyl chloride is added dropwise over a period of 30 min. The reaction batch is allowed to stand overnight at −20° C. The progress of the reaction is checked by DC (solvent dichloromethane:methanol 9:1). On completion of the reaction, quenching with methanol and concentration in the rotary evaporator are carried out. For purification, column chromatography is then carried out with the corresponding dichloromethane: methanol mixtures. During this process, it can be observed that the compounds are poorly soluble in pure dichloromethane.

### 5′-O-tosylation

Size of batch:

| | |
|---|---|
| 2′-deoxynucleotide | (4 mmole) |
| tosyl chloride | (4.8 mmole) |
| 40 ml of pyridine | |

Experimental Execution:

The corresponding 2′-deoxynucleotide is suspended in pyridine (thymidine gives a clear solution) and the reaction mixture is cooled to 0° C. Tosyl chloride is added batchwise over a period of 15 min. The reaction batch is stirred with ice cooling. The progress of the reaction is checked by DC (solvent dichloromethane:methanol 9:1). On completion of the reaction, quenching with methanol and concentrating in the rotary evaporator are carried out. For purification, column chromatography is then carried out with dichloromethane:methanol mixtures.

### T-nucleoside (16 a-c)

| Analysis: | 5′-O-tosyl-2′deoxythymidine (16a) | |
|---|---|---|
| Yield | 68% | |
| Rf value: | solvent dichloromethane:methanol 9:1 | 0.29 |

| | |
|---|---|
| ESI(+)-MS | (C₁₇H₂₀N₂O₇S) |
| Calculated | 396.42 g/mol |
| Found | 397.1 g/mol |
| Melting point | 168-169° C. |
| NMR | ¹H-NMR(250MHz, CDCl₃): 11.31(br. s, 1H, N-H), |
| | 7.82(d, 2H, aromatic, AA′BB′), |
| | 7.44(d, 2H, aromatic, AA′BB′), |
| | 7.36(d, 1H, H-6), 6.14(t, 1H, H-1′), |
| | 5.57(d, 1H, 3′-OH), 4.21(m, 3H, H-5′+H-5″+H-3′), |
| | 3.85(m, 1H, H-4′), 2.40(s, 3H, CH₃-tosyl), |
| | 2.10(m, 2H, H-2′+H-2″), 1.75(s, 3H, CH₃-T) |

### Analysis of 5′-O-mesyl-2′-deoxythymidine (16b)

| Yield | 64% | |
|---|---|---|
| Rf value: | solvent dichloromethane:methanol 9:1 | 0.32 |

| | |
|---|---|
| ESI(+)-MS | (C₁₁H₁₆N₂O₅S) |
| Calculated | 320.32 |
| Found | 321.0 |
| | 343.0 (+Na-salt) |
| NMR | ¹H-NMR(250MHz, DMSO-d6): 11.32(br. s, 1H, N-H), |
| | 7.49(d, 1H, H-6), 6.26-6.20(t, 1H, H-1′), |
| | 5.49(d, 1H, 3′-OH), 4.21-4.35(m, 2H, H-5′+H-5″), |
| | 4.29-4.27(m, 1H, 3-H′), 4.01-3.96(m, 1H, H-4′), |
| | 3.26(s, 3H, CH₃-mesyl), 2.53-2.50(m, 1H, H-2′), |
| | 2.27-2.07(m, 1H, H-2′), 1.79(s, 3H, CH₃-T) |
| | 5′-chloro-2′deoxythymidine (16c) |

The reaction is carried out as described for the preparation of 5′-S-9-[4-methoxyphenyl)xanthene-9-yl]-mercapto-2′-deoxythymidine-3′-O-(2-cyanoethyl, N,N′-diisopropyl phosphite).

| Analysis: | | |
|---|---|---|
| Yield | 72.6% | |
| Rf value: | solvent dichloromethane:methanol 9:1 | 0.38 |
| ESI(+, −)-MS | (C₁₇H₂₀N₂O₇S) | |
| Calculated | 260.68 | |
| Found | (+) 261.9 | |
| | (−) 294.9 | |
| NMR | ¹H-NMR(250MHz, DMSO-d6): 11.38(br. s, 1H, N-H), | |
| | 7.55(d, 1H, H-6), 6.22(t, 1H, H-1′), 4.25(m, 1H, 3-H′), | |
| | 4.24(m, 3H, H-4′+H-5′+H-5″), | |
| | 3.34(d, 1H, 3′-OH), 2.32-2.21(m, 1H, H-2′), | |
| | 2.14-2.06(m, 1H, H-2″), 1.8(s, 3H, CH₃-T) | |
| | A-nucleosides (16d-e) | |
| Analysis: | 5′-O-tosyl-N⁶-benzoyl-2′-deoxyadenosine (16d) | |
| Yield | 39% | |
| Rf value: | solvent dichloromethane:methanol 9:1 | 0.42 |
| MALDI-MS | C₂₄H₂₃N₅O₆S | |
| Calculated | 509.43 | |
| Found | 509.69 | |
| NMR | ¹H-NMR (250MHz, CDCl₃): | |
| | δ=8.68-8.56(m, 2H, H-8 and H-2), | |
| | 7.97-7.91(m, 2H, ortho-H benzoyl), | |
| | 7.58-7.27(m, 7H, meta- and para-H benzoyl, H-tosyl), | |
| | 6.42-6.37(m, 1H, H-1′), | |
| | 4.79-4.69(m, 1H, H-3′), 4.55-4.51(m, 1H, H-4′), | |
| | 2.93-2.76(m, 2H, H-5′+H-5″), | |
| | 2.59-2.47(m, 2H, H-2′+H-2″), | |
| | 2.30(s, 3H, CH₃-tosyl) | |
| Analysis: | 5′-O-mesyl-N⁶-benzoyl-2′-deoxyadenosine | |
| | (16e) | |
| Yield | 79% | |
| Rf value: | solvent dichloromethane:methanol 9:1 | 0.33 |
| ESI(−)MS | C₁₈H₁₉N₅O₆S | |
| Calculated | 433.4 | |
| Found | 432.3 | |
| NMR | ¹H-NMR(250 MHz, DMSO-d6): δ=11.2 (s br. N-H), | |
| | 8.78-8.66(m, 2H, H-8 and H-2), | |
| | 8.08-8.04(m, 2H, ortho-H benzoyl), | |
| | 7.69-7.48(m, 3H, meta- and para-H benzoyl), | |
| | 6.59-6.53(m, 1H, H-1′), | |
| | 4.56-4.37(m, 2H, O-H and H-3′, 4.18-4.14(m, 1H, H-4′), | |
| | 3.37-3.27(m, 2H, H-5′+H-5″), 3.21-3.17(d, 3H, | |
| | H-mesyl), 2.99-2.88(m, 1H, H-2′), 2.47-2.41(m, 1H, | |
| | H-2″)G-nucleosides (16f) | |
| Analysis: | 5′-O-mesyl-N²-isobutyryl- | |
| | 2′deoxyguanosine (16f) | |
| Yield | 93% | |
| Rf value: | solvent dichloromethane:methanol 9:1 | 0.28 |
| ESI(−)MS | C₁₅H₂₁N₅O₇S | |
| Calculated | 415.43 | |
| Found | 414.0 | |
| NMR: | ¹H-NMR(250MHz, DMSO-d6) δ=11.62(s br., 1H, N-H), | |
| | 8.22-8.17(s, 1H, H-8), 6.31-6.25(m, 1H, H-1′), | |
| | 5.57-5.55(d, 1H, 3′-OH), | |
| | 4.46-4.31(m, 3H, H-3′+H-5′+H-5″), | |
| | 4.11-4.06(m, 1H, H-4′), 3.17(s, 1H, CH₃-mesyl), | |
| | 2.83-2.66(m, 2H, H-2′ and CH-isobutyryl), | |
| | 2.41-2.31(m, 1H, H-2″), | |
| | 1.15-1.07(dd, 6 H, CH₃-isobutyryl) | |
| | C-nucleoside (16g) | |
| Analysis: | 5′-O-mesyl-N⁴-benzoyl-2′deoxycytidine (16g) | |
| Yield | 48% | |
| Rf value: | solvent dichloromethane:methanol 9:1 | 0.28 |

| | |
|---|---|
| ESI(−) MS | C₁₇H₁₉N₃O₇S |
| Calculated | 409.42 |
| Found | 408.1 |
| NMR: | ¹H-NMR(250MHz, DMSO): δ=11.27(b, 1H, N-H), |
| | 8.33(d, 1H, H-6), 8.14-8.03(m, 2H, ortho-H benzoyl), |
| | 7.67-7.49(m, 3H, meta- and para-H benzoyl), |
| | 7.39-7.36(d, 1H, H-5), 6.24-6.19(m, 1H, H-1′), |
| | 5.56-5.54(d, 1H, 3′-OH), |
| | 4.49-4.39(m, 2H, H-5′ and H-5″), |
| | 4.31-4.25(m, 1H, H-3′), 4.13-4.08(m, 1H, H-4′), |
| | 3.26(s, 3H, CH₃-mesyl), 2.42-2.32(m, 1H, H-2), |
| | 2.25-2.08(m, 1H, H-2″) |

### General Operating Instructions for 5′-S-(4,4′-dimethoxytriphenyl)-mercapto-2′-deoxynucleotides (17 a-d)

Size of batch:

| | |
|---|---|
| 5′-X-nucleoside (16 a-g) | (1 mmole) |
| 4,4′-dimethoxytriphenyl methane thiol | (1.5 mmole) |
| 1,1,3,3-tetramethyl guanidine | (1.11 mmole) |
| 10 ml of DMSO | |

Experimental Execution:

5′-X-nucleoside (16 a-g) and 4,4′-dimethoxytriphenyl methane thiol (15) are dried together overnight in a pump vacuum. DMSO is added under a protective gas atmosphere and 1,1,3,3-tetramethyl guanidine is slowly added dropwise to the solution. After three hours, progress of the reaction is verified by DC (solvent DCM:MeOH 95:5). If the reaction is considered to be completed at that point, the reaction solution is poured into 300 ml of cooled dichloromethane and the organic solution is extracted with 150 ml of saturated monosodium carbonate solution (careful: pressure). The aqueous phase is re-extracted with 100 ml of dichloromethane. The organic phases are combined and extracted four times with 100 ml of water and dried over magnesium sulfate. For purification, column chromatography is then carried out with a solvent mixture of dichloromethane and methanol.

### Analysis: 5′-S-(4,4′-dimethoxytriphenyl)-2′-deoxythymidine (17a)

| Yield when | | |
|---|---|---|
| using | | |
| a. | 5′-Cl-2′-deoxythymidine (16c) | 63.6% |
| b. | 5′-O-tosyl-2′-deoxythymidine (16a) | 94% |
| c. | 5′-O-mesyl-2′-deoxythymidine (16b) | 97.5% |
| Rf value: | solvent dichloromethane:methanol 9:1 | 0.51 |
| ESI(−)MS | C₃₁H₃₂N₂O₆S | |
| Calculated | 559.66 | |
| Found | 559.5 | |
| NMR | ¹H-NMR(250MHz, CDCl₃): 8.81(br. S, 1H, N-H), | |
| | 7.64-7.56(m, 1H, H-6), 7.35-7.14(m, 9 H, aromatic), | |
| | 6.77-6.71(m, 4H, aromatic), 6.14-6.09(dd, 1H, H-1′), | |
| | 4.05-4.03(m, 1H, H-4′), 3.76-3.74(m, 1H, H-3′), | |
| | 3,7(s, 6H, 2×OCH₃), 2.6-2.53(m, 1H, H-5′), | |
| | 2.45-2.38(m, 1H, H-5″), 2.25-2.18(m, 2H, H-2′+H-2″), | |
| | 1.78(d, 3H, CH₃-T) | |
| Analysis: | 5′-S-(4,4′-dimethoxytriphenyl)-N⁶-benzoyl-2′- | |
| | deoxyadenosine (17b) | |
| Yield when | | |
| using | | |
| a. | 5′-O-tosyl-N⁶-benzoyl-2′-deoxyadenosine (16d) | 94% |
| b. | 5′-O-mesyl-N⁶-benzoyl-2′-deoxyadenosine | 41% |
| | (16e) | |
| Rf value: | solvent dichloromethane:methanol 9:1 | 0.45 |
| ESI(−)MS | C₃₈H₃₅N₅O₅S | |
| Calculated | 673.77 | |
| Found | 672.6 | |
| NMR | ¹H-NMR(250MHz, CDCl₃): δ=9.08(s, 1H, N-H), | |
| | 8.65, 8.22(s, 2H, H-2 and H-8), | |
| | 7.93-7.92(m, 2H, ortho-H-benzoyl), | |
| | 7.51-7.11(m, 12H, aromatic), | |
| | 6.74-6.68(m, 4H, aromatic), | |
| | 6.27-6.22(m, 1H, H-1′), 4.33-4.29(m, 1H, H-3′), | |
| | 3.77-3.74(m, 1H, H-4′), 3.69(s, 6H, 2×OCH₃), | |
| | 2.74-2.36(m, 4H, H-5′+H-5″+H-2′+H-2″) | |
| Analysis: | 5′-S-(4,4′-dimethoxytriphenyl)-N²-isobutyryl-2′- | |
| | deoxyguanosine (17c) | |
| Yield when | | |
| using | | |
| a. | 5′-O-mesyl-N²-isobutyryl-2′deoxyguanosine | 45% |
| | (16f) | |
| Rf value: | Solvent dichloromethane:methanol 9:1 | 0.43 |
| ESI(−)MS | C₃₅H₃₇N₅O₆S | |
| Calculated | 655.76 | |
| Found | 656.1 | |
| NMR: | ¹H-NMR(250MHz, CDCl₃) δ=10.24(s br., 1H, N-H), | |
| | 7.66(s, 1H, H-8), 7.29-7.02(m, 9H, aromatic), | |
| | 6.75-6.62(m, 4H, aromatic), | |
| | 5.98-5.93(t, 1H, H-1′), 4.98(s br., 1H, 3′-OH), | |
| | 4.63(m, 1H, H-3′), 4.09-4.01(m, 1H, H-4′), | |
| | 3.64(s, 6H, 2×OCH₃), | |
| | 2.89-2.84(m, 1H, CH-isobutyryl), | |
| | 2.53-2.45(m, 2H, H-5′ and H-5″), | |
| | 2.31(m, 2H, H-2′ and H-2″), | |
| | 1.21-1.16(dd, 6H, CH₃-isobutyryl) | |
| Analysis: | 5′-S-(4,4′-dimethoxytriphenyl)-N⁴-benzoyl-2′- | |
| | deoxycytidine (17d) | |
| Yield when | | |
| using | | |
| b. | 5′-O-mesyl-N⁴-benzoyl-2′deoxycytidine (16g) | 82% |
| Rf value: | solvent dichloromethane:methanol 9:1 | 0.44 |

| | |
|---|---|
| ESI(−)MS | C₃₅H₃₇N₅O₆S |
| Calculated | 649.77 |
| Found | 634.4 (cleavage of CH₃ groups) |
| NMR | ¹H-NMR(250 MHz, CDCl₃): δ=8.17-8.15(m, 1H, H-6), |
| | 7.86-7.04(m, 16H, ortho-H benzoyl, H-trityl), |
| | 7.81-6.77(m, 2H, aromatic), |
| | 6.06-6.01(d, 1H, H-1′), |
| | 3.90-3.83(m, 2H, H-3′ and H-4′), |
| | 3.73(s, 6H, 2×OCH₃), 2.57-2.29(m, 4H, |
| | H-5′+H-5″+H-2′+H-2″) |

| | |
|---|---|
| General operating instructions | 5′-S-(4,4′-dimethoxytriphenyl) |
| for 5′-S-(4,4′- | nucleosides (17 a-d) (1 mmole) |
| dimethoxytriphenyl)-mercapto- | |
| 2′-deoxynucleotide-3′-O-(2- | |
| cyanoethyl, N,N′-diisopropyl | |
| phosphites) (18 a-d) | |
| Size of batch: | |
| Phosphorous acid mono- | (1.2 mmole) |
| (2-cyanoethylester) | |
| diisopropylamide | |
| chloride | |
| N-ethyl diisopropyl amine | 4 ml of DCM |
| mmole) | 4 ml of ACN |

Experimental Execution:

5′-S-(4,4′-dimethoxytriphenyl) nucleoside (17 a-d) is dried overnight in a pump vacuum. DCM:ACN and DIPEA are added under a protective gas atmosphere. The phosphitilation reagent is slowly added dropwise with ice cooling. The reaction is monitored for approximately 1 h by DC (solvent 95:5). The reaction is quenched by adding 0.5 ml of butanol. The reaction solution is treated in the rotary evaporator and taken up in 6 ml of DCM:diethyl ether and pipetted into 120 ml of cold pentane. The precipitate formed is filtered with suction and is then dried. Depending on the degree of purity, column chromatography is then carried out.

| Analysis: | 5′-S-(4,4′-dimethoxytriphenyl)- | |
|---|---|---|
| | mercapto-2′-deoxythymidine-3′-O-(2-cyanoethyl, | |
| | N,N′-diisopropyl phosphite) (18a) | |
| Yield | 73% | |
| Rf value: | solvent dichloromethane:methanol 9:1 | 0.65 |
| ESI (+) MS | C₄₀H₄₉N₄O₇PS | |
| Calculated | 760.86 | |
| Found | 761.2 | |

| | |
|---|---|
| NMR | |
| ³¹P-NMR | CDCl₃+0.1% DIPEA |
| | 149.55, 149.35 ppm |

| | | |
|---|---|---|
| Analysis: | 5′-S-(4,4′-dimethoxytriphenyl)- | |
| | mercapto-N⁶-benzoyl-2′- | |
| | deoxyadenosine-3′-O-(2-cyanoethyl, | |
| | N,N′-diisopropyl phosphite) (18b) | |
| Yield | 68% | |
| Rf value: | solvent dichloromethane:methanol 9:1 | 0.90 |
| | dichloromethane:methanol 95:5 | 0.81 and 0.77 |
| ESI (+) MS | C₄₇H₅₂N₇O₆PS | |
| Calculated | 873.99 | |
| Found | 858.6 (cleavage of the CH₃ group) | |

| | |
|---|---|
| NMR | |
| ³¹P-NMR | CDCl₃+0.1% DIPEA |
| | 149.99, 149.74 ppm |

| | | |
|---|---|---|
| Analysis: | of 5′-S-(4,4′-dimethoxytriphenyl)- | |
| | mercapto-N⁶-isobutyryl-2′-deoxyguanosine- | |
| | 3′-O-(2-cyanoethyl, | |
| | N,N′-diisopropyl phosphite) (18c) | |
| Yield | 72% | |
| Rf value: | solvent dichloromethane:methanol 9:1 | 0.71 |
| | dichloromethane:methanol 95:5 | 0.37 |
| ESI(+)MS | C₄₄H₅₄N₇O₇PS | |
| Calculated | 856.01 | |
| Found | 856.6 | |

| | |
|---|---|
| NMR | |
| ³¹P-NMR | CDCl₃+0.1% DIPEA |
| | 150.13, 148.93 ppm |

| | | |
|---|---|---|
| Analysis: | 5′-S-(4,4′-dimethoxytriphenyl)- | |
| | mercapto-N⁴-benzoyl-2′-deoxycytidine- | |
| | 3′-O-(2-cyanoethyl, N,N′-diisopropyl | |
| | phosphite) (18d) | |
| Yield | 76.4% | |
| Rf value: | solvent dichloromethane:methanol 9:1 | 0.76 and 0.67 |
| ESI(+)MS | C₄₆H₅₂N₅O₇PS | |
| Calculated | 849.99 | |
| Found | 832.5 (cleavage of the CH₃ group) | |

| | |
|---|---|
| NMR | |
| ³¹P-NMR | CDCl₃+0.1% DIPEA |
| | 150.43, 149.99 ppm |

### Example 15

### Synthesis of the Photolabile Amidite 1-{5′-O-(4,4′-dimethoxytrityl)-2′-O-{[(2-nitrobenzyl)oxy]methyl}-β-L-ribofuranosyl}uracil 3′-[(2-cyanoethyl)-diisopropylphosphoramidite] (1-5)

The synthesis of 1-{5′-O-(4,4′-dimethoxytrityl)-2′-O-{[(2-nitrobenzyl)oxy]methyl}-β-L-ribofuranosyl}uracil 3′-[(2-cyanoethyl)-diisopropylphosphoramidite] (5) is accomplished according to this example using the synthesis scheme as shown in FIG. 13.

### 1) o-nitrobenzyl methyl thiomethyl ether (1-2) (C₉H₁₁NO₃S, Mw 213.26 )

A solution of o-nitrobenzyl alcohol (2.655 g, 17.34 mmole) and chloromethyl methyl sulfide (2.008 g, 20.08 mmole) in 12 ml of dry benzene was added dropwise within five minutes to a solution of silver nitrate (3.24 g, 19.07 mmole) and triethylamine (2.105 g, 20.8 mmole) in 20 ml of dry benzene. The solution was heated at 60° C. for 24 hours and then filtered through a dry Celite column. The solution was extracted with dichloromethane and washed with 3% aqueous phosphoric acid, saturated aqueous monosodium carbonate and water and is then dried. The residue was purified by column chromatography on silica gel (dichloromethane: n-hexane=2:3).

Product: 1.25 g (34%)

TLC (MC:Hex=1:2 ): Rf=0.15. ¹H-NMR (400 MHz,CDCl₃), δ in ppm: 2.19 (s,3H, —S—CH₃); 4.77 (s,2H, —O—CH₂—S); 4.98 (s,2H,Ar—CH₂—O) 7.42-8.07 (m,4H,Ar—H)

### 2) o-nitrobenzyl chloromethyl ether (1-3) (C₈H₈ClNO₃, Mw 201.61 )

Freshly distilled sulfur chloride (1.57 g, 11.35 mmole) in 10 ml of dried dichloromethane was added dropwise at room temperature within 10 minutes to a solution of pure and dry o-nitrobenzyl chloromethyl ether (2.42 g, 11.35 mmole) in 20 ml of dry dichloromethane and is subsequently stirred for one hour. The solution was evaporated by means of a rotary evaporator. The residue was distilled by means of a bulb tube oven at 100 to 110° C. and 0.05 torr. The product cannot be stored for a long period of time without distillation.

Product: 2.09 g (91%)

TLC (MC): Rf=0.07. ¹H-NMR (250 MHz,CDCl₃), δ in ppm:5.15 (s,2H,Ar—CH₂—O—CH₂—); 5.63 (s,2H—O—CH₂—Cl); 7.45-8.12 (m,4H,Ar—H). MS(ESI)=217.5 [M+NH₃]⁺

### 3) 1-{5′-O-(4,4′-dimethoxytrityl)-2′-O-{[(2-nitrobenzyl)oxy]methyl}-β-L-ribofuranosyl}uracil(1-4) and 1-{5′-O-(4,4′-dimethoxytrityl)-3′-O-{[(2-nitro-benzyl)oxy]methyl}-β-L-ribofuranosyl}uracil(1-4-1) (C₃₈H₃₇N₃O₁₁, Mw 711.72 )

Ethyl diisopropyl amine (2.10 g, 16.25 mmole) and Bu₂SnCl₂ (1.18 g, 3.90 mmole) were added to substance A (compare FIG. 13) (1.773 g, 3.25 mmole) in 12 ml of 1,2-dichloroethane. The solution was stirred at room temperature for 90 minutes under argon and subsequently heated at 70° C.

o-Nitrobenzyl methyl chloromethyl ether was added to the solution at 70° C. After 30 minutes, the mixture was extracted with dichloromethane, washed with saturated aqueous monosodium carbonate and dried. The residue was purified by column chromatography on silica gel (ethyl acetate: n-hexane=3:2, subsequently 1:9).

Product: substance-4 1.02g (44%), substance-4-1 0.78g (33.7%)

TLC ( Hex:EtOAc 32 1:9 ) Rf=0.61 (substance 4), 0.36 (substance 4-1). ¹H-NMR (1-4, 400 MHz,CDCl₃), δ in ppm: 2.67 (br.d,OH—C(3′); 3.52 (dd,H—C(5′); 3.55 (dd,H′—C(5′)); 3.79 (s′2MeO); 4.10(br.d,OH—C(3′)); 4.38 (dd,H—C(2′)); 4.55 (br.q,H—C(3′)); 5.03,5.09 (2d,OCH₂O); 5.04,5.18 (2d,ArCH₂O); 5.29 (d,H—C(5)); 6.04 (d,H—C(1′)); 6.82-6.87 (m,Ar-4H); 7.21-7.76 (m,Ar-12H); 7.94 (d,H—C(6)); 8.06-8.09 (m,Ar-1H); 9.02 (br.s,H—N(3)).

MS(ESI)=746.5[M+Cl]+

### 4) 1-{5′-O-(4,4′-dimethoxytrityl)-2′-O-{[(2-nitrobenzyl)oxy]methyl}-β-L-ribofuranosyl}uracil 3′-[(2-cyanoethyl)-diisopropylphosphoramidite] (1-5) ( C₄₇H₅₄N5O₉P, Mw 863.95 )

Ethyl diisopropyl amine (227 mg, 1.755 mmole) and phosphoramidite-B (91 mg, 0.385 mmole) were added to substance-4 (250 mg, 0.351 mmole) in 3 ml of a mixture of 1.5 ml of dichloromethane and 1.5 ml of acetonitrile. The solution was stirred for 1 hour under argon, quenched with butanol, extracted with ethyl acetate, washed with 5% monosodium carbonate and aqueous saturated sodium chloride, and then evaporated. The residue was dissolved in 3 ml of a mixture of 1.5 ml of dichloromethane and 1.5 ml of ether, subsequently added dropwise to 150 ml of cold pentane (ice bath) and filtered.

Product: 273 mg (90%)

TLC (Hex:EtOAc=2:3 ) Rf=0.5, 0.42. 1H-NMR (400 Hz,CDCl₃), δ in ppm: 1.02,1.11,1.13 (3d,12H,Me₂CH); 2.44,2.61 (2t,2H,OCH₂CH₂CN); 3.41-3.74 (m,5H,Me₂CH,H—C(5′),OCH₂CH₂CN) ; 3.78,3.79 (2s,6H,MeO); 3.91 (m,1H,OCH₂CH₂ON); 4.21 (dt,0.5H,H—C(4′)); 4.27 (dt,0.5H,H—C(4′)); 4.45 (dd,0.5H,H—C(2′)); 4.51 (br.t,0.5H,H—C(2′)); 4.53-4.58 (m,1H,H—C(3′)); 5.00-5.07 (m,4H,OCH₂O,ArCH₂); 5.19,5.23 (2d,1H,H—C(5)); 6.06 (d,0.5H,H—C(1′)); 6.10 (d,0.5H,H—C(1′)); 6.81-6.85 (m,Ar-4H); 7.22-7.45 (m,Ar-10H); 7.60,7.78 (2m,Ar-2H); 7.87,7.95 (2d,1H,H—C(6)); 8.06 (m,Ar-1H); 8.68 (br.s,1H,H—N(2)). 31P-NMR(162 MHz): 151.4, 150.4 MS(ESI)=887 [M+Na]⁺

### Example 16

### Synthesis of the Photolabile Amidite 1-{5′-O-(4,4′-dimethoxytrityl)-2′-O{[(1-(o-nitrobenzyl)ethyl)oxy]methyl}-β-L-ribofuranosyl}uracil 3′-[(2-cyanoethyl)-diisopropylphosphoramidite] (2-6)

The synthesis of 1-{5′-O-(4,4′-dimethoxytrityl)-2′-O-{[(1-(o-nitrobenzyl)ethyl)oxy]methyl}-β-L-ribofuranosyl}uracil 3′-[(2-cyanoethyl)-diisopropylphosphoramidite] (2-6) was carried out in this example using the scheme shown in FIG. 14.

### 1) 1-(o-nitrophenyl)ethanol (2-2) ( C₈H₉NO₃, Mw 167.16 )

3 g NaBH₄ and 3 ml water were added to 10 g of aluminium oxide at room temperature. The mixture was stirred for 6 hours, subsequently dried for approximately 18 hours by means of a vacuum pump.

o-Nitroacetophenone in 60 ml of dry diethyl ether was added to NaBH₄-Alox in 60 ml of dry diethyl ether at room temperature and stirred for 2 hours. The solution was filtered on silica gel without column chromatography.

Product: 5.50 g (99.8%)

TLC (MC:Hex=3:1): Rf=0.14. ¹H-NMR (250 MHz,CDCl₃), δ in ppm: 1.48, 1.50 (d,3H, —CH₃); 2.42 ( br,1H, —OH); 5.30-5.37 (q,1H, —CH(CH₃)—); 7.30-7.83 (m,4H,Ar—H)

### 2) 1-(o-nitrophenyl)ethyl methyl thiomethyl ether (2-3) (C₁₀H₁₃NO₃S, Mw 227.28)

A solution of 1-(o-nitrophenyl)ethanol (5.52 g, 33.00 mmole) and chloromethyl methyl sulfide (3.82 g, 39.55 mmole) in 12 ml of dry benzene was added dropwise within 5 minutes to a solution of silver nitrate (6.17 g, 36.3 mmole) and triethyl amine (4.01 g, 39.63 mmole) in 20 ml of dry benzene. The solution was heated at 60° C. for 24 hours and subsequently filtered through a dry Celite column. The solution was extracted with dichloromethane and washed with 3% aqueous phosphoric acid, saturated aqueous monosodium carbonate and water and subsequently dried. The residue was purified by column chromatography on silica gel (dichloromethane: n-hexane=1:2).

Product: 1.70 g (22.67%)

TLC ( MC:Hex=3:1): Rf=0.5. ¹H-NMR (400 MHz,CDCl₃), δ in ppm: 1.50, 1.52 (d,3H,Ar—CH(CH₃)—); 2.10 (s,3H, —S—CH₃); 4.29,4.31,4.59,4.62 (dd,2H, —O—CH₂—S); 5.37,5.39,5.40,5.42 (q,1H, —CH(CH₃)—O); 7.39-7.90 (m,4H,Ar—H)

### 3) 1-(o-nitrophenyl)ethyl chloromethyl ether (2-4) (C₉H₁₀NO₃Cl, Mw 215.64)

Freshly distilled sulfur chloride (1.57 g, 11.35 mmole) in 10 ml dry dichloromethane was added dropwise at room temperature within 10 minutes to a solution of pure 1-(o-nitrophenyl)ethyl methyl thiomethyl ether in 15 ml of dichloromethane and subsequently stirred for one hour. The solution was evaporated by means of a rotary evaporator. The residue was distilled by means of a bulb tube oven at 100-110° C. and 0.05 torr. The product cannot be stored for a long period of time without distillation.

Product: 2.09 g (91%)

TLC (M ): Rf=0.07. ¹H-NMR (250 MHz,CDCl₃), δ in ppm: 1.52,1.54 (d,3H, —CH(CH₃)—O—); 5.17,5.18 (d,1H—O—CH₂—Cl); 5.36-5.52 (m,3H, —CH(CH₃)—O—CH₂—), 7.34-7.91 (m,4H,Ar—H)

### 4) 1-{5′-O-(4,4′-dimethoxytrityl)-2′-O-{[(1-(o-nitrobenzyl)ethyl)oxy]methyl}-β-L-ribofuranosyl}uracil(2-5) and 1-{5′-O-(4,4′-dimethoxytrityl)-3′-O-{[(1-(o-nitrobenzyl)ethyl)oxy]methyl}-β-L-ribofuranosyl}uracil (2-5-1) (C₃₉H₃₉N₃O₁₁, Mw 725.75)

Ethyl diisopropyl amine (1.55 g, 12 mmole) and Bu₂SnCl₂ (0.875 g, 2.88 mmole) were added to substance A in 12 ml of 1,2-dichloromethane. The solution was stirred at room temperature for 90 minutes under argon and subsequently heated at 70° C.

1-(o-nitrophenyl)ethyl chloromethyl ether was added to the solution at 70° C. After 30 minutes, the mixture was extracted with dichloromethane, washed with saturated aqueous monosodium carbonate and dried. The residue was purified by column chromatography on silica gel (ethyl acetate: n-hexane=3:2).

Product: substance-5 740 mg (42.5%), substance-5-1 550 mg (31.6%)

TLC (Hex:EtOAc=1:9 ) Rf=0.68 (substance 5), 0.43 (substance 5-1).

¹H-NMR (2-5,400 MHz,CDCl₃), 6 in ppm: 1.55-1.57 (t,3H,—CH(CH₃); 2.62 (br.d,OH—C(3′); 3.55 (dd,H—C(5′); 3.81 (s,2MeO); 4.12 (br.d,OH—C(3′)); 4.11 (dd,H—C(2′)); 4.55 (br.q,H—C(3′)); 4.94,5.01 (2d,OCH₂O); 5.29 (d,H—C(5)); 6.02 (d,H—C(1′)); 6.82-6.88 (m,Ar-4H); 7.25-7.78 (m,Ar-12H); 7.96 (d,H—C(6)); 7.91-7.97 (m,Ar-1H). MS(MALDI)=747 [M+Na]⁺

### 5) 1-{5′-O-(4,4′-dimethoxytrityl)-2′-O-{[(1-(O-nitrobenzyl)ethyl)oxy]methyl}-β-L-ribofuranosyl}uracil 3′-[(2-cyanoethyl)-diisopropylphosphoramidite] (2-6) (C₄₈H₅₆N₅O₁₂P, Mw 925.97)

Ethyl diisopropyl amine (178 mg, 1.38 mmole) and diisopropyl phosphoramidite-B (78.4 mg, 0.331 mmole) were added to substance (2-5) in 3 ml of a mixture of 1.5 ml of dichloromethane and 1.5 ml of acetonitrile. The solution was stirred for 1 hour under argon, quenched with butanol, extracted with ethyl acetate, washed with 5% monosodium carbonate and saturated aqueous sodium chloride, dried and evaporated. The residue was dissolved in 3 ml of a mixture of 1.5 ml of dichloromethane and 1.5 ml of ethane, then added dropwise to 150 ml of cold pentane (ice bath) and is then filtered.

Product: 177 mg (92%)

TLC (Hex:EtOAc=2:3) Rf=0.35, 0.27.

¹H-NMR(400 Hz,CDCl₃), 67 in ppm: 1.01-1.19 (m,12H,Me₂CH); 1.52-1.57 (m,3H, —CH(CH₃)—O—); 2.40,2.47 (2t,2H,OCH₂CH₂CN); 3.38-3.67 (m,5H,Me₂CH, H—C(5′),OCH₂CH₂CN); 3.80,3.81 (2s,6H,MeO); 3.91 (m,1H,OCH₂CH₂CN) ; 4.11-4.24 (m,1H,H—C(4′)); 4.39-4.68 (m,3H,H—C(2′) and (3′)); 4.85-4.97 (m,4H,OCH₂O,ArCH₂); 5.14,5.20 (2d,1H,H—C(5)); 6.06 (d,0.5H,H—C(1′)); 6.16 (d,0.5H,H—C(1′)); 6.83-6.87 (m,Ar-4H); 7.22-7.45 (m,Ar-10H); 7.60,7.78 (2m,Ar-2H); 7.87,7.95 (2d,1H,H—C(6)); 7.98 (m,Ar-1H);

³¹P-NMR(162 MHz): 151.2, 150.5 MS(MALDI)=948 [M+Na]⁺

1. A probe array for qualitative and/or quantitative detection of target molecules in a sample by molecular interactions between probe molecules and target molecules on the probe array, comprising an array surface and probe molecules immobilised on the array surface at defined sites,
wherein the probe molecules have at least one label and at least one selectively cleavable bond between the site of their immobilisation on the array surface and the label.

2. The probe array of claim 1, wherein the probe molecules are selected from the group consisting of oligonucleotides, peptides, proteins and their analogues.

3. The probe array of claim 1, wherein the probe molecules are oligonucleotides.

4. The probe array of claim 3, wherein the oligonucleotides have a length of from 10 to 100 bases.

5. The probe array of claim 1, wherein the selectively cleavable bond is located approximately in the centre between the site of the immobilisation of the probe molecule on the array surface and the label.

6. The probe array of claim 1, wherein the selectively cleavable bond cannot be selectively cleaved by enzymatic methods.

7. The probe array of claim 1, wherein the selectively cleavable bond can be selectively cleaved by chemical and/or physical methods.

8. The probe array of claim 1, wherein the selectively cleavable bond can be selectively cleaved by the addition of acid anions, base cations, fluoride and/or heavy metal ions.

9. The probe array of claim 8, wherein the heavy metal ions comprise mercury ions and/or silver ions.

10. The probe array of claim 1, wherein the selectively cleavable bond can be selectively cleaved by photolysis.

11. The probe array of claim 1, wherein the probe molecules comprise a nucleic acid of the formula A₁-S-A₂, wherein S is a nucleic acid that comprises the at least one selectively cleavable bond, and A₁ and A₂ are any nucleic acids or nucleic acid analogues.

12. The probe array of claim 11, wherein S is a nucleotide dimer that is bridged by the selectively cleavable bond.

13. The probe array of claim 12, wherein S is selected from the group consisting of the following dimers having the formulae I and II: 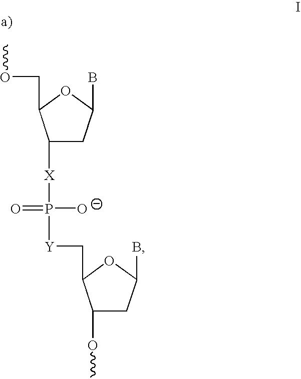
wherein X and Y are independently selected from the group consisting of O, NH and S, provided that X and Y are not simultaneously O; and B represents a nucleobase which is adenine, guanine, cytosine or thymine, 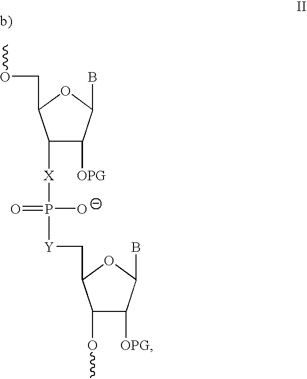
wherein X and Y are independently selected from the group consisting of O, NH and S, provided that X and Y are not simultaneously O, if PG is not a labile protective group; B represents a nucleobase which is adenine, guanine, cytosine or uracil; and PG is selected from the group consisting of H and labile protective groups such as or

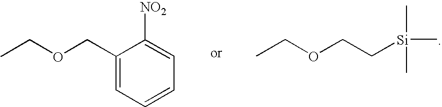

14. The probe array of claim 1, wherein the selectively cleavable bond is a phosphothioate bond.

15. The probe array of claim 1, wherein the label is a detectable unit, which is selected from the group consisting of fluorescent labels, luminescent labels, metal labels, enzyme labels, radioactive labels, polymeric labels and nucleic acids, which are detectable by hybridisation with a labelled reporter probe.

16. The probe array of claim 15, wherein the detectable unit is coupled to the probe molecules via an anchor group.

17. The probe array of claim 1, wherein the probe molecules are first probe molecules, and wherein said array further comprises second probe molecules arranged on at least one array element of the probe array, wherein the second probe molecules have at least one label and no selectively cleavable bond.

18. The probe array of claim 17, wherein the second probe molecules are oligonucleotides having a defined or randomised sequence.

19. The probe array of claim 1, further comprising an array element having arranged thereon detectable units that are not linked to a probe molecule.

20. The probe array of claim 17, wherein the second probe molecules are arranged on different array elements which differ in their labelling degree.

21. The probe array of claim 19, wherein the detectable units are arranged on different array elements which differ in their labelling degree.

22. The probe array of claim 1, further comprising third probe molecules which have no affinity or at least no specific affinity to target molecules arranged on at least one array element.

23. The probe array of claim 22, wherein the third probe molecules are oligonucleotides with a defined or randomised sequence.

24. The probe array of claim 1, further comprising fourth probe molecules arranged on at least one array element, and which have a specific affinity to spiking target molecules which are externally added to the sample.

25. The probe array of claim 24, comprising array elements distributed over the entire surface of the array, on which said fourth probe molecules are arranged, which have a label and a selectively cleavable bond located between the label and the immobilisation site of the probe on the surface and which have a specific affinity to spiking target molecule added externally to the sample or to a target molecule present in the sample in sufficient concentration.

26. A method for producing a probe array, comprising:
a) synthesizing probe molecules having a label and having a selectively cleavable bond between the site of their immobilisation on the array surface and the label; and
b) immobilizing the probe molecules via a defined position within the probe molecules at specific sites on the array surface.

27. A method for producing an array of probes on an array surface by in situ synthesis of the probe molecules on predetermined positions of the array surface, comprising:
a) providing an array surface which can be activated by suitable reagents or is provided with protective groups;
b) coupling or immobilising subunits of the probe molecules to be synthesised to predetermined sites on the array surface; and
c) synthesizing the probe molecules in situ following said coupling or immobilizing by incorporation of a label and a selectively cleavable bond between the site of the immobilisation of the probe molecules on the array surface and the label.

28. The method of claim 27, wherein said coupling is conducted by deposition of the subunit on the array surface.

29. The method of claim 27, wherein said coupling is preceded by activation or deprotection of the array surface.

30. The method of claim 27, the probes are covalently immobilized on the array surface.

31. The method of claim 27, wherein the probes comprise oligonucleotides.

32. The method of claim 31, wherein the oligonucleotide probes are synthesized according to the phosphoramidite method.

33. The method of claim 31, wherein the selectively cleavable bond is generated by bridging two nucleosides of the probe with a phosphothioate group.

34. The method of claim 27, wherein said synthesizing the probe molecules further comprises preparing a graduated labelling degree on an array element by adding a mixture of labelled monomers and unlabelled monomers, each of the same reactivity.

35. The method of claim 34, wherein the labelled monomers and the unlabelled monomers are mixed together in a defined ratio.

36. A method of controlling the quality of a probe array for qualitative and/or quantitative detection of target molecules in a sample by molecular interactions between probe molecules and target molecules on the probe array, comprising:
a) providing the probe array of claim 1; and
b) detecting the probe molecules in the form of signal intensities.

37. The method of claim 36, wherein said detecting is carried out via labels to be detected directly.

38. The method of claim 37, wherein said labels are fluorescent labels or radioactive labels.

39. The method of claim 36, further comprising determining an occupation density of the array elements with probe molecules by detecting the intensity of the signals generated by the labels.

40. The method of claim 36, wherein the detection takes place by way of imaging the signal intensities in the form of degrees of greyness.

41. The method of claim 36, further comprising storing results of the quality control in a database.

42. A method for qualitative and/or quantitative detection of target molecules from a sample to be analysed by molecular interactions between probe molecules and target molecules on probe arrays, comprising:
a) providing the probe array of claim 1;
b) optionally, detecting the probe molecules synthesised or immobilised on the array surface in the form of signal intensities;
c) incubating the probe array with the sample to be analysed;
d) optionally, washing under conditions, under which a specific interaction between the target molecules and the probe molecules remains largely stable and unspecifically bound targets are removed;
e) optionally, detecting the probe molecules in the form of signal intensities;
f) selectively cleaving the selectively cleavable bond in the probe molecules;
g) optionally, washing in order to remove labelled probe molecule fragments which are not retained by an interaction with target molecules on the array surface;
h) detecting the labelled probe molecule fragments which are retained on the array surface by an interaction with target molecules, in the form of signal intensities; and
i) optionally, standardizing the signal intensities obtained in h).

43. The method of claim 42, wherein the standardizing in i) is carried out by at least one of the following methods:
a) standardisation by mathematical combination of the signal intensities obtained in h) with a correction factor which is determined by the signal intensities obtained in b);
b) standardisation of mathematical combination of the intensities obtained in h) with a correction factor which is determined by the signal intensities of control array elements which are distributed over an entire area of the array and on which probe molecules are arranged, the probe molecules having a label and a selectively cleavable bond located between the labelling and the immobilisation site of the probe molecule on the array surface, wherein the probe molecules have a specific affinity to spiking target molecules externally added to the sample or a target molecule present in the sample in a sufficient concentration;
c) standardisation by subtraction of the signal intensities obtained in h) with the signal intensities detected of background array elements, on which probe molecules are arranged which undergo no or no detectable interaction with target molecules from the sample; and
d) standardisation by comparing the signal intensities obtained for an array element with the signal intensities of detection standard array elements, on which probe molecules are arranged, which are labelled, but not provided with a selectively cleavable bond.

44. The method of claim 42, wherein the degree of labelling of the detection standard array elements differ from the array elements in a characteristic manner.

45. The method of claim 42, wherein the selectively cleavable bond is selectively cleaved by chemical and/or physical methods.

46. The method of claim 45, wherein the selectively cleavable bond is selectively cleaved by the addition of acid anions, base cations, fluoride and/or heavy metal ions.

47. The method of claim 46, wherein the selectively cleavable bond is selectively cleaved by mercury and/or silver ions.

48. The method of claim 42, wherein the target molecules are fragmented by an enzymatic, physical or chemical method before said incubating.

49. The method of claim 42, wherein said incubating is carried out with a sample of labelled targets.

50. The method of claim 42, wherein said cleaving of the selectively cleavable bond is carried out at high ionic strength and/or low temperature.

51. A method for qualitative and/or quantitative detection of target molecules from a sample to be analysed by molecular interactions between probe molecules and target molecules on probe arrays, comprising:
a) providing the probe array of claim 1;
b) incubating the probe array with the sample to be analysed;
c) selectively cleaving the selectively cleavable bond in the probe molecules; and
d) detecting the labelled probe molecule fragments which are retained on the array surface by an interaction with target molecules, in the form of signal intensities.

52. A kit for qualitative and/or quantitative detection of target molecules from a sample by molecular interactions between probe molecules and target molecules on probe arrays, comprising:
a) the probe array of claim 1;
b) reagents for the selective cleavage of the selectively cleavable bond in the probe molecules;
c) hybridisation buffer; and
d) optionally, washing buffer.

53. The kit of claim 52, wherein the reagents are selected from the group consisting of heavy metal ions and enzymes.

54. The kit of claim 53, wherein the heavy metal ions are selected from mercury ions and/or silver ions.

55. The kit of claim 52, further comprising a reaction chamber.

56. The kit of claim 52, further comprising a detection device.

57. The kit of claim 52, further comprising a temperature control unit.

58. The kit of claim 52, wherein the probe array is in the form of a highly integrated autonomous unit.

59. A method for the production of monomer building blocks suitable for DNA synthesis, which can be used for the formation of a labile bond in probe molecules, comprising:
a) esterifying the 5′-OH group of a nucleoside with an acid suitable as leaving group, to produce an ester;
b) reacting the ester with a thioester;
c) saponifying the thioester to form a thiol;
d) protecting the thiol function with protective groups suitable for the phosphotriester or phosphoramidite method; and
e) activating the protected thiol at the 3′ position using the phosphotriester or phosphoramidite method.

60. A method for the production of monomer building blocks suitable for DNA synthesis, which can be used for the formation of a labile bond in probe molecules, comprising:
a) reacting a compound suitable as a protective group for the phosphotriester or phosphoramidite method to form a thiol;
b) esterifying the 5′-OH group of a nucleoside with an acid suitable as leaving group, to form an ester;
c) reacting the thiol of a) with the ester of b); and
d) activating the protected thiol at the 3′ position using the phosphotriester or phosphoramidite method.

61. The compound 5′-S-(dimethoxytrityl)-mercapto-5′-deoxynucleoside-3′-O-(2-cyanoethyl, N,N′-diisopropyl-phosphite.


SEQUENCE LISTING (70 sequences)

SEQ ID NO: 1 - DNA, 24 bp, Artificial Sequence
  Description of Artificial Sequence Synthetic oligonucleotide
agcccttactttgacggtatatct

SEQ ID NO: 2 - DNA, 10 bp, Artificial Sequence
  Description of Artificial Sequence Synthetic oligonucleotide
agcccttact

SEQ ID NO: 3 - DNA, 14 bp, Artificial Sequence
  Description of Artificial Sequence Synthetic oligonucleotide
ttgacggtatatct

SEQ ID NO: 4 - DNA, 24 bp, Artificial Sequence
  Description of Artificial Sequence Synthetic oligonucleotide
agcccttactttgacggtatatct

SEQ ID NO: 5 - DNA, 20 bp, Artificial Sequence
  Description of Artificial Sequence Synthetic oligonucleotide
gcagctagatataccgtcaa

SEQ ID NO: 6 - DNA, 20 bp, Artificial Sequence
  Description of Artificial Sequence Synthetic oligonucleotide
gctagatataccgtcaaagt

SEQ ID NO: 7 - DNA, 20 bp, Artificial Sequence
  Description of Artificial Sequence Synthetic oligonucleotide
gatataccgtcaaagtaagg

SEQ ID NO: 8 - DNA, 25 bp, Artificial Sequence
  Description of Artificial Sequence Synthetic probe
tctaaaacctggccagcaatcattc

SEQ ID NO: 9 - DNA, 25 bp, Artificial Sequence
  Description of Artificial Sequence Synthetic probe
gcccgggcatttctctcattaacat

SEQ ID NO: 10 - DNA, 25 bp, Artificial Sequence
  Description of Artificial Sequence Synthetic probe
ttcgaaaagattgcctccacatcag

SEQ ID NO: 11 - DNA, 25 bp, Artificial Sequence
  Description of Artificial Sequence Synthetic probe
gtctcatctttcttcacggagctgc

SEQ ID NO: 12 - DNA, 25 bp, Artificial Sequence
  Description of Artificial Sequence Synthetic probe
tgcttgtttgctctgttccttttca

SEQ ID NO: 13 - DNA, 25 bp, Artificial Sequence
  Description of Artificial Sequence Synthetic probe
tccaggttttccaggagagaatcca

SEQ ID NO: 14 - DNA, 25 bp, Artificial Sequence
  Description of Artificial Sequence Synthetic probe
tctgggtcagctccttcttaatggc

SEQ ID NO: 15 - DNA, 25 bp, Artificial Sequence
  Description of Artificial Sequence Synthetic probe
tctagaggatgcatttgacatgcca

SEQ ID NO: 16 - DNA, 25 bp, Artificial Sequence
  Description of Artificial Sequence Synthetic probe
tgttacatttgtgttgaactgcccc

SEQ ID NO: 17 - DNA, 25 bp, Artificial Sequence
  Description of Artificial Sequence Synthetic probe
aatgagattgcctttgcagttaggg

SEQ ID NO: 18 - DNA, 25 bp, Artificial Sequence
  Description of Artificial Sequence Synthetic probe
ttcttttgccctagctccaagttca

SEQ ID NO: 19 - DNA, 25 bp, Artificial Sequence
  Description of Artificial Sequence Synthetic probe
tcgtccaacaaatactttgcgatca

SEQ ID NO: 20 - DNA, 25 bp, Artificial Sequence
  Description of Artificial Sequence Synthetic probe
aatagctctttcagctgcttcctgc

SEQ ID NO: 21 - DNA, 25 bp, Artificial Sequence
  Description of Artificial Sequence Synthetic probe
tacaaatccatagcccttggaacca

SEQ ID NO: 22 - DNA, 25 bp, Artificial Sequence
  Description of Artificial Sequence Synthetic probe
tatgttgcctactccacttttgcga

SEQ ID NO: 23 - DNA, 25 bp, Artificial Sequence
  Description of Artificial Sequence Synthetic probe
tgttcaaatttgcgcttaagttccg

SEQ ID NO: 24 - DNA, 25 bp, Artificial Sequence
  Description of Artificial Sequence Synthetic probe
tttgttttccattgagctcctttcc

SEQ ID NO: 25 - DNA, 25 bp, Artificial Sequence
  Description of Artificial Sequence Synthetic probe
ttactttcacacttaaggcaggccc

SEQ ID NO: 26 - DNA, 25 bp, Artificial Sequence
  Description of Artificial Sequence Synthetic probe
gacatgactcgtggaacctgtgaag

SEQ ID NO: 27 - DNA, 25 bp, Artificial Sequence
  Description of Artificial Sequence Synthetic probe
taaatggtggtctaggagcagctgg

SEQ ID NO: 28 - DNA, 25 bp, Artificial Sequence
  Description of Artificial Sequence Synthetic probe
ttggctaggaggatagtatgcagca

SEQ ID NO: 29 - DNA, 25 bp, Artificial Sequence
  Description of Artificial Sequence Synthetic probe
aacacagcgtgttgctaacacatca

SEQ ID NO: 30 - DNA, 25 bp, Artificial Sequence
  Description of Artificial Sequence Synthetic probe
ctgtccgcaccgttccacagtataa

SEQ ID NO: 31 - DNA, 25 bp, Artificial Sequence
  Description of Artificial Sequence Synthetic probe
cagcaacatcttaatgcacagccac

SEQ ID NO: 32 - DNA, 25 bp, Artificial Sequence
  Description of Artificial Sequence Synthetic probe
aagttacaatgcaacagcctgctgt

SEQ ID NO: 33 - DNA, 30 bp, Artificial Sequence
  Description of Artificial Sequence Synthetic probe
tctaaaacctggccagcaatcattctgcca

SEQ ID NO: 34 - DNA, 30 bp, Artificial Sequence
  Description of Artificial Sequence Synthetic probe
ctctcctgctacagcagcccgggcatttct

SEQ ID NO: 35 - DNA, 30 bp, Artificial Sequence
  Description of Artificial Sequence Synthetic probe
cgaaggcaaagcccttatgaacagagcagc

SEQ ID NO: 36 - DNA, 30 bp, Artificial Sequence
  Description of Artificial Sequence Synthetic probe
tcccaatgaatacacgggagttcatggagc

SEQ ID NO: 37 - DNA, 30 bp, Artificial Sequence
  Description of Artificial Sequence Synthetic probe
ggatctgtcttgttggtaacgttgctggcc

SEQ ID NO: 38 - DNA, 30 bp, Artificial Sequence
  Description of Artificial Sequence Synthetic probe
tcatctttcttcacggagctgctgctctgc

SEQ ID NO: 39 - DNA, 30 bp, Artificial Sequence
  Description of Artificial Sequence Synthetic probe
tgggtcagctccttcttaatggcctgaagg

SEQ ID NO: 40 - DNA, 30 bp, Artificial Sequence
  Description of Artificial Sequence Synthetic probe
agaattgaagccacttttgccccttcgtga

SEQ ID NO: 41 - DNA, 30 bp, Artificial Sequence
  Description of Artificial Sequence Synthetic probe
tacatttgtgttgaactgccccacacagca

SEQ ID NO: 42 - DNA, 30 bp, Artificial Sequence
  Description of Artificial Sequence Synthetic probe
tcaaaggaagtgaaaatgggactaggcgcg

SEQ ID NO: 43 - DNA, 30 bp, Artificial Sequence
  Description of Artificial Sequence Synthetic probe
atgtgcttaagagtcatcctcgccattggc

SEQ ID NO: 44 - DNA, 30 bp, Artificial Sequence
  Description of Artificial Sequence Synthetic probe
agctctttcagctgcttcctgcgtctcaaa

SEQ ID NO: 45 - DNA, 30 bp, Artificial Sequence
  Description of Artificial Sequence Synthetic probe
actccacttttgcgaagtgatggatcacgc

SEQ ID NO: 46 - DNA, 30 bp, Artificial Sequence
  Description of Artificial Sequence Synthetic probe
gagaccacatgatgcgtactggcttgccct

SEQ ID NO: 47 - DNA, 30 bp, Artificial Sequence
  Description of Artificial Sequence Synthetic probe
tcaaaattcatggtgtccaaagcacgctcc

SEQ ID NO: 48 - DNA, 30 bp, Artificial Sequence
  Description of Artificial Sequence Synthetic probe
gccggctgctggaagttcacatacgcgtag

SEQ ID NO: 49 - DNA, 30 bp, Artificial Sequence
  Description of Artificial Sequence Synthetic probe
ttcaaatttgcgcttaagttccgtctgccg

SEQ ID NO: 50 - DNA, 30 bp, Artificial Sequence
  Description of Artificial Sequence Synthetic probe
ttgagctcctttccgttcatctcatccaca

SEQ ID NO: 51 - DNA, 30 bp, Artificial Sequence
  Description of Artificial Sequence Synthetic probe
aaggcgctcatcatccatgtcttctccaaa

SEQ ID NO: 52 - DNA, 30 bp, Artificial Sequence
  Description of Artificial Sequence Synthetic probe
catgactcgtggaacctgtgaagaagctgg

SEQ ID NO: 53 - DNA, 30 bp, Artificial Sequence
  Description of Artificial Sequence Synthetic probe
actaaatggtggtctaggagcagctgggcg

SEQ ID NO: 54 - DNA, 30 bp, Artificial Sequence
  Description of Artificial Sequence Synthetic probe
agcaccgggcatattttggaatggatgagg

SEQ ID NO: 55 - DNA, 30 bp, Artificial Sequence
  Description of Artificial Sequence Synthetic probe
accctgagcagtccagcgaggacttggtct

SEQ ID NO: 56 - DNA, 30 bp, Artificial Sequence
  Description of Artificial Sequence Synthetic probe
ctactcctgctgtccgcaccgttccacagt

SEQ ID NO: 57 - DNA, 30 bp, Artificial Sequence
  Description of Artificial Sequence Synthetic probe
tgcaggagttcgcaatcctcagcaacatct

SEQ ID NO: 58 - DNA, 30 bp, Artificial Sequence
  Description of Artificial Sequence Synthetic probe
tgcacagccacaagttacaatgcaacagcc

SEQ ID NO: 59 - DNA, 30 bp, Artificial Sequence
  Description of Artificial Sequence Synthetic probe
tcaggaacctttgactgcttccatgttggc

SEQ ID NO: 60 - DNA, 20 bp, Artificial Sequence
  Description of Artificial Sequence Synthetic probe
cctctgcagactactattac

SEQ ID NO: 61 - RNA, 612 nt, Artificial Sequence
  Description of Artificial Sequence Synthetic hybridization target
ucuagaaauaauuuuguuuaacuuuaagaaggagauauacauaugaaccccagugcccccagcuaccccauggccucgcucuacgugggggaccuccaccccgacgugaccgaggcgaugcucuacgagaaguucagcccggccgggcccauccucuccauccgggucugcagggacaugaucacccgccgcuccuugggcuacgcguaugugaacuuccagcagccggcggacgcggagcgugcuuuggacaccaugaauuuugauguuauaaagggcaagccaguacgcaucauguggucucagcgugauccaucacuucgcaaaaguggaguaggcaacauauucauuaaaaaucuggacaaauccauugauaauaaagcacuguaugauacauuuucugcuuuugguaacauccuuucauguaaggugguuugugaugaaaaugguuccaagggcuauggauuuguacacuuugagacgcaggaagcagcugaaagagcuauugaaaaaaugaauggaaugcuccuaaaugaucgcaaaguauuuguuggacgauuuaagucucguaaagaacgagaagcugaacuuggagcuagggcaaaagaauuc

SEQ ID NO: 62 - RNA, 449 nt, Artificial Sequence
  Description of Artificial Sequence Synthetic hybridization target
aacugcuuucugggcagccucuuuagcuuggugggcuuguaguacagcuacagcuucaucaaccuuagaacggagugacucuggagacucgagcauaugaagaaguucugaauuaucaaucuccaacaacaugccagugauuuuaccagcaagaguagggugcauggcuugaauaagaggaaacagccguucacccaacauuugcuuuugcucuugaggaggggcagaugccaacauggaagcagucaaagguuccugaccuuguacaugaacagcaggcuguugcauuguaacuuguggcugugcauuaagauguugcugaggauugcgaacuccugcagcauauuuauacuguggaacggugcggacagcaggaguagcugcagcggcugcagcugcaggacguggacccauugucuguguugauguguuagcaacacgcuguguug

SEQ ID NO: 63 - RNA, 156 nt, Artificial Sequence
  Description of Artificial Sequence Synthetic hybridization target
ucuagaaaaauaauuaguguuauagucuuaagauuuguuuucuaaaguugauacuguggguuauuuuugugaacagccugauguuugggaccuuuuuuccucaaaauaaacaaguccuuauuaaaccaggaauuuggagaaaaaaaaaaggaauuc

SEQ ID NO: 64 - RNA, 380 nt, Artificial Sequence
  Description of Artificial Sequence Synthetic hybridization target
gaauuccaaacccgggaguaggagacucagaaucgaaucucuucucccuccccuucuugugagauuuuuuugaucuucagcuacauuuucggcuuugugagaaaccuuaccaucaaacacgauggccagcaacguuaccaacaagacagauccucgcuccaugaacucccguguauucauugggaaucucaacacucuuguggucaagaaaucugauguggaggcaaucuuuucgaaguauggcaaaauugugggcugcucuguucauaagggcuuugccuucguucaguauguuaaugagagaaaugcccgggcugcuguagcaggagaggauggcagaaugauugcuggccagguuuuagauauuaaccuggcugcag

SEQ ID NO: 65 - RNA, 401 nt, Artificial Sequence
  Description of Artificial Sequence Synthetic hybridization target
gaauucaccaauguuuacaucaagaauuuuggagaagacauggaugaugagcgccuuaaggaucucuuuggcaaguuugggccugccuuaagugugaaaguaaugacugaugaaaguggaaaauccaaaggauuuggauuuguaagcuuugaaaggcaugaagaugcacagaaagcuguggaugagaugaacggaaaggagcucaauggaaaacaaauuuauguuggucgagcucagaaaaagguggaacggcagacggaacuuaagcgcaaauuugaacagaugaaacaagauaggaucaccagauaccaggguguuaaucuuuaugugaaaaaucuugaugaugguauugaugaugaacgucuccggaaagaguuuucuccauuugguacaaucacuag

SEQ ID NO: 66 - RNA, 470 nt, Artificial Sequence
  Description of Artificial Sequence Synthetic hybridization target
agugcaaagguuaugauggaggguggucgcagcaaaggguuugguuuuguauguuucuccuccccagaagaagccacuaaagcaguuacagaaaugaacgguagaauuguggccacaaagccauuguauguagcuuuagcucagcgcaaagaagagcgccaggcucaccucacuaaccaguauaugcagagaauggcaaguguacgagcuguucccaacccuguaaucaaccccuaccagccagcaccuccuucagguuacuucauggcagcuaucccacagacucagaaccgugcugcauacuauccuccuagccaaauugcucaacuaagaccaaguccucgcuggacugcucagggugccagaccucauccauuccaaaauaugcccggugcuauccgcccagcugcuccuagaccaccauuuaguacuaugagaccagcuucuucacagguuccacgagucauguc

SEQ ID NO: 67 - RNA, 503 nt, Artificial Sequence
  Description of Artificial Sequence Synthetic hybridization target
cugcagcggagauguacggcuccucuuuugacuuggacuaugacuuucaacgggacuauuaugauaggauguacaguuacccagcacguguaccuccuccuccuccuauugcucgggcuguagugcccucgaaacgucagcguguaucaggaaacacuucacgaaggggcaaaaguggcuucaauucuaagaguggacagcggggaucuuccaagucuggaaaguugaaaggagaugaccuucaggccauuaagaaggagcugacccagauaaaacaaaaaguggauucucuccuggaaaaccuggaaaaaauugaaaaggaacagagcaaacaagcaguagagaugaagaaugauaagucagaagaggagcagagcagcagcuccgugaagaaagaugagacuaaugugaagauggagucugaggggggugcagaugacucugcugaggagggggaccuacuggaugaugaugauaaugaagaucggggggaugaccagcug

SEQ ID NO: 68 - RNA, 620 nt, Artificial Sequence
  Description of Artificial Sequence Synthetic hybridization target
cagcuggaguugaucaaggaugaugaaaaagaggcugaggaaggagaggaugacagagacagcgccaauggcgaggaugacucuuaagcacauagugggguuuagaaaucuuaucccauuauuucuuuaccuaggcgcuugucuaagaucaaauuuuucaccagauccucuccccuaguaucuucagcacaugcucacuguucuccccauccuuguccuucccauguucauuaauucauauugccccgcgccuagucccauuuucacuuccuuugacgcuccuaguaguuuuguuaagucuuacccugaaauuuuugcuuuuaauuuugauaccucuuuaugacuuaacaauaaaaaggauguaugguuuuuaucaacugucuccaaaauaaucucuuguuaugcagggaguacaguucuuuucauucauacauaaguucaguaguugcuucccuaacugcaaaggcaaucucauuuaguugaguagcucuugaaagcagcuuugaguuagaaguauguguguuacacccucacauuagugugcuguguggggcaguucaacacaaauguaacaauuauuuuugugaaugagaguuggcaugucaaaugcauccucuaga

SEQ ID NO: 69 - DNA, 12 bp, Artificial Sequence
  Description of Artificial Sequence Synthetic oligonucleotide
gacggtatatct

SEQ ID NO: 70 - DNA, 9 bp, Artificial Sequence
  Description of Artificial Sequence Synthetic oligonucleotide
agcccttac